(19) United States
(12) Patent Application Publication
(10) Pub. No.: US 2009/0191228 A1
(43) Pub. Date: Jul. 30, 2009

(54) Immunogenic compositions capable of activating T-cells

(75) Inventors: Martijn Frans Ben Gerard Gebbink, Eemnes (NL); Barend Bouma, Houten (NL); Paulus Johannes Gerardus Maria Steverink, Huizen (NL); Johan Renes, Amersfoort (NL)

Correspondence Address: TRASKBRITT, P.C., P.O. BOX 2550, SALT LAKE CITY, UT, 84110, US

(73) Assignee: Crossbeta Biosciences B.V., Utrecht (NL)

(21) Appl. No.: 12/291,398

(22) Filed: Nov. 7, 2008

(30) Foreign Application Priority Data
Nov. 8, 2007 (EP) 07120289.9

(51) Int. Cl. A61K 39/00; A61K 38/02; A61K 38/14; A61K 38/16; G01N 33/68; G01N 33/92; A61P 37/00

(52) U.S. Cl. 424/184.1; 514/2; 514/8; 514/12; 436/86; 436/71; 436/87

(57) ABSTRACT

Provided is means and methods for producing and/or selecting immunogenic compositions capable of activating a T-cell and/or a T-cell response, comprising providing the composition with at least one cross-beta structure and testing at least one immunogenic property.

### CROSS-REFERENCE TO RELATED APPLICATION

This application claims priority to European Patent Application Serial No. EP 07120289.9, filed Nov. 8, 2007, the entire contents of which is hereby incorporated herein by this reference.

### TECHNICAL FIELD

The invention relates to the fields of cell biology, immunology, vaccinology, adjuvant technology and medicine.

### BACKGROUND

Vaccines can be divided in two basic groups, i.e., prophylactic vaccines and therapeutic vaccines. Prophylactic vaccines have been made and/or suggested against essentially every known infectious agent (virus, bacterium, yeast, fungi, parasite, mycoplasm, etc.), which has some pathology in man, pets and/or livestock, which infectious agent is therefore also referred to as “pathogen.” Therapeutic vaccines have been made and/or suggested for infectious agents as well, but also for treatments of cancer and other aberrancies, as well as for inducing immune responses against other self antigens, as widely ranging as, e.g., LHRH for immunocastration of boars, or for use in preventing graft versus host (GvH) and/or transplant rejections.

In vaccines in general there are two vital issues. Vaccines have to be efficacious and vaccines have to be safe. It often seems that the two requirements are mutually exclusive when trying to develop a vaccine. The most efficacious vaccines so far have been modified live infectious agents. These are modified in a manner that their virulence has been reduced (attenuation) to an acceptable level. The vaccine strain of the infectious agent typically does replicate in the host, but at a reduced level, so that the host can mount an adequate immune response, also providing the host with long term immunity against the infectious agent. The downside of attenuated vaccines is that the infectious agents may revert to a more virulent (and thus pathogenic) form.

This may happen in any infectious agent, but is a very serious problem in fast mutating viruses (such as in particular RNA viruses). Another problem with modified live vaccines is that infectious agents often have many different serotypes. It has proven to be difficult in many cases to provide vaccines which elicit an immune response in a host that protects against different serotypes of infectious agents.

Vaccines in which the infectious agent has been killed are often safe, but often their efficacy is mediocre at best, even when the vaccine contains an adjuvant. In general an immune response is enhanced by adding adjuvants (from the Latin adjuvare, meaning “to help”) to the vaccines. The chemical nature of adjuvants, their proposed mode of action and their reactions (side effect) are highly variable. Some of the side effects can be ascribed to an unintentional stimulation of different mechanisms of the immune system whereas others may reflect general adverse pharmacological reactions which are more or less expected. There are several types of adjuvants. Today the most common adjuvants for human use are aluminium hydroxide, aluminium phosphate and calcium phosphate. However, there is a number of other adjuvants based on oil emulsions, products from bacteria (their synthetic derivatives as well as liposomes) or gram-negative bacteria, endotoxins, cholesterol, fatty acids, aliphatic amines, paraffinic and vegetable oils. Recently, monophosphoryl lipid A, ISCOMs with Quil-A, and Syntex adjuvant formulations (SAFs) containing the threonyl derivative or muramyl dipeptide have been under consideration for use in human vaccines. Chemically, adjuvants are a highly heterogenous group of compounds with only one thing in common: their ability to enhance the immune response—their adjuvanticity. They are highly variable in terms of how they affect the immune system and how serious their adverse effects are due to the resultant hyperactivation of the immune system. The choice of any of these adjuvants reflects a compromise between a requirement for adjuvanticity and an acceptable low level of adverse reactions. The term “adjuvant” has been used for any material that is capable of increasing the humoral and/or cellular immune response to an antigen. In the conventional vaccines, adjuvants are used to elicit an early, high and long-lasting immune response. The newly developed purified subunit or synthetic vaccines (see below) using biosynthetic, recombinant and other modern technology are poor immunogens and require adjuvants to evoke the immune response. The use of adjuvants enables the use of less antigen to achieve the desired immune response, and this reduces vaccine production costs. With a few exceptions, adjuvants are foreign to the body and cause adverse reactions.

A type of vaccine that has received a lot of attention since the advent of modern biology is the subunit vaccine. In these vaccines only one or a few elements of the infectious agent are used to elicit an immune response. Typically a subunit vaccine comprises one, two or three proteins, glycoproteins and/or peptides present in proteins, or fragments thereof, of an infectious agent (from one or more serotypes) which have been purified from a pathogen or produced by recombinant means and/or synthetic means. Although these vaccines in theory are the most promising safe and efficacious vaccines, in practice efficacy has proved to be a major hurdle. Molecular biology has provided more alternative methods to arrive at safe and efficacious vaccines that theoretically should also provide cross-protection against different serotypes of infectious agents. Carbohydrate structures derived from infectious agents have been suggested as specific immune response eliciting components of vaccines, as well as lipopolysaccharide structures, and even nucleic acid complexes have been proposed. Although these component vaccines are generally safe, their efficacy and cross-protection over different serotypes has been generally lacking. Combinations of different kinds of components have been suggested (carbohydrates with peptides/proteins and lipopolysaccharide (LPS) with peptides/proteins optionally with carriers), but so far the safety vs. efficacy issue remains.

Another approach to provide cross-protection is to make hybrid infectious agents which comprise antigenic components from two or more serotypes of an infectious agent. These can be and have been produced by modern molecular biology techniques. They can be produced as modified live vaccines, or as vaccines with inactivated or killed pathogens, but also as subunit vaccines. Cocktail or combination vaccines comprising antigens from completely different infectious agents are also well known. In many countries children are routinely vaccinated with cocktail vaccines against e.g., diphtheria, whooping cough, tetanus and polio. Recombinant vaccines comprising antigenic elements from different infectious agents have also been suggested. For instance for poultry a vaccine based on a chicken anemia virus has been suggested to be complemented with antigenic elements of Marek disease virus (MDV), but many more combinations have been suggested and produced.

Another important advantage of modern recombinant vaccines is that they have provided the opportunity to produce marker vaccines. Marker vaccines have been provided with an extra element that is not present in wild type infectious agent, or marker vaccines lack an element that is present in wild type infectious agent. The response of a host to both types of marker vaccines can be distinguished (typically by serological diagnosis) from the response against an infection with wild type.

An efficient way of producing immunogenic compositions, or improving the immunogenicity of immunogenic compositions, has been provided in WO 2007/008070, the contents of the entirety of which are incorporated herein by this reference. This patent application discloses that the immunogenicity of a composition which comprises amino acid sequences is enhanced by providing the composition with at least one cross-beta structure. A cross-beta structure is a structural element of peptides and proteins, comprising stacked beta sheets, as will be discussed in more detail below. According to WO 2007/008070, the presence of cross-beta structure enhances the immunogenicity of a composition comprising an amino acid sequence. An immunogenic composition is thus prepared by producing a composition which comprises an amino acid sequence, such as a protein containing composition, and administrating (protein comprising) cross-beta structures to the composition. Additionally, or alternatively, cross-beta structure formation in the composition is induced, for instance by changing the pH, salt concentration, reducing agent concentration, temperature, buffer and/or chaotropic agent concentration, and/or combinations of these parameters.

The methods disclosed in WO 2007/008070 are suitable for the production of immunogenic compositions capable of eliciting and/or stimulating a humoral immune response, as well as for the production of immunogenic compositions capable of eliciting and/or stimulating a cellular immune response. A schematic overview of a humoral and a cellular immune response is given in FIG. 11. A humoral immune response, including the production of antigen-specific antibodies, is often elicited against extracellular pathogens such as virus, bacteria, yeast, fungi, parasite and mycoplasm whereas a cellular immune response, including the production of a cytotoxic T-cell response, is often elicited against intracellular pathogens, cancer and self-antigens.

### SUMMARY OF THE INVENTION

Provided are improved means and methods for producing and/or improving immunogenic compositions. Further provided are compositions with enhanced immunogenicity for use as vaccines.

In certain embodiments, provided are improved methods for providing and/or selecting immunogenic compositions which are capable of activating T-cells, for example resulting in a CD4+ T-help response, and/or resulting in a CD8+ cytotoxic T-lymphocyte response. T-cell epitopes are not always known. When T-cell epitope motifs are not known for a given protein, T-cell epitope motifs are preferably predicted. It is possible to predict T-cell epitopes for CD4+ related T-cell activation as well as for T-cell epitopes for CD8+ related T-cell activation. It is known in the art how T-cell epitopes capable of inducing an MHC-I mediated T-cell activation as well as T-cell epitopes capable of inducing an MHC-II mediated T-cell activation are predicted. This is for instance done by screening the primary sequence of a compound comprising an amino acid sequence such as, but not limited to, a peptide, polypeptide, protein, glycoprotein, protein-DNA complex, protein-membrane complex and/or lipoprotein, and in summary referred to as “protein,” for the presence of peptides with a length of between 5-30 amino-acid residues, preferably flanked by sequences which are capable of being recognized and cleaved by an MHC antigen processing pathway. It is preferably also determined whether putative T-cell epitopes have anchor residues so that the epitopes can be bound to a component of an MHC antigen processing pathway and be presented by an antigen presenting cell. Putative T-cell epitope motifs are for example obtained by synthesizing peptides covering overlapping sequences of the antigen, comprising preferably the number of amino-acid residues known to be required for presentation by major histocompatibility complexes, for example 5-30 amino-acid residues. The sequence overlap between two adjacent peptides is for example 1-10 amino-acid residues. Algorithms and computer based analysis techniques are often used in order to determine whether a protein comprises T-cell epitope motifs.

In certain aspects, at least one peptide, polypeptide, protein, glycoprotein, protein-DNA complex, protein-membrane complex and/or lipoprotein comprising at least one predicted and/or putative T-cell epitope motif is selected and incorporated into one or more compositions. Subsequently, the composition is provided with at least one cross-beta structure. This way, an immunogenic composition capable of eliciting and/or stimulating a cellular immune response is obtained.

One embodiment of the invention thus provides a method for producing an immunogenic composition comprising at least one peptide, polypeptide, protein, glycoprotein, protein-DNA complex, protein-membrane complex and/or lipoprotein, the method comprising: determining whether a peptide, polypeptide, protein, glycoprotein, protein-DNA complex, protein-membrane complex and/or lipoprotein comprises a T-cell epitope motif; selecting a peptide, polypeptide, protein, glycoprotein, protein-DNA complex, protein-membrane complex and/or lipoprotein comprising a T-cell epitope motif; providing a composition comprising the selected peptide, polypeptide, protein, glycoprotein, protein-DNA complex, protein-membrane complex and/or lipoprotein; and providing the composition with at least one cross-beta structure.

One advantage of the use of a cross-beta structure is that the use of adjuvants in order to induce an immune response is reduced or no longer necessary (although such adjuvant may still be used at will).

It is also possible to use a peptide, polypeptide, protein, glycoprotein, protein-DNA complex, protein-membrane complex and/or lipoprotein, which is known to comprise a T-cell epitope. A composition comprising such T-cell epitope comprising compound is provided with cross-beta structures in order to obtain an immunogenic compound.

In certain embodiments, the T-cell epitope comprises a cytotoxic T lymphocyte (CTL) epitope. This way an immunogenic composition capable of eliciting and/or stimulating a cellular immune response is obtained.

Alternatively, or additionally, the T-cell epitope comprises a T-helper cell epitope. In this embodiment an immunogenic composition capable of eliciting and/or stimulating a humoral immune response is obtained.

Also provided are improved methods for providing an immunogenic composition capable of activating T-cells and/or a T-cell response, the method comprising providing an amino acid containing composition with at least one cross-beta structure and subsequently testing at least one, preferably at least two, immunogenic properties of the resulting composition. Thus provided a ways for controlling a process for the production of an immunogenic composition, so that immunogenic compositions with preferred immunogenic properties are produced and/or selected. In this embodiment a peptide, polypeptide, protein, glycoprotein, protein-DNA complex, protein-membrane complex and/or lipoprotein comprising a known T-cell epitope and/or a predicted or determined T-cell epitope motif is used. Provided is a method wherein a composition comprising at least one amino acid sequence such as, but not limited to, a peptide, polypeptide, protein, glycoprotein, protein-DNA complex, protein-membrane complex and/or lipoprotein, which comprises a T-cell epitope and/or a T-cell epitope motif, is provided with at least one cross-beta structure, where after at least one of the following properties is tested: whether the degree of multimerization of the peptide, polypeptide, protein, glycoprotein, protein-DNA complex, protein-membrane complex and/or lipoprotein in the composition allows recognition, excision, processing and/or presentation of a T-cell epitope of the peptide, polypeptide, protein, glycoprotein, protein-DNA complex, protein-membrane complex and/or lipoprotein by an animal's immune system; whether between 4-75% of the peptide, polypeptide, protein, glycoprotein, protein-DNA complex, protein-membrane complex and/or lipoprotein content of the composition is in a conformation comprising cross-beta structures; whether the at least one cross-beta structure comprises a property allowing recognition, excision, processing and/or presentation of a T-cell epitope of the peptide, polypeptide, protein, glycoprotein and/or lipoprotein by an animal's immune system; and/or whether a compound capable of specifically binding, recognizing, excising, processing and/or presenting a T-cell epitope of the peptide, polypeptide, protein, glycoprotein, protein-DNA complex, protein-membrane complex and/or lipoprotein is capable of specifically binding, recognizing, excising, processing and/or presenting the immunogenic composition. This is outlined below in more detail.

Cross-beta structures are present in a subset of misfolded proteins such as for instance amyloid. A misfolded protein is defined herein as a protein with a structure other than a native, non-amyloid, non-cross-beta structure. Hence, a misfolded protein is a protein having a non-native three dimensional structure, and/or a cross-beta structure, and/or an amyloid structure.

Misfolded proteins tend to multimerize and can initiate fibrillization. This can result in the formation of amorphous aggregates that can vary greatly in size. In certain cases misfolded proteins are more regular and fibrillar in nature. The term “amyloid” has initially been introduced to define the fibrils, which are formed from misfolded proteins, and which are found in organs and tissues of patients with the various known misfolding diseases, collectively termed amyloidoses. Commonly, amyloid appears as fibrils with undefined length and with a mean diameter of 10 nm, is deposited extracellularly, stains with the dyes Congo red and Thioflavin T (ThT), shows characteristic green birefringence under polarized light when Congo red is bound, comprises beta-sheet secondary structure, and contains the characteristic cross-beta conformation (see below) as determined by X-ray fiber diffraction analysis. However, since it has been determined that protein misfolding is a more general phenomenon and since many characteristics of misfolded proteins are shared with amyloid, the term amyloid has been used in a broader scope. Now, the term “amyloid” is also used to define intracellular fibrils and fibrils formed in vitro. Also the terms “amyloid-like” and “amylog” are used to indicate misfolded proteins with properties shared with amyloids, but that do not fulfill all criteria for amyloid, as listed above.

In conclusion, misfolded proteins are highly heterogeneous in nature, ranging from monomeric misfolded proteins, to small oligomeric species, sometimes referred to as protofibrils, larger aggregates with amorphous appearance, up to large highly ordered fibrils, all of which appearances can share structural features reminiscent to amyloid. As used herein, the term “misfoldome” encompasses any collection of misfolded proteins.

Amyloid and misfolded proteins that do not fulfill all criteria for being identified as amyloid can share structural and functional features with amyloid and/or with other misfolded proteins. These common features are shared among various misfolded proteins, independent of their varying amino acid sequences. Shared structural features include for example the binding to certain dyes, such as Congo red, ThT, Thioflavin S, accompanied by enhanced fluorescence of the dyes, multimerization, and the binding to certain proteins, such as tissue-type plasminogen activator (tPA), the receptor for advanced glycation end-products (RAGE) and chaperones, such as heat shock proteins, like BiP (grp78 or immunoglobulin heavy chain binding protein). Shared functional activities include the activation of tPA and the induction of cellular responses, such as inflammatory responses and an immune response, and induction of cell toxicity.

A unique hallmark of a subset of misfolded proteins such as for instance amyloid is the presence of the cross-beta conformation or a precursor form of the cross-beta conformation.

A cross-beta structure is a secondary structural element in peptides and proteins. A cross-beta structure (also referred to as a “cross-β,” a “cross-beta” or a “cross-structure”) is defined as a part of a protein or peptide, or a part of an assembly of peptides and/or proteins, which comprises single beta-strands (stage 1) and a(n ordered) group of beta-strands (stage 2), and typically a group of beta-strands, preferably composed of 5-10 beta-strands, arranged in a beta-sheet (stage 3). A cross-beta structure often comprises in particular a group of stacked beta-sheets (stage 4), also referred to as “amyloid.” Typically, in cross-beta structures the stacked beta sheets comprise flat beta sheets in a sense that the screw axis present in beta sheets of native proteins, is partly or completely absent in the beta sheets of stacked beta sheets. A cross-beta structure is formed following formation of a cross-beta structure precursor form upon protein misfolding like for example denaturation, proteolysis or unfolding of proteins. A cross-beta structure precursor is defined as any protein conformation that precedes the formation of any of the aforementioned structural stages of a cross-beta structure. These structural elements present in cross-beta structure (precursor) are typically absent in globular regions of (native parts of) proteins. The presence of cross-beta structure is for example demonstrated with X-ray fibre diffraction or binding of ThT or binding of Congo red, accompanied by enhanced fluorescence of the dyes.

A typical form of a cross-beta structure precursor is a partially or completely misfolded protein. A typical form of a misfolded protein is a partially or completely unfolded protein, a partially refolded protein, a partially or completely aggregated protein, an oligomerized or multimerized protein, or a partially or completely denatured protein. A cross-beta structure or a cross-beta structure precursor can appear as monomeric molecules, dimeric, trimeric, up to oligomeric assemblies of molecules and can appear as multimeric structures and/or assemblies of molecules.

Cross-beta structure (precursor) in any of the aforementioned states can appear in soluble form in aqueous solutions and/or organic solvents and/or any other solutions. Cross-beta structure (precursor) can also be present as solid state material in solutions, like for example as insoluble aggregates, fibrils, particles, like for example as a suspension or separated in a solid cross-beta structure phase and a solvent phase.

Protein misfolding, formation of cross-beta structure precursor, formation of aggregates or multimers and/or cross-beta structure can occur in any composition comprising protein(s) and/or peptides with a length of at least 2 amino acids. The term “peptide” is intended to include oligopeptides as well as polypeptides, and the term “protein” includes proteinaceous molecules including peptides, with and without post-translational modifications such as for instance glycosylation, citrullination, oxidation, acetylation and glycation. It also includes lipoproteins and complexes comprising a proteinaceous part, such as for instance protein-nucleic acid complexes (RNA and/or DNA), membrane-protein complexes, etc. As used herein, the term “protein” also encompasses proteinaceous molecules, peptides, oligopeptides and polypeptides. Hence, the use of “protein” or “protein and/or peptide” in this application have the same meaning.

A typical form of stacked beta-sheets is in a fibril-like structure in which the beta-strands are oriented in either the direction of the fiber axis or perpendicular to the direction of the fiber axis. The direction of the stacking of the beta-sheets in cross-beta structures is perpendicular to the long fiber axis. A cross-beta structure conformation is a signal that triggers a cascade of events that induces clearance and breakdown of the obsolete protein or peptide. When clearance is inadequate, unwanted proteins and/or peptides aggregate and form toxic structures ranging from soluble oligomers up to precipitating fibrils and amorphous plaques. Such cross-beta structure conformation comprising aggregates underlie various diseases and disorders, such as for instance, Huntington's disease, amyloidosis type disease, atherosclerosis, cardiovascular disease, diabetes, bleeding, thrombosis, cancer, sepsis and other inflammatory diseases, rheumatoid arthritis, transmissible spongiform encephalopathies such as Creutzfeldt-Jakob disease, multiple sclerosis, auto-immune diseases, uveitis, ankylosing spondylitis, diseases associated with loss of memory such as Alzheimer's disease, Parkinson's disease and other neuronal diseases (epilepsy), encephalopathy and systemic amyloidoses.

A cross-beta structure is for instance formed during unfolding and refolding of proteins and peptides. Unfolding of peptides and proteins occur regularly within an organism. For instance, peptides and proteins often unfold and refold spontaneously at the end of their life cycle. Moreover, unfolding and/or refolding is induced by environmental factors such as for instance pH, glycation, oxidative stress, heat, irradiation, mechanical stress, proteolysis citrullination, ischeamia, and so on. As used herein, the terms “cross-beta” and “cross-beta structure” also encompasses any cross-beta structure precursor and any misfolded protein, even though a misfolded protein does not necessarily comprise a cross-beta structure. The term “cross-beta binding molecule” or “molecule capable of specifically binding a cross-beta structure” also encompasses a molecule capable of specifically binding any misfolded protein.

The terms “unfolding,” “refolding” and “misfolding” relate to the three-dimensional structure of a protein or peptide. Unfolding means that a protein or peptide loses at least part of its three-dimensional structure. The term refolding relates to the coiling back into some kind of three-dimensional structure. By refolding, a protein or peptide can regain its native configuration, or an incorrect refolding can occur. The term “incorrect refolding” refers to a situation when a three-dimensional structure other than a native configuration is formed. Incorrect refolding is also called misfolding. Unfolding and refolding of proteins and peptides involves the risk of cross-beta structure formation. Formation of cross-beta structures sometimes also occurs directly after protein synthesis, without a correctly folded protein intermediate.

In certain methods disclosed herein, an immunogenic composition comprising at least one peptide, polypeptide, protein, glycoprotein, protein-DNA complex, protein-membrane complex and/or lipoprotein is provided with at least one cross-beta structure. This is performed in various ways. For instance, a peptide, polypeptide, protein, glycoprotein, protein-DNA complex, protein-membrane complex and/or lipoprotein is subjected to a cross-beta inducing procedure, preferably a change of pH, salt concentration, reducing agent concentration, temperature, buffer and/or chaotropic agent concentration. These procedures are known to induce and/or enhance cross-beta formation. In certain embodiments, the peptide, polypeptide, protein, glycoprotein, protein-DNA complex, protein-membrane complex and/or lipoprotein is subjected to a cross-beta inducing procedure before it is used for the preparation of an immunogenic composition. It is, however, also possible to subject the peptide, polypeptide, protein, glycoprotein, protein-DNA complex, protein-membrane complex and/or lipoprotein to a cross-beta inducing procedure while it is already present in an immunogenic composition.

Additionally, or alternatively, a peptide, polypeptide, protein, glycoprotein, protein-DNA complex, protein-membrane complex and/or lipoprotein is provided with a (peptide or protein comprising a) cross-beta structure, either before it is used for the preparation of an immunogenic composition or after it has been used for the preparation of an immunogenic composition.

After an immunogenic composition according to the invention has been provided with cross-beta structures, one or more immunogenic properties of the resulting composition are tested.

In certain embodiments, tested is whether the degree of multimerization of the peptide, polypeptide, protein, glycoprotein, protein-DNA complex, protein-membrane complex and/or lipoprotein in the immunogenic composition allows recognition, excision, processing and/or presentation of a T-cell epitope of the peptide, polypeptide, protein, glycoprotein, protein-DNA complex, protein-membrane complex and/or lipoprotein by an animal's immune system. Proteins comprising cross-beta structures tend to multimerize. Hence, after an immunogenic composition has been provided with cross-beta structures, multimerization of the peptide, polypeptide, protein, glycoprotein, protein-DNA complex, protein-membrane complex and/or lipoprotein in the immunogenic composition will occur. In certain aspects, tested is whether the degree of multimerization is such that an animal's immune system is still capable of recognizing, excising, processing and/or presenting a T-cell epitope (of interest). For instance, too much multimerization will result in the formation of a fibril wherein T-cell epitopes are no longer accessible for protease systems, for example the MHC antigen processing pathway. Additionally, or alternatively, too much multimerization results in a decreased ability of the cross-beta structures present in the peptide, polypeptide, protein, glycoprotein, protein-DNA complex, protein-membrane complex and/or lipoprotein of binding multiligand receptors and activating an animal's immune system.

Preferably monomers and/or multimers of the peptide, polypeptide, protein, glycoprotein, protein-DNA complex, protein-membrane complex and/or lipoprotein in the immunogenic composition have dimensions in the range of 0.5 nm to 1000 μm, and more preferably, in the range of 0.5 nm to 100 μm, and even more preferably in the range of 1 nm to 5 μm, and even more preferably in the range of 3-2000 nm. Obviously, this range of dimensions is determined by the number of protein molecules per multimer, with a given number of amino-acid residues per protein molecule. Therefore, the dimensions are alternatively and/or additively expressed in terms of number of protein monomers per multimer.

In certain embodiments, tested is whether between 4-75% of the peptide, polypeptide, protein, glycoprotein, protein-DNA complex, protein-membrane complex and/or lipoprotein content of the composition is in a conformation comprising cross-beta structures. According to the invention, even though cross-beta structure enhances immunogenicity, the presence of too many cross-beta structures negatively influences immunogenicity. A cross-beta content between (and including) 4 and 75% is preferred. It is possible to determine the ratio between total cross-beta structure and total protein content. In a preferred embodiment, however, the cross-beta content within single proteins is determined. Preferably, individual proteins have a cross-beta content of between (and including) 4 and 75%, so that at least one epitope remains available for an animal's immune system. Most preferably, at least 70% of the individual proteins each have a cross-beta content of between (and including) 4 and 75%.

In certain embodiments, tested is whether the at least one cross-beta structure comprises a property allowing recognition, excision, processing and/or presentation of a T-cell epitope of the peptide, polypeptide, protein, glycoprotein, protein-DNA complex, protein-membrane complex and/or lipoprotein by an animal's immune system. Recognition of a cross-beta structure by a component of an animal's immune system, for instance by a multiligand receptor, such as but not limited to LRP, CD36, RAGE, SR-A, or LOX-1, results in (the initiation of) an immunogenic reaction against a peptide, polypeptide, protein, glycoprotein, protein-DNA complex, protein-membrane complex and/or lipoprotein of an immunogenic composition according to the invention (see for instance FIG. 11). It is therefore preferably tested whether a cross-beta structure of an immunogenic composition according to the invention has a desired (binding) property.

In certain embodiments, tested is whether a compound capable of specifically binding, recognizing, excising, processing and/or presenting a T-cell epitope of the peptide, polypeptide, protein, glycoprotein, protein-DNA complex, protein-membrane complex and/or lipoprotein is capable of specifically binding, recognizing, excising, processing and/or presenting the T-cell epitope. In principle, induction and/or administration of a cross-beta structure into a composition could result in a diminished availability of a T-cell epitope of interest. For instance, if a cross-beta structure is induced in a region of a peptide or protein wherein an epitope is present, the epitope is at risk of being shielded. The conformation of the epitope is also at risk of being disturbed. Alternatively, if a peptide sequence of a composition is coupled to a cross-beta containing peptide or protein, the coupling could take place at the site of an epitope of interest, thereby reducing its availability for an animal's immune system. In short, the availability of a T-cell epitope of interest for an animal's immune system could be diminished after an immunogenic composition has been provided with cross-beta structures. This is in certain embodiments, tested by determining whether a compound which is capable of specifically binding, recognizing, excising, processing and/or presenting a T-cell epitope of interest is still capable of binding, recognizing, excising, processing and/or presenting the T-cell epitope after the composition has been provided with cross-beta structure. If the compound is capable of specifically binding, recognizing, excising, processing and/or presenting the T-cell epitope, it shows that the epitope is still available for an animal's immune system. The compound for instance comprises an intracellular protease capable of excising the T-cell epitope from the primary amino acid sequence of an antigen. In certain embodiments, the compound comprises a component of a MHC complex. The MHC complex comprises either MHC-I and/or MHC-II. In certain embodiments, the compound comprises a T-cell or a T-cell receptor. The ability of an immunogenic composition comprising amino-acid sequences with cross-beta conformation, referred to as “cross-beta-antigens,” to induce (primary) T cell responses in vivo is preferably tested in vitro using T-cells isolated from immunized animals, for example mammals. For example, T cells are isolated from mice. In certain embodiments, T-cells from a human individual who has been exposed to an antigen such as a pathogen are used. Alternatively, activation of naïve T cells is analyzed upon isolation of T-cells from non-immunized animals, for example mammals, for example from mice or human individuals.

Several methods for T-cell isolation are known and commonly used in practice by persons skilled in the art. Preferably, T-cells are isolated from blood or splenocytes, for example from splenocytes isolated from immunized mammals, for example mice. In certain embodiments, non-human mammals, for example mice are immunized with antigen, preferably immunogenic compositions comprising cross-beta adjuvant and peptide, polypeptide, protein, glycoprotein, protein-DNA complex, protein-membrane complex and/or lipoprotein comprising at least one T-cell epitope and/or T-cell epitope motif, preferably once or twice, and cells are isolated preferably between 3 and 14 days after immunization. Preferably, spleen cell suspensions or peripheral blood mononuclear cells are used. Splenocytes are preferably isolated using cell strainers, preferably with a pore size of 100 μm. Preferably, erythrocytes are removed from the cell suspension, preferably by a centrifugation step using Ficoll, or by hemolysis, preferably with a hypotonic buffer, preferably composed of ammonium chloride, preferably at 0.15 mM, and potassium bicarbonate, preferably at 0.1 mM, and ethylendiaminetetaacetic acid, preferably at 0.01 mM.

Subsequently, isolated and washed T-cells are used for analysis of their response towards immunogenic compositions comprising cross-beta adjuvant and peptide, polypeptide, protein, glycoprotein, protein-DNA complex, protein-membrane complex and/or lipoprotein comprising at least one T-cell epitope motif. For example, such analyses are performed in an indirect way with antigen presenting cells included in the analyzed cell cultures, and/or directly by assessing responsiveness towards T-cell epitope motifs, for example using peptides of such motifs.

If the immunogenic composition appears to be capable of eliciting and/or stimulating a T-cell response, it shows that at least one T-cell epitope is still available for an animal's immune system.

In a preferred embodiment, at least two of the above mentioned tests are carried out. Of course, any combination of tests is possible. In certain embodiments, at least three of the above mentioned tests are carried out.

The invention thus provides a method for producing an immunogenic composition which is capable of activating T-cells and/or a T-cell response, the composition comprising at least one peptide, polypeptide, protein, glycoprotein, protein-DNA complex, protein-membrane complex and/or lipoprotein comprising a T-cell epitope and/or a T-cell epitope motif, the method comprising providing the composition with at least one cross-beta structure and determining: whether the degree of multimerization of the peptide, polypeptide, protein, glycoprotein, protein-DNA complex, protein-membrane complex and/or lipoprotein in the composition allows recognition, excision, processing and/or presentation of a T-cell epitope of the peptide, polypeptide, protein, glycoprotein, protein-DNA complex, protein-membrane complex and/or lipoprotein by an animal's immune system; whether between 4-75% of the peptide, polypeptide, protein, glycoprotein, protein-DNA complex, protein-membrane complex and/or lipoprotein content of the composition is in a conformation comprising cross-beta structures; whether the at least one cross-beta structure comprises a property allowing recognition, excision, processing and/or presentation of a T-cell epitope of the peptide, polypeptide, protein, glycoprotein and/or lipoprotein by an animal's immune system; and/or whether a compound capable of specifically binding, recognizing, excising, processing and/or presenting a T-cell epitope of the peptide, polypeptide, protein, glycoprotein, protein-DNA complex, protein-membrane complex and/or lipoprotein is capable of specifically binding, recognizing, excising, processing and/or presenting a T-cell epitope of the peptide.

In certain embodiments, it is determined whether monomers and/or multimers of the peptide, polypeptide, protein, glycoprotein, protein-DNA complex, protein-membrane complex and/or lipoprotein in the immunogenic composition have dimensions in the range of 0.5 nm to 1000 μm, and more preferably, in the range of 0.5 nm to 100 μm, and even more preferably in the range of 1 nm to 5 μm, and even more preferably in the range of 3-2000 nm. Obviously, this range of dimensions is determined by the number of protein molecules per multimer, with a given number of amino-acid residues per protein molecule. Therefore, the dimensions are alternatively and/or additively expressed in terms of number of protein monomers per multimer.

An animal comprises any animal having an immune system, preferably a mammal. In certain embodiments, the mammal is a human individual.

A protein-membrane complex is defined as a compound or composition comprising an amino acid sequence as well as a lipid molecule, and/or a fragment thereof, and/or a derivative thereof, for example assembled in a membrane and/or vesicle and/or liposome type of arrangement.

An immunogenic composition comprising at least one peptide, polypeptide, protein, glycoprotein, protein-DNA complex, protein-membrane complex and/or lipoprotein is defined herein as a composition comprising at least one amino acid sequence, which composition is capable of eliciting and/or enhancing an immune response in an animal, preferably a mammal, against at least part of the peptide, polypeptide, protein, glycoprotein, protein-DNA complex, protein-membrane complex and/or lipoprotein after administration of the immunogenic composition to the animal. The immune response may comprise a humoral immune response and/or a cellular immune response. The immune response need not be protective, therapeutic and/or capable of diminishing a consequence of disease. An immunogenic composition according to the invention is preferably capable of inducing and/or enhancing the formation of antibodies, and/or activating B-cells and/or T-cells which are capable of specifically binding an epitope of the peptide, polypeptide, protein, glycoprotein, protein-DNA complex, protein-membrane complex and/or lipoprotein.

In certain embodiments, it is determined whether a proteolytic system, for example the MHC antigen processing pathway, is capable of binding, recognizing, excising, processing and/or presenting a T-cell epitope of the peptide, polypeptide, protein, glycoprotein, protein-DNA complex, protein-membrane complex and/or lipoprotein in the context of either MHC-I and/or MHC-II.

In certain embodiments, it is determined whether the immunogenic composition and/or cross-beta structure is capable of specifically binding a cross-beta structure binding compound, preferably at least one compound selected from the group consisting of tPA, BiP, factor XII, fibronectin, hepatocyte growth factor activator, at least one finger domain of tPA, at least one finger domain of factor XII, at least one finger domain of fibronectin, at least one finger domain of hepatocyte growth factor activator, Thioflavin T, Thioflavin S, Congo Red, CD14, a multiligand receptor such as RAGE or CD36 or CD40 or LOX-1 or TLR2 or TLR4, a cross-beta-specific antibody, preferably cross-beta-specific IgG and/or cross-beta-specific IgM, IgIV, an enriched fraction of IgIV capable of specifically binding a cross-beta structure, Low density lipoprotein Related Protein (LRP), LRP Cluster II, LRP Cluster IV, Scavenger Receptor B-I (SR-BI), SR-A, chrysamine G, a chaperone, a heat shock protein, HSP70, HSP60, HSP90, gp95, calreticulin, a chaperonin, a chaperokine and a stress protein.

If the immunogenic composition appears to be capable of specifically binding such cross-beta binding compound, it shows that the immunogenic composition comprises a cross-beta structure which is capable of inducing and/or activating an animal's immune system.

Molecular chaperones are a diverse class of proteins comprising heat shock proteins, chaperonins, chaperokines and stress proteins, that are contributing to one of the most important cell defense mechanisms that facilitates protein folding, refolding of partially denatured proteins, protein transport across membranes, cytoskeletal organization, degradation of disabled proteins, and apoptosis, but also act as cytoprotective factors against deleterious environmental stresses. Individual members of the family of these specialized proteins bind non-native states of one or several or whole series or classes of proteins and assist them in reaching a correctly folded and functional conformation. Alternatively, when the native fold cannot be achieved, molecular chaperones contribute to the effective removal of misfolded proteins by directing them to the suitable proteolytic degradation pathways. Chaperones selectively bind to non-natively folded proteins in a stable non-covalent manner. To direct correct folding of a protein from a misfolded form to the required native conformation, mostly several chaperones work together in consecutive steps.

Chaperonins are molecular machines that facilitate protein folding by undergoing energy (ATP)-dependent movements that are coordinated in time and space by complex allosteric regulation. Examples of chaperones that facilitate refolding of proteins from a misfolded conformation to a native form are heat shock protein (hsp) 90, hsp60 and hsp70. Chaperones also participate in the stabilization of unstable protein conformers and in the recovery of proteins from aggregates. Molecular chaperones are mostly heat- or stress-induced proteins (hsps), that perform critical functions in maintaining cell homeostasis, or are transiently present and active in regular protein synthesis. Hsps are among the most abundant intracellular proteins. Chaperones that act in an ATP-independent manner are for example the intracellular small hsps, calreticulin, calnexin and extracellular clusterin. Under stress conditions such as elevated temperature, glucose deprivation and oxidation, small hsps and clusterin efficiently prevent the aggregation of target proteins. Interestingly, both types of hsps can hardly chaperone a misfolded protein to refold back to its native state. In patients with Creutzfeldt-Jakob, Alzheimer's disease and other diseases related to protein misfolding and accumulation of amyloid, increased expression of clusterin and small hsps has been seen. Molecular chaperones are essential components of the quality control machineries present in cells. Due to the fact that they aid in the folding and maintenance of newly translated proteins, as well as in facilitating the degradation of misfolded and destabilized proteins, chaperones are essentially the cellular sensors of protein misfolding and function. Chaperones are therefore the gatekeepers in a first line of defense against deleterious effects of misfolded proteins, by assisting a protein in obtaining its native fold or by directing incorrectly folded proteins to a proteolytic breakdown pathway. Notably, hsps are over-expressed in many human cancers. It has been established that hsps play a role in tumor cell metastasis, proliferation, differentiation, invasion, death, and in triggering the immune system during cancer.

One of the key members of the quality control machinery of the cell is the ubiquitous molecular chaperone hsp90. Hsp90 typically functions as part of large complexes, which include other chaperones and essential cofactors that regulate its function. Different cofactors seem to target hsp90 to different sets of substrates. However, the mechanism of hsp90 function in protein misfolding biology remains poorly understood.

Intracellular pathways that are involved in sensing protein misfolding comprise the unfolded protein response machinery (UPR) in the endoplasmic reticulum (ER). Accumulation of unfolded and/or misfolded proteins in the ER induces ER stress resulting in triggering of the UPR. Environmental factors can transduce the stress response, like for example changes in pH, starvation, reactive oxygen species. During these episodes of cellular stress, intracellular heat shock proteins levels increase to provide cellular protection. Activation of the UPR includes the attenuation of general protein synthesis and the transcriptional activation of the genes encoding ER-resident chaperones and molecules involved in the ER-associated degradation (ERAD) pathway. The UPR reduces ER stress by restoration of the protein-folding capacity of the ER. A key protein acting as a sensor of protein misfolding is the chaperone BiP (also referred to as grp78; Immunoglobulin heavy chain-binding protein/Endoplasmic reticulum luminal Ca²⁺-binding protein).

After testing of at least one immunogenic property of an immunogenic composition according to the invention, an immunogenic composition with a desired property is preferably selected. If a desired property, such as the availability of a T-cell epitope of interest, appears not to be present (anymore) after a composition comprising at least one peptide, polypeptide, protein, glycoprotein, protein-DNA complex, protein-membrane complex and/or lipoprotein has been provided with cross-beta structures, another batch of the same kind of composition is preferably provided with cross-beta structures and tested again. If needed, this procedure is repeated until an immunogenic composition with at least one desired property/properties is obtained.

In certain embodiments, an immunogenic composition is selected with a degree of multimerization of the peptide, polypeptide, protein, glycoprotein, protein-DNA complex, protein-membrane complex and/or lipoprotein which allows recognition, excision, processing and/or presentation of a T-cell epitope by an animal's immune system. Further provided is therefore a method hereof, further comprising selecting an immunogenic composition wherein the degree of multimerization of the peptide, polypeptide, protein, glycoprotein, protein-DNA complex, protein-membrane complex and/or lipoprotein in the composition allows recognition, excision, processing and/or presentation of a T-cell epitope of the peptide, polypeptide, protein, glycoprotein, protein-DNA complex, protein-membrane complex and/or lipoprotein by an animal's immune system.

In certain embodiments, an immunogenic composition is selected with a cross-beta content of between 4-75% so that the immunogenicity is enhanced, while at least one epitope remains available for an animal's immune system. The term immunogenicity is defined herein as the capability of a compound or a composition to activate an animal's immune system. Of course, if it is intended that an animal's immune system is, at least in part, directed against an epitope of interest, the epitope of interest should be available for the animal's immune system. Further provided is therefore a method hereof, further comprising selecting an immunogenic composition wherein between 4-75% of the peptide, polypeptide, protein, glycoprotein, protein-DNA complex, protein-membrane complex and/or lipoprotein content of the composition is in a conformation comprising cross-beta structures.

In yet another embodiment an immunogenic composition is selected which comprises a cross-beta structure having a binding property which allows (the initiation of) an immunogenic reaction against a peptide, polypeptide, protein, glycoprotein, protein-DNA complex, protein-membrane complex and/or lipoprotein of an immunogenic composition according to the invention. Further provided is therefore a method hereof, further comprising selecting an immunogenic composition which comprises a cross-beta structure which is capable of specifically binding a cross-beta structure binding compound, preferably tPA, BiP, factor XII, fibronectin, hepatocyte growth factor activator, at least one finger domain of tPA, at least one finger domain of factor XII, at least one finger domain of fibronectin, at least one finger domain of hepatocyte growth factor activator, Thioflavin T, Thioflavin S, Congo Red, CD14, a multiligand receptor such as RAGE or CD36 or CD40 or LOX-1 or TLR2 or TLR4, a cross-beta-specific antibody, preferably cross-beta-specific IgG and/or cross-beta-specific IgM, IgIV, an enriched fraction of IgIV capable of specifically binding a cross-beta structure, Low density lipoprotein Related Protein (LRP), LRP Cluster II, LRP Cluster IV, Scavenger Receptor B-I (SR-BI), SR-A, chrysamine G, a chaperone, a heat shock protein, HSP70, HSP60, HSP90, gp95, calreticulin, a chaperonin, a chaperokine, and/or a stress protein.

In yet another embodiment, an immunogenic composition is selected whereby a proteolytic system, for example the MHC antigen processing pathway, is capable of recognizing, binding, excising, processing and/or presenting a T-cell epitope of the peptide, polypeptide, protein, glycoprotein, protein-DNA complex, protein-membrane complex and/or lipoprotein in the context of either MHC-I and/or MHC-II.

### BRIEF DESCRIPTION OF THE DRAWINGS

FIG. 1. Coomassie stained SDS-PA gel and Western blot with nE2 and nE2-FLAG-His. Lane 1: Coomassie nE2-FLAG-His (non-reducing); Lane 2: Western blot nE2-FLAG-His (non-reducing; anti-FLAG antibody); Lane 3: Coomassie nE2 in culture medium (non-reducing); Lane 4: Western blot nE2 in culture medium (non-reducing; mix of 3 monoclonal antibodies); Lane 5: Coomassie nE2 dialyzed to PBS and concentrated (non-reducing); Lane 6: Western blot nE2 dialyzed to PBS and concentrated (non-reducing; mix of 3 monoclonal antibodies); Lane 7: Coomassie nE2-FLAG-His (reducing); Lane 8: Western blot nE2-FLAG-His (reducing; anti-FLAG antibody); Lane 9: Coomassie nE2 in culture medium (reducing); Lane 10: Western blot nE2 in culture medium (reducing; mix of 3 monoclonal antibodies); Lane 11: molecular weight marker.

FIG. 2. Structure analyses of non-treated E2 and misfolded E2. E2 expressed in Sf9 cells and in cell culture medium was dialyzed against PBS and approximately tenfold concentrated, designated as nE2. Misfolded cross-beta E2 (cE2) was obtained by cyclic heating of nE2 (see text for details). A. Thioflavin T fluorescence enhancement assay with nE2 and cE2 at 100 μg/ml. Standard is 100 μg/ml dOVA. The fluorescence measured with dOVA standard is arbitrarily set to 100%. Buffer control was PBS. B. tPA/plasminogen chromogenic activation assay with nE2 and cE2 at 12.5 and 50 μg/ml in the assay. C. Transmission electron microscopy image of nE2. The scale bar is given in the image. D. TEM image of cE2.

FIG. 3. Transmission electron microscopy image of misfolded ovalbumin at 1 mg/ml.

FIG. 4. Coomassie-stained gel and Western blot with the H5 variants nH5-1, nH5-2, CH5-A, CH5-B. A. Non reducing SDS NuPage gel applied with nH5-1, nH5-2, CH5-A, CH5-B originating from H5-FLAG-His of H5N1 strain A/HK/156/97. Marker: 6 μl/lane, Precision Plus Protein Dual Color Standards, BioRad, Cat.#161-0374. Gel:NuPage 4-12% Bis-Tris Gel, 1.0 mm×10 well, Invitrogen, Cat.# NP0321BOX. M=Marker; nH5-1, 2 μg; nH5-2, 0.66 μg; CH5-A, 2 μg; CH5-B, 2 μg. B. Western blot with the H5 variants nH5-1, nH5-2, CH5-A, CH5-B, analyzed with peroxidase-labeled anti-FLAG antibody. In each indicated lane 30 ng H5 is loaded. H5 is of H5N1 strain A/Hong kong/156/97. Marker: 6 μl/lane, Precision Plus Protein Dual Color Standards, BioRad, Cat.#161-0374. Gel:NuPage 4-12% Bis-Tris Gel, 1.0 mm×10 well, Invitrogen, Cat.# NP0321BOX.

FIG. 5. Size exclusion chromatography analysis with non-treated H5 and H5 subjected to a misfolding procedure. The non-treated H5-FLAG-His, nH5-1, and this sample incubated at 37° C. with 100 mM DTT (CH5-B), originating from H5 of H5N1 strain A/HK/156/97, were subjected to a SEC column for analysis of the size distribution of H5 multimers, observed on Coomassie-stained SDS-PA gels applied with non-reducing conditions.

FIG. 6. Identification of soluble oligomers in H5 samples, of H5N1 strain A/HK/156/97, using ultracentrifugation. The H5 samples originating from H5N1 strain A/HK/156/97, as indicated in the graphs, were subjected to centrifugation for 10 minutes at 16,000*g (nH5-2, indicated with “16k*g”), or for 60 minutes at 100,000*g (nH5-2, CH5-A, CH5-B, indicated with “100k*g”). A. Protein concentration in the three H5 samples before and after centrifugation at the indicated times/g-forces. Relative concentrations are given for comparison. B. ThT fluorescence of four-fold diluted H5 samples before and after centrifugation at the indicated times/g-forces.

FIG. 7. Analysis of cross-beta structure in H5-FLAG-His samples. The H5 originates from H5N1 strain A/HK/156/97 and comprises a C-terminal FLAG-tag, followed by a His-tag. A. ThT fluorescence of the two non-treated H5 forms (nH5-1, nH5-2) and the two forms obtained after applying different misfolding procedures (CH5-A, CH5-B), tested at the indicated concentrations. Standard: 100 μg/ml cross-beta dOVA; fluorescence arbitrarily set to 100%. B. Congo red fluorescence of the non-treated H5 forms nH5-1 and CH5-A, CH5-B, tested at the indicated concentrations. Standard: 100 μg/ml cross-beta dOVA; fluorescence arbitrarily set to 100%. C. tPA/plasminogen activation assay using chromogenic plasmin substrate and depicted H5 solutions at the indicated concentrations. Standard: 40 μg/ml cross-beta dOVA; activity arbitrarily set to 100%. D. Transmission electron microscopy image of non-treated H5 form nH5-1. The bar indicates the scale of the image. E. Transmission electron microscopy image of nH5-2. F. Transmission electron microscopy image of CH5-A obtained after applying a misfolding procedure, as indicated in the text.

FIG. 8. Coomassie stained gel with a concentration series of H5 of H5N1 A/Vietnam/1203/04, under reduced and non-reduced conditions. H5 protein of H5N1 A/VN/1203/04 under reducing (sample 1-4) and non-reducing (sample 5-8) conditions. M=marker, lane 1, 5=4 μg H5, lane 2, 6=2 μg H5, lane 3, 7=1 μg H5, lane 4, 8=0.5 μg H5. Marker: 6 μl/lane, Precision Plus Protein Dual Color Standards, BioRad, Cat.#161-0374. Gel:NuPage 4-12% Bis-Tris Gel, 1.0 mm×10 well, Invitrogen, Cat.# NP0321BOX.

FIG. 9. TEM images of non-treated H5 of H5N1 A/VN/1203/04, and accompanying misfolded H5 variants CH5-1-4, comprising cross-beta. TEM analysis of nH5 (A.) shows amorphous aggregates. The incidence of aggregates is reduced to ˜5 aggregates/mesh in CH5-1 (B.), but the aggregates are larger in size, more dense and the morphology is changed compared to nH5. A high incidence of dense aggregates was observed in CH5-2 (C.). In the preparation of CH5-3 (D.), aggregates of similar morphology compared to CH5-2 were observed, but with reduced incidence. Lower aggregate count and dissimilar morphology of aggregates was observed for CH5-4 (E.).

FIG. 10. ThT and Congo red fluorescence enhancement measurements for non-treated and misfolded H5 (recombinantly produced H5 of H5N1 strain A/Vietnam/1203/04). Thioflavin T (A.) and Congo red fluorescence enhancement measurements (B.) of H5 show elevated fluorescence for the preparations CH5-1, CH5-2 and CH5-3 that were subjected to conditions favoring protein misfolding. Reduction in fluorescence intensity was observed in preparation CH5-4. The preparation CH5-1 was slightly turbid with some visible precipitates after heat treatment, which could explain the high standard deviation. C. tPA mediated plasminogen activation assay of non-treated and misfolded H5 variants originating from recombinantly produced H5 of H5N1 strain A/VN/1203/04. CH5-2 (150% of standard) and CH5-3 (200% of standard) are more potent cofactors for the activation of tPA/plasminogen compared to the starting material of nH5 (140% of standard). Lower activations were observed with CH5-1 (50% of standard) and CH5-4 (37% of standard) compared to the starting material. Substantial activation is observed with the starting material nH5, indicating that this H5 preparation already harbors misfolded proteins to some extent.

FIG. 11. Schematic overview of humoral immune response and cellular immune response.

FIG. 12. SDS-PAGE analysis with non-reducing conditions, with various OVA samples. For preparation of various OVA and description of the analysis see text.

FIG. 13. Enhancement of Thioflavin T fluorescence under influence of various OVA forms. Various forms of dOVA comprise cross-beta structure (see also text and Table 4 for further description).

FIG. 14. Enhancement of Sypro Orange fluorescence under influence of various OVA forms. It is seen that dOVA forms have increased cross-beta structure (see also text and Table 5).

FIG. 15. tPA-mediated plasminogen activation assay with OVA samples. tPA activation potential was determined at the indicated concentration of 80, 25 and 10 μg/ml OVA. Right and left panel are graphs of two experiments. It is seen that cross-beta structure inducing methods induces cross-beta structure (for further details see text and Table 6).

FIG. 16. Binding of Fn F4-5 to various forms of OVA, as determined in an ELISA with immobilized OVA. It is seen that Fn 4-5 has increased binding to dOVA forms compared to nOVA. See also text and Table 7.

FIG. 17. IL-2 secretion by DO11.10 after co-culture with OVA-pulsed BMDC. Immature 1×10⁵ BMDC, pulsed with the indicated amount of OVA for 24 hours, were co-cultured with 1×10⁵ DO11.10 T cells. Activation is determined by the amount of IL-2 that is released by DO11.10 T cells after 24 hours.

FIG. 18. Proliferation of OT-II after co-culture with OVA-pulsed BMDC. Activation is measured by incorporation of 3H-thymidine. It is seen that dOVA1, 2 and 3 are potent inducers of T cells, with dOVA-2 being the most potent and in the order of dOVA-1>dOVA-3>dOVA-2>nOVA.

FIG. 19. Activation of CD8 naive T cells (OT-I cells from transgenic mice) by OVA samples after successful processing and presentation by APCs. Activation is determined by measuring the proliferative potential (3H-thymidine incorporation).

FIG. 20. anti-OVA IgG after immunization with structurally different OVAs. 13 C57BL-6 mice were immunized on day 0, 7, 14 and 21 with 5 μg OVA subcutaneously. At day 25 serum was collected and total IgG was determined by ELISA. Results are expressed as Log¹⁰ of the OD50+/−SEM. See also Table 11.

FIG. 21. OVA-specific T cell response after immunization with OVA samples. Splenocytes were isolated on day 30 from mice immunized with the indicated OVAs and analyzed for (A) pentamer SIINFEKLL-MHCI-staining, (B) IFN γ release by ELISPOT, and (C) IL-5 release by ELISPOT by T cells was analyzed.

FIG. 22. T cell response after immunization with OVA samples. ELISPOT analysis of IFN γ (A) and IL-5 (B) released by T cells in response to nOVA and dOVA (at the indication concentration (x-axis)) antigen uptake, processing and presentation by APCs in isolated splenocytes cultured ex vivo in the presence of nOVA.

FIG. 23. Tumor growth after immunization with OVA samples and challenge with OVA expressing EG7 tumor cells. Ten mice in each group were inoculated with 5×10e5 tumor cells in both the left and the right flank. Tumor number (A) and tumor index (B and C) [(ab)e0.5, in which a and b are the longest and shortest diameter of the tumors) was determined. ***p<0.001; **p<0.005; *p<0.05.

FIG. 24. Correlation IgG response and tumor growth. Titers of nOVA, dOVA-1, dOVA-2, dOVA-3, dOVA-4 and nOVA+CFA-immunized mice were determined on day 25 and the average log¹⁰ titer was determined for each group (n=10). Tumor cells were inoculated on day 28 and tumor growth was monitored. The average of the tumor growth of the mice in each group was determined on day 7, 15 and 21. The correlation between the average log¹⁰ titers (Y-axis) and average tumor growth (X-axis) in each group is shown. Correlation day 15 R²: 0.9656 with p-value 0.005; day 21 R²: 0.9268 with p-value 0.0021.

FIG. 25. Correlation IgG response and tumor growth. A. Titers of nOVA, dOVA-1, dOVA-2, dOVA-3, dOVA-4 and nOVA+CFA-immunized mice were determined on day 25 and the average log¹⁰ titer was determined for each group (n=10). Tumor cells were inoculated on day 28 and tumor growth was monitored. The average of the tumor growth of the mice in each group was determined on day 7, 15 and 21. The correlation between the average log¹⁰ titers (Y-axis) and average tumor growth (X-axis) in each group is shown. B. Sequence of Hemagglutinin 5 protein (H5) of H5N1 virus strain A/Hong kong/156/97 (A/HK/156/97) with a C-terminal FLAG tag and His tag.

FIG. 26. SDS-PAGE analysis (Coomassie staining) under reducing (left) or non-reducing conditions (right), with nH5 samples. Left: Arrows indicate HA0, uncleaved H5, or HA1 and HA2, the processed form of H5. Right: Black arrow indicates monomeric H5 and white arrow indicates multimeric forms of H5. Lane 1, 2, and 3, 4 μg, 2 μg, and 1 μg of one H5 batch, lane 4, empty, lane 5, 6 and 7, 2.75 μg, 2 μg, and 0.5 μg of another batch of H5. Lane 8 and 9 are empty. Lane 10 contains molecular weight marker. Inset shows size of the molecular weight marker (kDa).

FIG. 27. Western blot analysis (anti-FLAG antibody) under reducing (left) or non-reducing conditions (right), with nH5 samples. Left: Arrows indicate HA0, uncleaved H5 (upper arrow), or HA2, the processed form of H5 (lower arrow) with the FLAG-tag. Right: Black arrow indicates monomeric H5 and white arrow indicates dimeric, trimeric forms of H5. Lane 1, and 2, 10 ng and 5 ng, of one batch of H5, lane 3 and 4 10 ng and 5 ng of another batch, lane 5, and 6, 10 and 5 ng of a third batch. Lane 7, 8 and 9 are empty. Lane 10 contains molecular weight marker. Inset shows size of the molecular weight marker.

FIG. 28. SDS-PAGE analysis (Coomassie staining) of 8.96 μg and 3.5 μg nBSA (lane 4 and 6), 8.96 μg hdBSA (lane 5).

FIG. 29. TEM analysis of nH5-dOVA. On average, three types of aggregates are observed. The few relatively large and dense aggregates have the appearance of clustered beads, which arrange amorphously with approximate dimensions of 200-500 nm×2000 nm. The smaller and less dense aggregates also seen composed of bead like arrangements of molecules, now clustered with less bead “monomers,” 50-100 nm×200-500 nm in size. The smallest aggregates are seemingly the bead “monomers” of which the larger aggregates are built up. The radius is approximately 10-20 nm.

FIG. 30. ThT fluorescence enhancement analysis of H5 samples. The total protein concentration in the ThT fluorescence enhancement assay is 6.25 μg/ml for nH5, 50 μg/ml for nH5-hdBSA, 33.9 μg/ml for dOVA and 50 μg/ml for nH5-dOVA, whereas the nH5 concentration is constant at 6.25 μg/ml.

FIG. 31. T cell activation analysis by IFNγ-ELISPOT. Activation is indicated as the number of spot forming units (SFU) per number of seeded cells (2×10e5). Splenocytes with the T cells isolated from the indicated groups (A, nH5, B, cbH5 [nH5+dOVA and hdBSA] or C, placebo), were stimulated with 10 μg/ml nH5 or peptides derived from the sequence of H5. The result shows that mice immunized with H5 in combination with dOVA and hdBSA have increased number of H5 specific T cells as compared to mice immunized with H5 alone (122 SFU vs 68 SFU, p=0.0017). H5-specific activation of T cells is also demonstrated with two H5 specific peptides as activation is seen with these peptides of T cells isolated from mice immunized with nH5 (or nH5 in combination with hdBSA and dOVA) as compared to placebo. The fact that, using these peptides as stimulus, there is no increase in the number of SFU between the T cells isolated from group A vs. group B suggests that other epitopes are present in the group immunized with nH5 in combination with dOVA and hdBSA that may contribute to the increased number of SFU seen with H5 protein as stimulant in the ELISPOT assay.

FIG. 32. SEC elution pattern of dH5-0 and melting curve of cdH5-0, as determined by measuring Sypro Orange fluorescence during increasing temperature. A. SEC elution pattern of dH5-0. Approximately 65% of the dH5-0 elutes as a 33 kDa protein. B. Melting curve of cdH5-0. Half of the cdH5-0 molecules are molten at T=52.5° C. FIG. 33. H5 forms analyzed on SDS-PA gel under reducing and non-reducing conditions. A. Lane M, marker with indicated molecular weights in kDa; lane 1 and 7, dH5-0; lane 2 and 8, cdH5-0; lane 3 and 9, fdH5-0; lane 4 and 10, dH5-I; lane 5 and 11, dH5-II; lane 6 and 12, dH5-III. Samples in lanes 1-6 are pre-incubated in non-reducing buffer (disulphide bonds stay intact), samples 7-12 are pre-heated in buffer comprising reducing agent dithiothreitol (DTT). B. SDS-PAGE analysis with non-reducing conditions, with various H5 samples, before/after ultracentrifugation.

FIG. 34. Enhancement of Thioflavin T fluorescence (A.) and Sypro orange fluorescence (B.) under influence of various H5 forms.

FIG. 35. Binding of Fn F4-5 to various forms of H5, as determined in an ELISA with immobilized H5.

FIG. 36. Binding of tPA to various structural variants of H5 and results of a tPA-mediated plasminogen activation assay with non-treated, misfolded and ultracentrifuged H5 samples, determined at 50 μg/ml H5. (A-D) In an ELISA the binding of tPA to H5 forms was tested. To avoid putative binding of the tPA kringle 2 domain to exposed lysine and arginine residues, the binding experiment is performed in the presence of an excess ε-amino caproic acid. In A, B and D, binding of tPA is shown, whereas in C binding of the negative control K2P tPA, which lacks the cross-beta binding finger domain, is shown. (E) tPA/Plg activating potential was tested for the six different H5 forms. The activating potential of cross-beta ovalbumin standard at 30 μg/ml is set to 100%; at 10 and 50 μg/ml, tPA/plg activation is 100% and 85%, respectively. H5 samples are all tested at 50 μg/ml.

FIG. 37. Antibody response of mice immunized with various forms of H5. Anti-H5 specific antibodies induced by immunization with various forms of H5 were determined using an ELISA. Mice immunized with dH5-0, cdH5-0 and fdH5-0 have significant higher titers compared to dH5-I, dH5-II and dH5-III (* indicates p<0.05). FIG. 38. T cell response of mice immunized with various forms of H5. Splenocytes were isolated on day 41 from mice immunized with the indicated H5 forms and analyzed for IFN γ release by ELISPOT. The method was identical to that used for the ELISPOT analyses with OVA, except that cdH5-0 (“cnH5”) was used as stimulus. It is seen that immunogenic composition with H5 induce a T cell response. All H5 immunogenic composition comprising cross-beta structure induce a T cell response with some differences in induction capacity, being dH5-0 (“nH5”) the strongest.

### DETAILED DESCRIPTION OF THE INVENTION

Methods disclosed herein are particularly suitable for selecting, from a plurality of immunogenic compositions, one or more immunogenic compositions having a greater chance of being capable of eliciting and/or stimulating a protective prophylactic immune response and/or a therapeutic immune response in vivo, as compared to the other immunogenic compositions of the plurality of immunogenic compositions. One or more immunogenic compositions are selected which appear to have a desired property in any of the aforementioned tests. Further provided is therefore an in vitro method for selecting, from a plurality of immunogenic compositions comprising at least one cross-beta structure and at least one peptide and/or polypeptide and/or protein and/or glycoprotein and/or protein-DNA complex and/or protein-membrane complex and/or lipoprotein with a T-cell epitope or a T-cell epitope motif, one or more immunogenic compositions having a higher chance of being capable of eliciting and/or stimulating a protective prophylactic cellular immune response and/or a therapeutic cellular immune response in vivo, as compared to the other immunogenic compositions of the plurality of immunogenic compositions, the method comprising: selecting, from the plurality of immunogenic compositions, an immunogenic composition: wherein the degree of multimerization of the peptide, polypeptide, protein, glycoprotein, protein-DNA complex, protein-membrane complex and/or lipoprotein in the composition allows recognition, excision, processing and/or presentation of a T-cell epitope of the peptide, polypeptide, protein, glycoprotein, protein-DNA complex, protein-membrane complex and/or lipoprotein by an animal's immune system; wherein between 4-75% of the peptide, polypeptide, protein, glycoprotein, protein-DNA complex, protein-membrane complex and/or lipoprotein content of the composition is in a conformation comprising cross-beta structures; which comprises a cross-beta structure which is capable of specifically binding a cross-beta structure binding compound, preferably tPA, BiP, factor XII, fibronectin, hepatocyte growth factor activator, at least one finger domain of tPA, at least one finger domain of factor XII, at least one finger domain of fibronectin, at least one finger domain of hepatocyte growth factor activator, Thioflavin T, Thioflavin S, Congo Red, CD14, a multiligand receptor such as RAGE or CD36 or CD40 or LOX-1 or TLR2 or TLR4, a cross-beta-specific antibody, preferably cross-beta-specific IgG and/or cross-beta-specific IgM, IgIV, an enriched fraction of IgIV capable of specifically binding a cross-beta structure, Low density lipoprotein Related Protein (LRP), LRP Cluster II, LRP Cluster IV, Scavenger Receptor B-I (SR-BI), SR-A, chrysamine G, a chaperone, a heat shock protein, HSP70, HSP60, HSP90, gp95, calreticulin, a chaperonin, a chaperokine and/or a stress protein; and/or whether a compound capable of specifically binding, recognizing, excising, processing and/or presenting a T-cell epitope of the peptide, polypeptide, protein, glycoprotein, protein-DNA complex, protein-membrane complex and/or lipoprotein is capable of specifically binding, recognizing, excising, processing and/or presenting the T-cell epitope.

In certain embodiments, it is determined whether a proteolytic system, for example, the MHC antigen processing pathway, is capable of recognizing, binding, excising processing and/or presenting a T-cell epitope of the peptide, polypeptide, protein, glycoprotein, protein-DNA complex, protein-membrane complex and/or lipoprotein in the context of either MHC-I and/or MHC-II.

A composition comprising at least one peptide, polypeptide, protein, glycoprotein, protein-DNA complex, protein-membrane complex and/or lipoprotein is provided with at least one cross-beta structure in various ways. In certain embodiments, the cross-beta structure is induced in at least part of the peptide, polypeptide, protein, glycoprotein, protein-DNA complex, protein-membrane complex and/or lipoprotein. Various methods for inducing a cross-beta structure are known in the art. For instance, the peptide, polypeptide, protein, glycoprotein, protein-DNA complex, protein-membrane complex and/or lipoprotein is at least in part misfolded. In certain embodiments, an immunogenic composition comprising at least one peptide, polypeptide, protein, glycoprotein, protein-DNA complex, protein-membrane complex and/or lipoprotein is subjected to a cross-beta inducing procedure. The cross-beta inducing procedure preferably comprises a change of pH, salt concentration, reducing agent concentration, temperature, buffer and/or chaotropic agent concentration. A method hereof, wherein at least one peptide, polypeptide, protein, glycoprotein, protein-DNA complex, protein-membrane complex and/or lipoprotein is subjected to a cross-beta inducing procedure, preferably a change of pH, salt concentration, reducing agent concentration, temperature, buffer and/or chaotropic agent concentration, is therefore also provided. Non-limiting examples of cross-beta inducing procedures are heating, chemical treatments with e.g., high salts, acid or alkaline materials, pressure and other physical treatments. A preferred manner of introducing cross-beta structures in an antigen is by one or more treatments, either in combined fashion or sequentially, of heating, freezing, reduction, oxidation, glycation pegylation, sulphatation, exposure to a chaotropic agent (the chaotropic agent preferably being urea or guanidinium-HCl), phosphorylation, partial proteolysis, chemical lysis, preferably with HCl or cyanogen bromide, sonication, dissolving in organic solutions, preferably 1,1,1,3,3,3-hexafluoro-2-propanol and trifluoroacetic acid, or a combination thereof.

In certain embodiments, the immunogenic composition comprising at least one peptide, polypeptide, protein, glycoprotein, protein-DNA complex, protein-membrane complex and/or lipoprotein is coupled to a cross-beta comprising compound. For instance, the peptide, polypeptide, protein, glycoprotein, protein-DNA complex, protein-membrane complex and/or lipoprotein is linked to a peptide or protein comprising a cross-beta structure. It is, however, also possible to administer a cross-beta comprising compound to a composition according to the invention, without linking the cross-beta comprising compound to the peptide, polypeptide, protein, glycoprotein, protein-DNA complex, protein-membrane complex and/or lipoprotein. Preferably the cross-beta comprising compound is an otherwise inert compound. “Inert” is defined as not eliciting and/or stimulating an undesired immune response or another unwanted biochemical reaction in a host, at least not to an unacceptable degree, preferably only to a negligible degree.

A cross-beta structure comprising compound may be added to a composition by itself, but it is also useful to use the cross-beta structure comprising compound as a carrier to which elements of the infectious agent(s) and/or antigen(s) of an immunogenic composition according to the invention are linked. This linkage can be provided through chemical linking (direct or indirect) or, for instance, by expression of the relevant antigen(s) and the cross-beta comprising compound as a fusion protein. In both cases linkers between the two may be present. In both cases dimers, trimers and/or multimers of the antigen (or one or more epitopes of a relevant antigen) may be coupled to a cross-beta comprising compound. However, normal carriers comprising relevant epitopes or antigens coupled to them may also be used. The simple addition of a cross-beta comprising compound will enhance the immunogenicity of such a complex. This is more or less generally true. An immunogenic composition according to the invention may typically comprise a number or all of the normal constituents of an immunogenic composition (in particular a vaccine), supplemented with a cross-beta structure (conformation) comprising compound.

In a preferred embodiment the cross-beta structure comprising compound is itself a vaccine component (i.e., derived from an infectious agent and/or antigen against which an immune response is desired).

An immunogenic composition hereof may be used for the preparation of a vaccine. A method hereof, further comprising producing a vaccine comprising the selected immunogenic composition, is therefore also herewith provided. Preferably, a prophylactic and/or therapeutic vaccine is produced. In certain embodiments, a subunit vaccine is produced.

In certain embodiments, an immunogenic composition which is produced and/or selected with a method hereof is used as a vaccine. Preferably, no other carriers, adjuvants and/or diluents are necessary because of the presence of cross-beta structures. However, if desired, such carriers, adjuvants and/or diluents may be administered to the vaccine composition at will. Further provided is therefore a use of an immunogenic composition produced and/or selected with a method hereof as a vaccine, preferably as a prophylactic and/or therapeutic vaccine. In certain embodiments, the vaccine comprises a subunit vaccine.

Further provided is an immunogenic composition selected and/or produced with a method hereof. The immunogenic composition preferably comprises a vaccine, more preferably a prophylactic and/or therapeutic vaccine. An immunogenic composition according to the invention is particularly suitable for the preparation of a vaccine for the prophylaxis and/or treatment of a disorder caused by a pathogen, tumor, cardiovascular disease, atherosclerosis, amyloidosis, autoimmune disease, graft-versus-host rejection and/or transplant rejection. A use of an immunogenic composition according to the invention for the preparation of a vaccine for the prophylaxis and/or treatment of a disorder caused by a pathogen, tumor, cardiovascular disease, atherosclerosis, amyloidosis, autoimmune disease, graft-versus-host rejection and/or transplant rejection is therefore also herewith provided.

Further provided are uses of such immunogenic compositions for at least in part preventing and/or counteracting such disorders. One embodiment provides a method for at least in part preventing and/or counteracting a disorder caused by a pathogen, tumor, cardiovascular disease, atherosclerosis, amyloidosis, autoimmune disease, graft-versus-host rejection and/or transplant rejection, comprising administering to a subject in need thereof a therapeutically effective amount of an immunogenic composition according to the invention. The animal is preferably a human individual.

A method hereof is particularly suitable for producing and/or selecting an immunogenic composition with desired, preferably improved, immunogenic properties. It is, however, also possible to perform a method hereof for improving existing immunogenic compositions. Further provided is therefore a method for improving an immunogenic composition, the composition comprising at least one peptide, polypeptide, protein, glycoprotein, protein-DNA complex, protein-membrane complex and/or lipoprotein comprising a T-cell epitope and/or a T-cell epitope motif, the method comprising providing the composition with at least one cross-beta structure and determining: whether the degree of multimerization of the peptide, polypeptide, protein, glycoprotein, protein-DNA complex, protein-membrane complex and/or lipoprotein in the composition allows recognition, excision, processing and/or presentation of a T-cell epitope of the peptide, polypeptide, protein, glycoprotein, protein-DNA complex, protein-membrane complex and/or lipoprotein by an animal's immune system; whether between 4-75% of the peptide, polypeptide, protein, glycoprotein, protein-DNA complex, protein-membrane complex and/or lipoprotein content of the composition is in a conformation comprising cross-beta structures; whether the at least one cross-beta structure comprises a property allowing recognition, excision, processing and/or presentation of a T-cell epitope of the peptide, polypeptide, protein, glycoprotein and/or lipoprotein by an animal's immune system; and/or whether a compound capable of specifically binding, recognizing, excising, processing and/or presenting a known T-cell epitope of the peptide, polypeptide, protein, glycoprotein, protein-DNA complex, protein-membrane complex and/or lipoprotein is capable of specifically binding, recognizing, excising, processing and/or presenting the T-cell epitope.

In certain embodiments, a method hereof is provided, wherein the T-cell epitope is a CTL epitope. In another preferred embodiment, a method hereof is provided, wherein the T-cell epitope is a T-helper cell epitope.

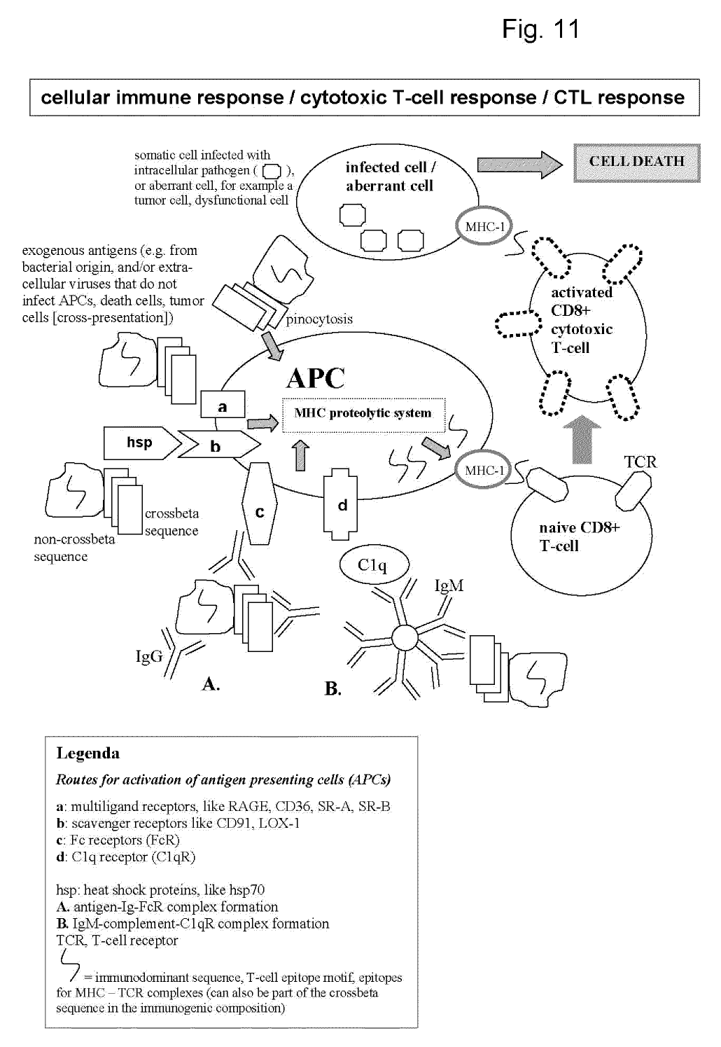

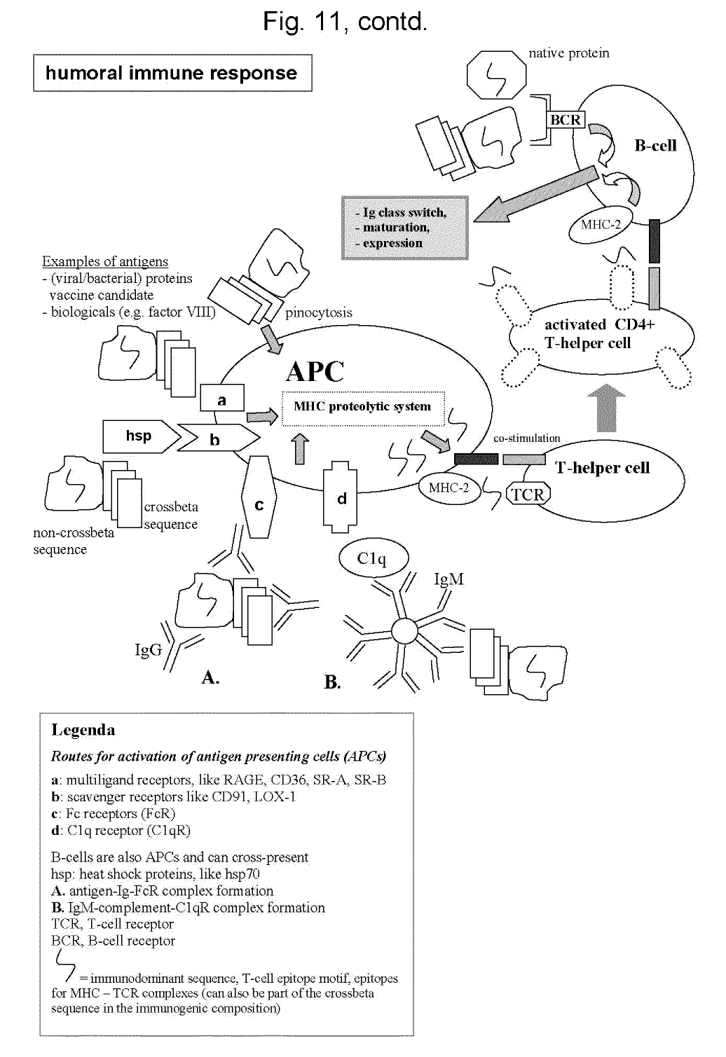

A method hereof is particularly suitable for producing and/or selecting an immunogenic composition which is capable of eliciting and/or stimulating a humoral and/or cellular immune response. For a schematic overview of a humoral and cellular immune response, reference is made to FIG. 11. In certain embodiments, a method hereof is used for producing and/or selecting an immunogenic composition which is specifically adapted for eliciting and/or stimulating a cellular immune response. In certain embodiments, a method hereof is used for producing and/or selecting an immunogenic composition which is specifically adapted for avoiding a cellular immune response. In certain embodiments, a method hereof is used for producing and/or selecting an immunogenic composition which is specifically adapted for eliciting and/or stimulating both a cellular and a humoral immune response. In certain embodiments, a method hereof is used for producing and/or selecting an immunogenic composition which is specifically adapted for eliciting and/or stimulating a humoral immune response.

In order to produce and/or select a composition comprising a peptide, polypeptide, protein, glycoprotein, protein-DNA complex, protein-membrane complex and/or lipoprotein which is specifically adapted for avoiding a cellular immune response, further provided is a method for producing an immunogenic composition comprising at least one peptide, polypeptide, protein, glycoprotein, protein-DNA complex, protein-membrane complex and/or lipoprotein, the method comprising: determining whether a peptide, polypeptide, protein, glycoprotein, protein-DNA complex, protein-membrane complex and/or lipoprotein lacks a T-cell epitope motif; selecting a peptide, polypeptide, protein, glycoprotein, protein-DNA complex, protein-membrane complex and/or lipoprotein lacking a T-cell epitope motif; providing a composition comprising the selected peptide, polypeptide, protein, glycoprotein, protein-DNA complex, protein-membrane complex and/or lipoprotein; and providing the composition with at least one cross-beta structure.

In order to produce and/or select a (candidate) composition comprising a peptide, polypeptide, protein, glycoprotein, protein-DNA complex, protein-membrane complex and/or lipoprotein which is specifically adapted for avoiding a cellular immune response, further provided is a method for producing an immunogenic composition, comprising determining: whether the degree of multimerization of the peptide, polypeptide, protein, glycoprotein, protein-DNA complex, protein-membrane complex and/or lipoprotein in the composition does not, or to an acceptable extent, allow recognition, excision, processing and/or presentation of a T-cell epitope of the peptide, polypeptide, protein, glycoprotein, protein-DNA complex, protein-membrane complex and/or lipoprotein by an animal's immune system; whether less than 4% of the peptide, polypeptide, protein, glycoprotein, protein-DNA complex, protein-membrane complex and/or lipoprotein content of the composition is in a conformation comprising cross-beta structures; whether the at least one cross-beta structure comprises a property which does not, or to an acceptable extent, allow recognition, excision, processing and/or presentation of a T-cell epitope of the peptide, polypeptide, protein, glycoprotein and/or lipoprotein by an animal's immune system; and/or whether a compound capable of specifically recognizing, binding, excising, processing and/or presenting a T-cell epitope of the peptide, polypeptide, protein, glycoprotein, protein-DNA complex, protein-membrane complex and/or lipoprotein is not, or to an acceptable extent, capable of specifically recognizing, binding, excising, processing and/or presenting the T-cell epitope.

The properties are preferably compared with a reference composition. When at least one of the properties appears to be more favorable as compared to the reference composition, the (candidate) composition is preferably used instead of the reference composition.

In order to produce and/or select an immunogenic composition which is suitable for eliciting a humoral immune response, a method hereof preferably comprises the following step: determining whether an antibody or a functional fragment or a functional equivalent thereof, capable of specifically binding an epitope of the peptide, polypeptide, protein, glycoprotein, protein-DNA complex, protein-membrane complex and/or lipoprotein, is capable of specifically binding the immunogenic composition. If the antibody or functional fragment or functional equivalent is capable of specifically binding the resulting immunogenic composition, it shows that the epitope is still available for an animal's immune system.

The epitope of the peptide, polypeptide, protein, glycoprotein, protein-DNA complex, protein-membrane complex and/or lipoprotein is preferably surface-exposed when the peptide, polypeptide, protein, glycoprotein, protein-DNA complex, protein-membrane complex and/or lipoprotein is in its native conformation so that, after administration to a suitable host, an immune response against the native form of the peptide, polypeptide, protein, glycoprotein, protein-DNA complex, protein-membrane complex and/or lipoprotein is elicited.

A functional fragment of an antibody is defined as a fragment which has at least one same property as the antibody in kind, not necessarily in amount. The functional fragment is preferably capable of binding the same antigen as the antibody, albeit not necessarily to the same extent. A functional fragment of an antibody preferably comprises a single domain antibody, a single chain antibody, a Fab fragment or a F(ab′)2 fragment. A functional equivalent of an antibody is defined as a compound which is capable of specifically binding the same antigen as the antibody. A functional equivalent for instance comprises an antibody which has been altered such that the antigen-binding property of the resulting compound is essentially the same in kind, not necessarily in amount. A functional equivalent is provided in many ways, for instance through conservative amino acid substitution, whereby an amino acid residue is substituted by another residue with generally similar properties (size, hydrophobicity, etc), such that the overall functioning is likely not to be seriously affected.

The invention is further explained in the following examples. These examples do not limit the scope of the invention, but merely serve to clarify the invention.

### EXAMPLES

Abbreviations: AFM, atomic force microscopy; ANS, 1-anilino-8-naphthalene sulfonate; aPMSF, 4-Amidino-Phenyl)-Methane-Sulfonyl Fluoride; BCA, bicinchoninic acid; bis-ANS, 4,4′-dianilino-1,1′-binaphthyl-5,5′-disulfonic acid; CD, circular dichroism; CR, Congo red; CSFV, Classical Swine Fever Virus; DLS, dynamic light scattering; DNA, Deoxyribonucleic acid; dOVA, misfolded ovalbumin comprising cross-beta; ELISA, enzyme linked immuno sorbent assay; ESI-MS, electron spray ionization mass spectrometry; FPLC, fast protein liquid chromatography; g6p, glucose-6-phosphate; GAHAP, alkaline-phosphatase labeled goat anti-human immunoglobulin antibody; h, hour(s); H#, hemagglutinin protein of influenza virus, number #; HBS, HEPES buffered saline; HCV, hepatitis C virus; HGFA, Hepatocyte growth factor activator; HK, Hong kong; HPLC, high performance, or high-pressure liquid chromatography; HRP, horseradish peroxidase; hrs, hours; Ig, immunoglobulin; IgG, immunoglobulin of the class 'G; IgIV, immunoglobulins intravenous; kDa, kilo Dalton; LAL, Limulus Amoebocyte Lysate; MDa, mega Dalton; NMR, nuclear magnetic resonance; OVA, ovalbumin; PBS, phosphate buffered saline; Plg, plasminogen; RAGE, receptor for advanced glycation end-products; RAMPO, peroxidase labeled rabbit anti-mouse immunoglobulins antibody; RNA, ribonucleic acid; RSV, respiratory syncytial virus; RT, room temperature; SDS-PAGE, sodium-dodecyl sulphate-polyacryl amide gel electrophoresis; SEC, size exclusion chromatography; SWARPO, peroxidase labeled swine anti-rabbit immunoglobulins antibody; TEM, transmission electron microscopy; ThS, Thioflavin S; ThT, Thioflavin T; tPA, tissue type plasminogen activator; VN, Vietnam; W, tryptophan.

Activation of T-cells. Analysis of (primary) T cell responses by immunogenic compositions comprising amino-acid sequences with cross-beta conformation.

Isolation and culture of T cell populations. The ability of immunogenic compositions comprising amino-acid sequences with cross-beta conformation, referred to as “cross-beta-antigens,” to induce (primary) T cell responses in vivo is preferably tested in vitro using T cells isolated from immunized animals, for example mammals. For example, T cells are isolated from mice or from a human individual. Alternatively, activation of naïve T cells is analyzed upon isolation of T-cells from non-immunized animals, for example mammals, for example from mice or human individuals.

Several methods for T-cell isolation are known and commonly used in practice by persons skilled in the art. Preferably, T cells are isolated from blood or splenocytes, for example from splenocytes isolated from immunized mammals, for example mice. Mammals, for example mice are immunized with antigen, preferably immunogenic compositions comprising cross-beta adjuvant and peptide, polypeptide, protein, glycoprotein, protein-DNA complex, protein-membrane complex and/or lipoprotein comprising at least one T-cell epitope motif, preferably once or twice, and cells are isolated preferably between 3 and 14 days after immunization. Preferably, spleen cell suspensions or peripheral blood mononuclear cells are used. Splenocytes are preferably isolated using cell strainers, preferably with a pore size of 100 μm. Preferably, erythrocytes are removed from the cell suspension, preferably by a centrifugation step using Ficoll, or by hemolysis, preferably with a hypotonic buffer, preferably composed of ammonium chloride, preferably at 0.15 mM, and potassium bicarbonate, preferably at 0.1 mM, and ethylendiaminetetaacetic acid, preferably at 0.01 mM.

Subsequently, isolated and washed T-cells are used either directly for analysis of their response towards immunogenic compositions comprising cross-beta adjuvant and peptide, polypeptide, protein, glycoprotein, protein-DNA complex, protein-membrane complex and/or lipoprotein comprising at least one T-cell epitope motif or the isolated and washed T-cells are cultured in appropriate cell culture medium, preferably Dulbecco's Modified Eagle's Medium (DMEM) or RPMI, supplemented with 10% fetal calf serum or human serum, L-glutamine, penicillin, streptomycin and β-mercapto-ethanol, and in appropriate cell culture flasks, for example 96-wells or 24-wells culture systems at appropriate cell density, preferably approximately 5 to 35×10⁶ cells per ml. For example, such analyses are performed in an indirect way with antigen presenting cells included in the analyzed cell cultures, and/or directly by assessing responsiveness towards T-cell epitope motifs, for example using peptides of such motifs.

Analysis of T cell response. The number of antigen specific T cells is preferably measured directly, preferably using staining with pre-labeled tetrameric or pentameric MHC molecules, loaded with peptides derived from the antigen, i.e., T-cell epitope motifs, using a FACS apparatus. Preferably, between 5×10⁵ and 5×10⁶ cells are measured. In addition, the following T cell responses are preferably measured: cytokine production, T cell proliferation and cytotoxic activity of CD8⁺ T cells. For analysis of cytokines isolated cells are preferably cultured for 16 to 48 hrs in the presence of antigen, for example as an immunogenic compositions comprising cross-beta adjuvant and peptide, polypeptide, protein, glycoprotein, protein-DNA complex, protein-membrane complex and/or lipoprotein comprising at least one T-cell epitope motif, when antigen presenting cells are included in the analyzed cell cultures, or in the presence of T-cell epitope motifs, when cultures of T-cells only are assessed. Preferably a concentration series of immunogenic composition comprising cross-beta and T-cell epitope motif(s), and/or (a) peptide(s) with (an) amino acid sequence(s) of (a) T-cell epitope motif(s) is tested, preferably at concentrations between 10 ng to 500 μg/ml. For example, such cross-beta antigen is provided in the presence of heat shock proteins, such as hsp90, and/or in the presence of a selection of human antibodies, preferably a collection of IVIg, preferably a collection of IVIg selected by a method to enrich for antibodies directed towards cross-beta comprising molecules. Induction of cytokine production is preferably measured using a capture method, i.e., using bi-specific antibodies that bind to a common surface molecule on T-cells and to the cytokine to be analyzed on a FACS apparatus. Preferably interferon-γ (IFN-γ), IL-4 and IL-5 are measured and preferably T-cells are co-stained with antibodies for CD4⁺ and CD8⁺, respectively in order to distinguish the phenotype of the responding T cells. Alternatively, cytokine production is for example measured using ELISPOT analysis or ELISA. T cell proliferation is measured for example using ³H-Thymidine incorporation. Preferably proliferation is analyzed after 5-6 days of culture in the presence of antigen, for example provided as immunogenic compositions comprising cross-beta adjuvant and peptide, polypeptide, protein, glycoprotein, protein-DNA complex, protein-membrane complex and/or lipoprotein comprising at least one T-cell epitope motif, when antigen presenting cells are included in the analyzed cell cultures, or in the presence of T-cell epitope motifs, when cultures of T-cells only are assessed, referred to jointly as “antigen” for the two combined possibilities. Preferably a concentration series of such antigen is tested, preferably at concentrations between 10 ng to 500 μg/ml. Preferably the cells are pulsed with, preferably 0.5 μCi/50 μl ³H-Thymidine for the final 6 to 24 hours. Alternatively, proliferation is measured using BrdU or CSFE. For measurement of cytotoxic activity splenocytes isolated from syngeneic animals are for example used as target cells. Target cells are preferably prepared using antigen, for example immunogenic compositions comprising cross-beta adjuvant and peptide, polypeptide, protein, glycoprotein, protein-DNA complex, protein-membrane complex and/or lipoprotein comprising at least one T-cell epitope motif, when antigen presenting cells are included in the analyzed cell cultures, or using peptides of T-cell epitope motifs, for 16-48 hr or 1-4 hours, respectively, and loaded with ⁵¹Cr. Preferably a concentration series of such antigen is tested, preferably at concentrations between 10 ng to 500 μg/ml. After removal of free ⁵¹Cr by washing preferably around 3000 cells are used in a 96 well cluster. Lysis of target cells is measured by the release ⁵¹Cr of following the addition of responder cells, derived from the splenocytes stimulated with antigen, for example immunogenic compositions comprising cross-beta adjuvant and peptide, polypeptide, protein, glycoprotein, protein-DNA complex, protein-membrane complex and/or lipoprotein comprising at least one T-cell epitope motif, or with peptides of T-cell epitope motifs. Preferably a titration of responder cells is tested in ratios of preferably 1:1 to 1:40 with target cells. Alternatively, other target cells, such as tumor cells are for example used, for example E.G7-OVA cells or tumor cells, such as B lymphomas that can be triggered to present peptides.

For example, mice are immunized with an immunogenic composition comprising ovalbumin as the cross-beta-adjuvated antigen comprising T-cell epitope motifs. Alternatively, human FVIII, E2 derived from classical swine fever virus (CSFV), H5 from influenza virus H5N1 strain A/VN/1203/04 or strain A/HK/156/97, or another protein is used in immunogenic compositions comprising cross-beta adjuvant and T-cell epitope motifs, for example. A cross-beta adjuvant protein is the source of T-cell epitope motifs, and/or a cross-beta adjuvant protein is coupled to an antigen and/or coupled to (a) peptide(s). Preferably, the coupled antigen and/or the coupled peptide(s) are known to be able to generate a T cell response, and/or are predicted to be able to generate a T cell response, preferably by using algorithms and computer based analysis, for example using software such as BIMAS, SYFPEITHI or RANKPEP. For example, such peptides are derived from pathogens, for example from the proteins of influenza virus, for example from H5N1, for example from the nucleoprotein or for example from proteins of human immunodeficiency virus (HIV), plasmodium falciparum, mycobacterium tuberculosis. Such examples include, but are by no means restricted to, peptide AMQMLKETI of the gag24 protein of HIV, and peptides IYSTVASSL, LYQNPTTYI, TYISVGTST, KYVKSNRLV, DYEELKHLL, SYNNTNQEDL, TYISVGTSTL, and KYVKSNRLVL of influenza virus, and in general any known or predicted peptide is used and mixed or coupled with the cross-beta-adjuvated protein. Alternatively, such peptides spanning T-cell epitope motifs are derived from antigens known or predicted to be targets in immunotherapy for cancer or other (human) disease, such as atherosclerosis.

Alternative to primed T cells isolated from immunized non-human animals, or humans which had previously been exposed to an antigen of interest, T cells derived from transgenic animals or T cell clones are for example used. For example, OT-I, OT-II, RF33 or D011.10 cells are used, T cells that are specific for peptides derived from ovalbumin presented in the context of specific MHC class I or MHC class II molecules, respectively peptide SIINFEKL (amino acid residues 257-264) and MHC class I allele Kb for RF33, peptide VAAHAEINEA (327-337) and MHC class II allele IAd for D011.10, peptide SIINFEKL (amino acid residues 257-264) and MHC class I allele Kb for OT-I, peptide AAHAEINEAG (328-338) and MHCII allele IAb for OT-II. Alternatively, one of the T cell hybridomas B3Z, B)97.10 or 54.8 is for example used. Alternative to splenocytes or monocytes as source of antigen presenting cells, cell lines are for example used as antigen presenting cells, such as for example D1 or DC2.4.

Alternative to in vivo primed T cells, naive T cells are for example used in cultures comprising antigen presenting cells and/or in cultures with T-cell only, to analyze the ability of immunogenic compositions comprising cross-beta-adjuvated antigen and T-cell epitope motifs, or of peptides spanning T-cell epitope motifs, to activate the T-cells, respectively. Since the number of T cells specific for the peptides spanning T-cell epitope motifs is low the isolated cells are preferably cultured in the presence of mature antigen presenting cells and immunogenic compositions comprising cross-beta-adjuvated antigen and T-cell epitope motifs for preferably around 1 week and subsequently for a prolonged period, preferably several weeks and preferably in the presence of several cytokines, preferably IL-2, PGE2, TNFα and IL-6 to induce optimal expansion of antigen specific T cells. After expansion, T cells are triggered with peptides spanning T-cell epitope motifs for preferably 1 to 6 days and analyzed, preferably as described above for primed T cells, for the production of cytokines and/or for their ability to proliferate in response to specific peptides spanning T-cell epitope motifs.

Analysis of efficacy of immunogenic compositions comprising T-cell epitope motifs and cross-beta adjuvant in vivo. Immunizations using immunogenic compositions comprising T-cell epitope motifs and cross-beta adjuvant are preferably aimed at inducing protection against a challenge with a pathogen, and/or aimed at treating a disease. Preferably, the capacity of cross-beta adjuvant protein to induce an effective immune response is analyzed in vivo. For example, non-human animals are immunized with immunogenic compositions comprising T-cell epitope motifs and cross-beta adjuvant to induce protection against a challenge with a pathogen, for example a virus, bacteria or parasite. For example, non-human mammals are immunized with immunogenic compositions comprising T-cell epitope motifs and cross-beta adjuvant, comprising for example H5 and/or peptides thereof, and are subsequently challenged with influenza virus. For example, such challenge is with strain A/HK/156/97 or A/VN/1203/04. In another example, pigs are immunized with immunogenic compositions comprising T-cell epitope motifs and cross-beta adjuvant, comprising E2 protein and/or peptides thereof, and or another protein derived from the sequences of the genes encoding proteins of Classical Swine Fever Virus, and challenged with Classical Swine Fever Virus, for example of strain Brescia 456610. Effectiveness of immunization with immunogenic compositions comprising T-cell epitope motifs and cross-beta adjuvant, for the treatment of a disease, for example cancer, when for example a tumor antigen is incorporated in the immunogenic composition, or for example atherosclerosis, is preferably analyzed in immunized mammals. For example an effective immune response is determined by performing an in vivo tumor experiment. For example this is performed using an immunogenic composition comprising ovalbumin as the cross-beta-adjuvated antigen comprising T-cell epitope motifs as antigen and ovalbumin expressing tumor cells, for example E.G7 cells. After immunization with the immunogenic composition as described, after preferably 7 days, animals are injected intradermally in the back with 5×10⁵ E.G7 tumor cells, which were washed preferably in PBS before injection, preferably in a volume of 200 μl. The mice are then examined in time to monitor tumor growth. The tumor growth is preferably estimated by determining the largest and smallest diameters of the tumors and calculating their size. In another example, the mammals are immunized with immunogenic compositions comprising T-cell epitope motifs and cross-beta adjuvant with proteins comprising amino-acid sequences of human papilomavirus proteins (HPV), preferably from the E6 or E7 protein, and challenged with HPV. In another example, the mammals, preferably mammals suffering from atherosclerosis, preferably mice or human, are immunized with immunogenic compositions comprising T-cell epitope motifs and cross-beta adjuvant, for example oxidized LDL and/or glycated protein, for example glycated albumin, and analyzed for progression of diseases, preferably by measuring the size of the atherosclerotic plaque, by determining cytokine levels and/or by scoring survival rates.

A surrogate marker for T-cell activation in mice in vivo: determination of IgG1/IgG2a titer ratio. As a surrogate marker for the occurrence of a T-cell activation in vivo upon subjecting an animal, for example a mouse, to immunizations with an immunogenic composition comprising cross-beta and T-cell epitope motifs, titers of IgG1 and IgG2a are preferably determined using an ELISA with immobilized antigen and dilution series of immune serum, according to methods and protocols known to a person skilled in the art. Increase in IgG1 titers, when compared to pre-immune serum and/or serum of the animal(s) that received placebo, is an indicative measure for the occurrence of a T-helper 2 mediated humoral response, with activation of CD4+ T-helper cells. Increase in IgG2a titers, when compared to pre-immune serum and/or serum of the animal(s) that received placebo, is an indicative measure for the occurrence of a T-helper 1 mediated cellular immune response, with activation of CD8+ cytotoxic T-cells. In addition, total IgG titers are determined as a indicative measure for activation of CD4+ positive T-helper cells.

T-cell activation: summary. Disappearing Epitope Scanning Technology of this Example comprises two main approaches resulting in the ability of selecting from a plurality of immunogenic compositions those immunogenic compositions having a greater chance of being capable of eliciting and/or stimulating a protective prophylactic immune response and/or a therapeutic immune response in vivo, as compared to the other immunogenic compositions of a plurality of immunogenic compositions. The elicited immune response comprises activation of T-cells, for example resulting in a CD4+ T-help response, and/or resulting in a CD8+ cytotoxic T-lymphocyte response. When T-cell epitope motifs are not known for an antigen and/or when T-cell epitope motifs are not adequately or not at all predicted by algorithms and computer based analysis, approach I is preferred:

Approach I. Design of Immunogenic Compositions Comprising One or More T-Cell Epitope Motifs and Cross-Beta Adjuvant, Checked for Functionality with Cell Cultures of APCs+Naïve and/or Primed T-Cells.

When applying approach I. of the Disappearing Epitope Scanning Technology, one predicted and/or putative T-cell epitope motif and/or series of predicted and/or putative motifs are incorporated in immunogenic compositions comprising cross-beta adjuvant. Putative T-cell epitope motifs are for example obtained by synthesizing peptides covering overlapping sequences of the antigen, comprising preferably the number of amino-acid residues known to be required for presentation by major histocompatibility complexes, for example 5-30 amino-acid residues. The sequence overlap between two adjacent peptides is for example 1-10 amino-acid residues.

When T-cell epitope motifs are known and/or when algorithms and computer based analysis predict T-cell epitope motifs accurately to a large extent, approach II of the Disappearing Epitope Scanning Technology is preferred:

Approach II. Design of Ready-to-Use Immunogenic Compositions Comprising One or More Known and/or Predicted T-Cell Epitope Motifs and Cross-Beta Adjuvant.

Peptides spanning T-cell epitope motifs are (1) predicted T-cell epitope motifs (MHC class I restricted or MHC class II restricted) obtained using prediction programs, and/or are (2) known T-cell epitope motifs, like for example, but not limited to, those identified for H5 or OVA. The known and/or predicted T-cell epitope motifs are (i) part of the cross-beta-adjuvated antigen comprising the motifs, and/or are (ii) part of a natively folded antigen comprising the motifs, that is (a) coupled and/or mixed with cross-beta-adjuvated antigen comprising the motifs, and/or that is (b) coupled and/or mixed with cross-beta-adjuvated protein with unrelated amino-acid sequence with respect to the amino-acid sequence of the parent antigen from which the peptides are derived, for use as an immunogenic composition in vivo, as a vaccine candidate preceding a challenge with tumor cells or pathogen, and/or with the purpose to obtain primed T-cells, and/or for use as an immunogenic composition in vitro for assessing T-cell activation in vitro, by using co-cultures of APCs and naïve and/or primed T-cells, and/or T-cell clones specific for a known T-cell epitope motif, and/or (iii) used as sole peptides (a) having conformations covering those folds that are present when the peptides are presented by major histocompatibility complexes at APCs, for assessing direct stimulation of cultured naïve and/or primed T-cells, and/or T-cell clones specific for a known T-cell epitope motif, in the presence of the selected major histocompatibility complexes, or (b) comprising cross-beta conformation for 4-75%, and/or (c) coupled to and/or mixed with cross-beta-adjuvated antigen comprising the motifs, and/or (d) coupled to and/or mixed with cross-beta-adjuvated protein with unrelated amino-acid sequence with respect to the amino-acid sequence of the parent antigen from which the peptides are derived, for assessing T-cell activation in vivo upon immunization, and/or for obtaining primed T-cells upon immunizations, and/or for assessing T-cell activation in vitro, by using co-cultures of APCs and naïve and/or primed T-cells and/or T-cell clones specific for a known T-cell epitope motif.

Animal or human individuals that have T-cell clones specific for T-cell epitope motifs under investigation, upon previous immunization with an antigen comprising T-cell epitope motifs, for example upon vaccination and/or for example upon suffering and subsequent recovering from an infection, are serving as a source of T-cells used for the aforementioned experiments comprising cultured primed T-cells.

### Detection of Proteins Comprising Cross-Beta

Protein misfolding and cross-beta structure. Several techniques are generally available by a person skilled in the art to analyze the presence of cross-beta, i.e., non-native structural elements in unfolded proteins, misfolded proteins and multimerized forms thereof. For example, and as described in more detail below, these techniques allow the detection of non-native epitopes, the detection of the size of the misfolded proteins and multimers thereof and the analysis of the shape of the aggregates. Combined, these techniques allow detailed description of the presence and characteristics of proteins comprising cross-beta. Therefore these techniques allow the description of immunogenic compositions comprising cross-beta. Preferably, when applying any of the techniques described below, a reference sample of the non-treated protein is compared to the protein that is subjected to misfolding procedures, for comparison.

### Cross-Beta Detection Assays

Congo red fluorescence. Congo red is a relatively small molecule (chemical name: C₃₂H₂₂N₆Na₂O₆S₂) that is commonly used as histological dye for detection of amyloid. The specificity of this staining results from Congo red's affinity for binding to fibrillar proteins enriched in beta-sheet conformation and comprising cross-beta. Congo red is also used to selectively stain protein aggregates with amyloid properties that do not necessarily form fibrils. Congo red is also used in a fluorescence enhancement assay to identify proteins with cross-beta in solution. This assay, also termed Congo red fluorescence measurement, is for example performed as described in patent application WO2007008072, the contents of the entirety of which are incorporated herein by this reference, paragraph [101]. Fluorescence can be read on various readers, for example fluorescence is read on a Gemini XPS microplate reader (Molecular Devices).

Thioflavin T fluorescence. Thioflavin T, like Congo red, is also used by pathologists to visualize plaques composed of amyloid. It also binds to beta sheets, such as those in amyloid oligomers. The dye undergoes a characteristic 115 nm red shift of its excitation spectrum that may be selectively excited at 442 nm, resulting in a fluorescence signal at 482 nm. This red shift is selectively observed if structures of amyloid fibrillar nature are present. It will not undergo this red shift upon binding to precursor monomers or small oligomers, or if there is a high beta sheet content in a non-amyloid context. If no amyloid fibrils are present in solution, excitation and emission occur at 342 and 430 nm respectively. Thioflavin T is often used to detect cross-beta in solutions. For example, the Thioflavin T fluorescence enhancement assay, also termed “ThT fluorescence measurement”, is performed as described in patent application WO2007008072, paragraph [101]. Fluorescence can de read on various readers, for example fluorescence is read on a Gemini XPS microplate reader (Molecular Devices).

Thioflavin S fluorescence. Thioflavin S, is a dye similar to Thioflavin T and the fluorescence assay is performed essentially similar to ThT and CR fluorescence measurements.

tPA binding ELISA. tPA binding ELISA with immobilized misfolded proteins; is performed as described in patent application WO2007008070, paragraph [35-36]. One of our first discoveries was that tPA binds specifically to misfolded proteins comprising cross-beta. Binding of tPA to misfolded proteins is mediated by its finger domain. Other finger domains and proteins comprising homologous finger domains are also applicable in a similar ELISA setup (see below).

BiP binding ELISA. BiP binding ELISA with immobilized misfolded proteins; is performed as described in patent application WO2007108675, section “Binding of BiP to misfolded proteins with cross-beta structure”, the contents of the entirety of which are incorporated herein by this reference, with the modification that BiP purified from cell culture medium using Ni²⁺ based affinity chromatography, is used in the ELISAs. It has been demonstrated previously that chaperones like for example BiP bind specifically to misfolded proteins comprising cross-beta. Other heat shock proteins, such as hsp70, hsp90 are also applicable in a similar ELISA setup.

IgIV binding ELISA. Immunoglobulins intravenous (IgIV) binding ELISA with immobilized misfolded proteins; is performed as described in patent application WO2007094668, the contents of the entirety of which are incorporated herein by this reference, paragraph [0115-0117]. Alternatively, IgIV that is enriched using an affinity matrix with immobilized protein(s) comprising cross-beta, is used for the binding ELISA with immobilized misfolded proteins (see patent application WO2007094668, paragraph [0143]). It has been demonstrated previously that a subset of immunoglobulins in IgIV bind selectively and specifically to misfolded proteins comprising cross-beta. Other antibodies directed against misfolded proteins are also applicable in a similar ELISA setup.

Finger binding ELISA using fibronectin finger domains. Fibronectin finger 4-5 binding ELISA with immobilized misfolded proteins; is performed as described in patent application WO2007008072. It has been demonstrated previously that finger domains of fibronectin selectively and specifically bind to misfolded proteins comprising cross-beta. In addition to, or alternative to finger domains of fibronectin, finger domains of tPA and/or factor XII and/or hepatocyte growth factor activator are used.

Factor XII activation assay. Factor XII/prekallikrein activation assay is performed as described in patent application WO2007008070, paragraph [31-34]. It has been demonstrated previously that factor XII selectively and specifically bind to misfolded proteins comprising cross-beta, resulting in its activation.

tPA/plasminogen activation assay. Enhancement of tPA/plasminogen activity upon exposure of the two serine proteases to misfolded proteins was determined using a standardized chromogenic assay (see for example patent application WO2006101387, the contents of the entirety of which are incorporated herein by this reference, paragraph [0195], patent application WO2007008070, paragraph [31-34], and [Kranenburg et al., 2002, Curr. Biology 12(22), pp. 1833)]. Both tPA and plasminogen act in the Cross-beta Pathway. Enhancement of the activity of the cross-beta binding proteases is a measure for the presence of misfolded proteins comprising cross-beta structure. 4-Amidinophenylmethanesulfonyl fluoride hydrochloride (aPMSF, Sigma, A6664) was added to protein solutions to a final concentration of 1.25 mM from a 5 mM stock. Protein solutions with added aPMSF were kept at 4° C. for 16 h before use in a tPA/plasminogen activation assay. In this way, proteases that are putatively present in protein solutions to be analyzed, and that may act on tPA, plasminogen, plasmin and/or the chromogenic substrate for plasmin, are inactivated, to prevent interference in the assay.

Binding assays. Apart from the above described binding assays using cross-beta binding compounds, additional cross-beta binding compounds are suitable for use in binding assays for determination of the presence and extent of cross-beta in a sample of a peptide, polypeptide, protein, glycoprotein, protein-DNA complex, protein-membrane complex and/or lipoprotein. In general, cross-beta binding compounds useful for these determinations are tPA, BiP, factor XII, fibronectin, hepatocyte growth factor activator, at least one finger domain of tPA, at least one finger domain of factor XII, at least one finger domain of fibronectin, at least one finger domain of hepatocyte growth factor activator, Thioflavin T, Thioflavin S, Congo Red, CD14, a multiligand receptor such as RAGE or CD36 or CD40 or LOX-1 or TLR2 or TLR4, a cross-beta-specific antibody, preferably cross-beta-specific IgG and/or cross-beta-specific IgM, IgIV, an enriched fraction of IgIV capable of specifically binding a cross-beta structure, Low density lipoprotein Related Protein (LRP), LRP Cluster II, LRP Cluster IV, Scavenger Receptor B-I (SR-BI), SR-A, chrysamine G, a chaperone, a heat shock protein, HSP70, HSP60, HSP90, gp95, calreticulin, a chaperonin, a chaperokine and/or a stress protein. In addition, as disclosed previously in patent application WO2007008072, cross-beta binding compounds for use for the aforementioned determinations are 2-(4′-(methylamino)phenyl)-6-methylbenzothiaziole, styryl dyes, BTA-1, Poly(thiophene acetic acid), conjugated polyeclectrolyte, PTAA-Li, Dehydro-glaucine, Ammophedrine, isoboldine, Thaliporphine, thalicmidine, Haematein, ellagic acid, Ammophedrine HBr, corynanthine, Orcein.

### Measurements of Protein Refolding and Changes in Protein Conformation & Multimer Size and Multimer Size Distribution Analysis

Turbidity of protein solutions. With turbidity measurements the diffraction of light scattered by protein particles in the sample is detected. Light is scattered by the solid particles and absorbed by dissolved protein. In a turbidity measurement the amount of insoluble particles in a solution is determined. This aspect is used to determine the amount of insoluble protein in samples of protein that is subjected to misfolding conditions, compared to the fraction of insoluble protein in the non-treated reference sample.

Recording changes in binding characteristics of binding partners for a protein. Antibodies specific for a protein in a certain conformation are used to measure the amount of this protein present in this specific state. Upon treatment of the protein using misfolding conditions, binding of antibodies is inhibited or diminished, which is used as a measure for the progress and extent of misfolding. In addition or alternatively, antibodies are used that are specific for certain conformations and/or post-translational modifications, for example glycation, oxidation, citrullination (gain of binding to the protein subjected to misfolding conditions). When for example glycation and/or oxidation and/or citrullination procedures is/are part of the misfolding procedure, the effect of the treatment with respect to the occurrence of modified amino-acid residues is recorded by determining the relative binding of the antibodies, compared to the non-treated reference protein. Alternatively or in addition to the use of antibodies, any binding partner and/or ligand of the non-treated protein is used similarly, and/or any binding partner and/or ligand other than antibodies, of the misfolded protein is used. When a protein changes conformation ligands or binding partners express altered binding characteristics, which is used as a measure for the extent of protein modification and/or extent of misfolding. This binding of antibodies, ligands and/or binding partners is measured using various techniques, such as direct and/or indirect ELISA, surface plasmon resonance, affinity chromatography and immuno-precipitation approaches.

Differential scanning calorimetry/micro DSC for detecting changes in protein conformation. Differential scanning calorimetry (DSC) is a thermo-analytical technique in which the difference in the amount of heat required to increase the temperature of a sample and a reference is measured as a function of temperature. The temperature is linearly increased over time. When the protein in the sample changes its conformation, more or less heat (depending on if it is an endo- or exothermic reaction) will be required to increase the temperature at the same rate as the reference sample. In this way the conformational changes as a result of an increase in temperature can be measured.

Particle analyzer. A particle analyzer measures the diffraction of a laser beam when targeted at a sample. The resulting data is transformed by a Fourier transformation and gives information about particle size and shape. When applied to protein solutions, putatively present protein aggregates are detected, when larger than the lower detection limit of the apparatus, for example in the sub-micron range.

Direct light microscope. With a regular direct-light microscope with a preferable magnification range of 10×-100×, one can determine visually if there are any protein aggregates present in a sample.

Photon correlation spectroscopy (dynamic light scattering spectroscopy). Photon correlation spectroscopy can be used to measure particle size distribution in a sample in the nm-μm range.

Nuclear magnetic resonance spectroscopy. Nuclear Magnetic Resonance Spectroscopy (NMR) can be used to assess the electromagnetic properties of certain nuclei in proteins. With this technique the resonance frequency and energy absorption of protons in a molecule are measured. From this data structural information about the protein, like angles of certain chemical bonds, the lengths of these bonds and which parts of the protein are internally buried, can be obtained. This information can then be used to calculate the complete three dimensional structure of a protein. This method however is normally restricted to relatively small molecules. However with special techniques like incorporation of specific isotopes and transverse relaxation optimized spectroscopy, much larger proteins can now be studied with NMR.

X-ray diffraction. In X-ray diffraction with protein crystals, the elastic scattering of X-rays from a crystallized protein is measured. In this way the arrangement of the atoms in the protein can be determined, resulting in a three-dimensional structural model of the protein. First a protein is crystallized and then a diffraction pattern is measured by irradiating the crystallized protein with an X-ray beam. This diffraction pattern is a representation of how the X-ray beam is scattered from the electrons in the crystal. By gradually rotating the crystal in the X-ray beam, the different atomic positions in the crystal can be determined. This results in an electron density map, with which a complete three-dimensional atomic model of the crystallized protein can be calculated, regularly at the 1-3 Å scale. In this model it can be deduced whether protein molecules underwent conformational changes upon treatment with misfolding conditions, when compared to the structural model of the non-treated protein. In addition, modifications of amino-acid residues become apparent in the structural model, as well as whether the protein molecule forms ordered multimers of a defined size, like for example in the range of dimers-octamers.

Determination of the presence of cross-beta in fibers comprising crystallites, and/or in other appearances of protein aggregates comprising at least a fraction of the protein molecules in a crystalline ordering, can be assessed using X-ray fiber diffraction, as for example shown in [Bouma et al., J. Biol. Chem. V278, No. 43, pp. 41810-41819, 2003, “Glycation Induces Formation of Amyloid Cross-beta Structure in Albumin”].

Fourier Transform infrared spectroscopy. Detection of protein secondary structure in Fourier Transform Infrared Spectroscopy (FTIR), an infrared beam is split in two separate beams. One beam is reflected on a fixed mirror, the second on a moving mirror. These two beams together generate an interferogram which consists of every infrared frequency in the spectrum. When transmitted through a sample, specific functional groups in the protein adsorb infrared of a specific wavelength. The resulting interferogram must be Fourier transformed, before it can be interpreted. This Fourier transformed interferogram gives a plot of al the different frequencies plotted against their adsorption. This interferogram is specific for the structure of a protein, like a “molecular fingerprint,” and provides information on types of atomic bonds present in the molecule, as well as the spatial arrangement of atoms in for example alpha-helices or beta-sheets.

8-Anilino-1-naphthalenesulfonic acid fluorescence enhancement assay. 8-Anilino-1-naphthalenesulfonic acid (ANS) fluorescence enhancement assay, or ANS fluorescence measurement; was performed as described in patent application WO2007094668, the contents of the entirety of which are incorporated herein by this reference. Modification: fluorescence is read on a Gemini XPS microplate reader (Molecular Devices).

ANS is a chemical binds to hydrophobic surfaces of a protein and its fluorescence spectrum shifts upon binding. When proteins are in an unfolded state, they generally display more hydrophobic sites, resulting in an increased ANS shift compared to the protein in its native more globular state. ANS can therefore be used to measure protein unfolding.

bis-ANS fluorescence enhancement assay. 4,4′ dianilio-1,1′ binaphthyl-5,5′ disulfonic acid di-potassium salt (Bis-ANS) fluorescence enhancement assay; is performed as described in patent application WO2007094668. Essentially, bis-ANS has characteristics comparable to ANS, and bis-ANS is also used to probe for differences in solvent exposure of hydrophobic patches of proteins, when measuring bis-ANS binding with a reference protein samples, and with a protein sample subjected to a misfolding procedure.

Gel electrophoresis. Gel electrophoresis using sodium dodecyl-sulphate polyacryl amide gels (SDS-PAGE) and Coomassie stain, with various gels with resolutions between for example 100 Da up to several thousands of kDa, provides information on the occurrence of protein modifications and on the occurrence of multimers. Multimers that are not covalently coupled may also appear as monomers upon the assay conditions applied, i.e., heating protein samples in assay buffer comprising SDS. Samples are heated in the presence or absence of a reducing agent like for example dithiothreitol (DTT), when the protein amino-acid sequence comprises cysteines, that can form disulphide bonds upon subjecting the protein to misfolding conditions.

Western blot. When antibodies are available that bind to epitopes on the protein under the denaturing conditions as applied during SDS-PAGE, Western blotting is performed with the same protein samples as applied for SDS-PAGE with Coomassie stain, using the same molecular weight cut-off gels, and using the same protein sample handling approaches.

Centrifugation. Centrifugation and subsequent comparing the protein concentration in the supernatant with respect to the concentration before centrifugation provides insight into the presence of insoluble precipitates in a protein sample. Upon applying increasing g-forces for a constant time, and/or upon applying fixed or increasing g-forces for an increasing time frame, to a protein solution, with analyzing the protein content in between each step, information is gathered about the presence of insoluble multimers. For example, protein solutions are subjected for 10 minutes to 16,000*g, or for 60 minutes to 100,000*g. The first approach is commonly used to prepare protein solutions for, for example use on FPLC columns or in biological assays, with the aim of pelleting insoluble protein aggregates and using the supernatant with soluble protein. It is generally accepted that after applying 100,000*g for 60 minutes to a protein solution, only soluble multimers are left in the supernatant. As multimers ranging from monomers up to huge multimers comprising thousands of protein monomers may all have a density equal to the density of the buffer solution, applying these g-forces to protein solutions does not separate exclusively on size, but on density differences between the solution and the protein multimers.

Electron spray ionization mass spectrometry. Electron spray ionization mass spectrometry (ESI-MS) with protein solutions provides information on the multimer size distribution when sizes range from tens of Da up to the MDa range.

Ultrasonic spectrometry. Ultrasonic spectroscopy analysis, for example using an Ichos-II (Process Analysis and Automation, Ltd), provides insight into protein conformation and changes in tertiary structure are measured. In addition the technique can provide information on particle size of protein assemblies, and allows for monitoring protein concentration.

Dialysis (membranes with increasing molecular weight cut-off). Using one or a series of dialysis membranes with varying molecular weight cut-offs, size distribution/multimer distribution of protein can be assessed at the sub-oligomer scale, depending on the molecular weight of the monomer. Protein concentration analysis between each dialysis step with gradually increasing pore size (suitable for molecular weight ranges between approximately 1000-50000 Da). Protein concentration is for example monitored using BCA or Coomassie+ determinations (Pierce), and/or absorbance measurements at 280 nm, using for example the nanodrop technology (Attana).

Filtration (filters with increasing molecular weight cut-off). Filtration using a series of filters with gradually increasing MW cut-offs, ranging from the monomer size of the protein under investigation up to the largest MW cut-off available, reveals information on the distribution and presence of protein molecules in multimers in the range from monomers, lower-order multimers and large multimers comprising several hundreds of monomers. For example, filters with a MW cut-off of 1 kDa up to filters with a cut-off of 5 μm (MWs, for example, 1/3/10/30/50/100 kDa, completed with filters with cut-offs of for example 200/400/1000/5000 nm). In between each subsequent filtration step, protein concentration is assessed using for example the BCA or Coomassie+method (Pierce), and/or visualization on SDS-PA gel stained with Coomassie.

Transmission electron microscopy. Transmission electron microscopy (TEM) is an imaging technique that provides structural information of proteins at a nm to μm scale. With this resolution it is possible to identify the occurrence of protein assemblies ranging from monomers up to multimers of several thousands molecules, depending on the molecular weight of the parent protein molecule. Furthermore, TEM imaging provides insight into the structural appearance of protein multimers. For example, protein multimers appear as rods, globular structures, strings of globular structures, amorphous assemblies, unbranched fibers, commonly termed fibrils, branched fibrils, and/or combinations thereof.

In the current studies, TEM images were collected using a Jeol 1200 EX transmission electron microscope (Jeol Ltd., Tokyo, JP) at an excitation voltage of 80 kV. For each sample, the formvar and carbon-coated side of a 100-mesh copper or nickel grid was positioned on a 5 μl drop of protein solution for 5 min. Afterwards, it was positioned on a 100 μl drop of PBS for 2 minutes, followed by three 2-minute incubations with a 100 μl drop of distilled water. The grids were then stained for 2 minutes with a 100 μl drop of 2% (m/v) methylcellulose with 0.4% uranyl acetate pH 4. Excess fluid was removed by streaking the side of the grids over filter paper, and the grids were subsequently dried under a lamp. Samples were analyzed at a magnification of 10K.

Atomic force microscopy. Similar to TEM imaging, atomic force microscopy provides insights into the structural appearance of protein molecules at the protein monomer level up to the macroscopic level of large multimers of protein molecules.

Size exclusion chromatography, or gel filtration chromatography. With size exclusion chromatography (SEC) using HPLC and/or FPLC, a qualitative and quantitative insight is obtained about the distribution of protein molecules over monomers up to multimers, with a detectable size limit of the multimers restricted by the type of SEC column that is used. SEC columns are available with the ability to separate molecular sizes in the sub kDa range up to in the MDa range. The type of column is selected based on the molecular weight of the analyzed protein, and on any indicative information at forehand about the expected range of multimeric sizes. A reference non-treated protein may be compared to a protein subjected to misfolding procedures.

Tryptophan fluorescence. Assessment of differences in tryptophan (W) fluorescence intensity between two appearances of the same protein provides information on the occurrence of protein folding differences. In general, in globular proteins W residues are mostly buried in the interior of the globular fold. Upon unfolding, refolding, misfolding, W residues tend to become more solvent exposed, which is recorded in the W fluorescence measurement as a change in fluorescent intensity compared to the protein with a more native fold.

Dynamic Light Scattering. With the Dynamic Light Scattering (DLS) technique, particle size and particle size distribution are assessed. When protein solutions are considered, distribution of proteins over a range of multimers ranging from monomers up to multimers is measured, with the upper limit of detected multimer size limited by the resolution of the DLS technique.

Circular dichroism spectropolarimetry. With circular dichroism spectropolarimetry (CD) the relative presence of protein secondary structural elements is determined. Therefore, this technique allows for the comparison of the relative occurrence of alpha-helix, beta-sheet and random coil between a reference protein that is non-treated, and the protein that is subjected to misfolding conditions. An example of a CD experiment for assessment of conformational changes in proteins upon treatment with misfolding conditions is given in Bouma et al. “Glycation Induces Formation of Amyloid Cross-beta Structure in Albumin”, J. Biol. Chem. 278(43):.41810-41819 (2003).

Native gel electrophoresis. Distribution over multimers in the range of approximately monomers up to 100-mers is assessed by applying native gel electrophoresis. For this purpose a reference non-treated protein sample is compared to a protein sample which is subjected to a misfolding procedure. When misfolding procedures are applied that introduce modifications on amino-acid residues, like for example but not limited to, glycation or oxidation or citrullination, these changes are becoming apparent on native gels, as well.

Examples of Proteins that are Used for Preparation of Immunogenic Compositions

Envelope protein E2 of Classical Swine Fever Virus. The envelope protein E2 of Classical Swine Fever Virus (CSFV) strain Brescia 456610 is used as a prototype subunit vaccine candidate for examples described below. Currently, a subunit vaccine that provides protection in pigs against CSF comprises recombinantly produced E2 antigen in cell culture medium, adjuvated with a double emulsion of water-in-oil-in-water, comprising PBS, Marcol 52, Montanide 80. The vaccine comprises at least 32 μg E2/dose of 2 ml, and is injected intramuscularly.

E2 was recombinantly produced in insect Sf9 cells (Animal Sciences Group, Lelystad, NL) or in human embryonic kidney 293 cells (293) (ABC-Protein Expression facility, University of Utrecht, NL), as described in patent application WO2007008070. E2 produced in Sf9 cells and lacking any tags is in PBS after dialysis of cell culture medium (storage of aliquots at −20° C. or at −80° C.), or in cell culture medium (storage at −20° C.). Cell culture medium is SF900 II medium with 0.2% pluronic (serum free). After culturing of cells, the cell culture medium is micro-filtrated. Virus is inactivated with 8-12 mM 2-bromo-ethyl-ammonium bromide. The E2 produced in 293 cells comprises a C-terminal FLAG-tag followed by a His-tag, and is purified using Ni²⁺-based affinity chromatography. Concentration and purity of E2 from both sources is determined as follows. Quantification of the total protein concentration is performed with the BCA method (Pierce) or with the Coomassie+method (Pierce). E2 specific bands on a Western blot are visualized using anti-FLAG antibody (mouse antibody, M2, peroxidase conjugate; Sigma, A-8592) for the E2-FLAG-His construct, and a 1:1:1 mixture of three horseradish peroxidase (HRP) tagged mouse monoclonal anti-E2 antibodies (CediCon CSFV 21.2, 39.5 and 44.3; Prionics Lelystad) for the E2-FLAG-His construct and the E2 construct from Sf9 cells. The purity of E2 batches was determined by densitometry with a Coomassie stained sodium dodecyl sulphate-polyacryl amide (SDS-PA) gel after electrophoresis.

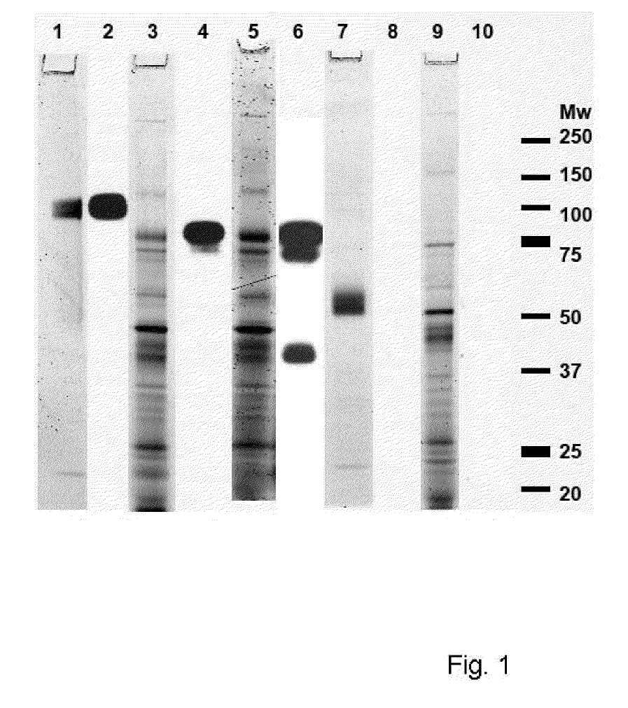

In FIG. 1, SDS-PA gels and Western blots with E2 produced in Sf9 cells and E2-FLAG-His produced in 293 cells are shown, with reducing and non-reducing conditions. It is clearly seen that the main fraction of both E2 batches appears as dimers on the gel and blot, when applied with non-reducing sample buffer. Apparently, those dimers are covalently coupled, since treatment of E2 from 293 cells with DTT reveals monomers at the expected molecular weight of approximately 47 kDa. No E2 bands are visualized on the blot when analyzing E2 from Sf9 cells under reducing conditions. The observation that E2 appears as at least two monomer and dimer bands is most likely related to the presence of glycosylation isoforms.

Before use in misfolding procedures, cross-beta analyses, multimer analyses and/or immunization, non-treated E2 solution was warmed to 37° C. for 10-30 minutes, left on a roller device for 10-30 minutes, at room temperature, warmed again at 37° C. for 0-30 minutes and left again on a roller device for 0-30 minutes. Alternatively, non-treated E2 solutions were quickly thawed at 37° C. and directly kept on wet ice until further use.

Ovalbumin. Ovalbumin is incorporated as a candidate ingredient of immunogenic compositions comprising cross-beta structure. The ovalbumin is either serving as the antigen itself, to which an immune response should be directed, or ovalbumin is used as the cross-beta adjuvant part in immunogenic compositions, comprising a target antigen with a different amino-acid sequence. For this latter use, ovalbumin comprising cross-beta is combined with the target antigen, to which an immune response is desired. Cross-beta adjuvated ovalbumin is for example covalently coupled to the antigen of choice, using coupling techniques known to a person skilled in the art. When ovalbumin is the target antigen itself, non-treated ovalbumin and cross-beta-adjuvated ovalbumin are used in a similar way, in immunogenic composition preparations.

Lyophilized ovalbumin, or chicken egg-white albumin (OVA, Sigma, A5503 or A7641) is dissolved as follows. OVA is gently dissolved at indicated concentration in phosphate buffered saline (PBS; 140 mM sodium chloride, 2.7 mM potassium chloride, 10 mM disodium hydrogen phosphate, 1.8 mM potassium dihydrogen phosphate, pH 7.3; local pharmacy), avoiding any foam formation, stirring, vortexing or the like. OVA is dissolved by gently swirling, 10 minutes rolling on a roller device, 10 minutes warming in a 37° C.-water bath, followed by 10 minutes rolling on a roller device. Aliquots in Eppendorf tubes are frozen at −80° C. Before use, OVA solution is either prepared freshly, or thawed from −80° C. to 0° C., or after thawing kept at 37° C. for 30 minutes. Furthermore, an OVA solution is applied to an endotoxin affinity matrix for removal of endotoxins present in the OVA preparation. Before and after applying OVA to the matrix, endotoxin levels are determined using an Endosafe apparatus (Charles River), and/or using a chromogenic assay for determining endotoxin levels (Cambrex), both using Limulus Amoebocyte Lysate (LAL). Misfolded OVA, termed “dOVA,” is prepared as indicated below (see Section “Protocols for introducing cross-beta in proteins”).

Hemagglutinin 5 protein of H5N1 virus strain A/Hong kong/156/97. Hemagglutinin 5 protein (H5) of H5N1 virus strain A/Hong kong/156/97 (A/HK/156/97) is expressed in 293 cells with a C-terminal FLAG tag and His tag, and purified using Ni²⁺-based affinity chromatography as described in patent application WO/2007/008070. In addition, the recombinantly produced H5-FLAG-His construct is purified using affinity chromatography with the anti-FLAG antibody M2 immobilized on a matrix (Sigma, A2220), according to the manufacturer's recommendations and using FLAG peptide (Sigma, F3290) for elution of H5-FLAG-His from the matrix. Protein solutions are stored at −80° C. for a long term and after micro filtration at 4° C., for a short term. In this example, upon purification using anti-FLAG antibody based affinity chromatography, two batches of H5 were obtained. One batch of H5-FLAG-His is termed non-treated H5, batch 2 (“nH5-2,” concentration 30 μg/ml). A second batch of H5-FLAG-His was subsequently subjected to size-exclusion chromatography (SEC) using a HiLoad 26/60 Superdex 200 column on an Äkta Explorer (GE Healthcare; used at the ABC-protein expression facilities of the University of Utrecht, Dr R. Romijn & Dr. W. Hemrika). For this purpose, H5-FLAG-His solution in PBS is concentrated on Macrosep Centrifugal Devices 10K Omega (Pall Life Sciences) or CENTRIPREP Centrifugal Filter Devices YM-300 (Amicon). Running buffer was PBS. The H5 batch after the SEC run, termed non-treated H5, batch 1 (“nH5-1”), was stored at 4° C. after micro filtration (concentration 400 μg/ml, as determined with the BCA method). This batch nH5-1 is used for misfolding procedures described below.

H5 of H5N1 strain A/Vietnam/1203/04. H5 of H5N1 strain A/Vietnam/1203/04 (A/VN/1203/04) is purchased from Protein Sciences, and consists mainly of HA2, with relatively lower amounts of HA1 and HA0. Purity is 90%, as determined with densitometry, according to the manufacturer's information. Buffer and excipients are 10 mM sodium phosphate, 150 mM NaCl, 0.005% Tween80, pH 7.2. The H5 concentration is 922 μg/ml (lot 45-05034-2) or 83 μg/ml (lot 45-05034RA-2). This non-treated H5 is termed “nH5” and stored at 4° C. or at −80° C.

Other antigens. The proteins described above are used for preparation of immunogenic compositions. However, the disclosed technologies are by no means restricted to the generation of immunogenic compositions comprising OVA, FVIII, H5 of A/VN/1203/04 or A/HK/156/97, or E2. Examples that further disclose the described technologies and their applications, are also generated using other and/or additional peptides, polypeptides, proteins, glycoproteins, protein-DNA complexes, protein-membrane complexes and/or lipoproteins as a basis for immunogenic compositions. These peptides, polypeptides, proteins, glycoproteins, protein-DNA complexes, protein-membrane complexes and/or lipoproteins are the antigen component, the cross-beta-adjuvated component or both the antigen component and the cross-beta-adjuvated component of immunogenic compositions. The peptides, polypeptides, proteins, glycoproteins, protein-DNA complexes, protein-membrane complexes and/or lipoproteins are originating from amino-acid sequences unrelated to pathogens and/or diseases, when used as the cross-beta-adjuvated ingredient of an immunogenic composition, or are originating from amino-acid sequences that are related to and/or involved in and/or are part of pathogens, tumors, cardiovascular diseases, atherosclerosis, amyloidosis, autoimmune diseases, graft-versus-host rejection and/or transplant rejection, when they are part of the target antigen and/or are the cross-beta-adjuvated ingredient of an immunogenic composition. In fact, the disclosed technologies are applicable to any amino-acid sequence, either of the antigen, or of the cross-beta-adjuvant.

Non-limiting examples of peptides, polypeptides, proteins, glycoproteins, protein-DNA complexes, protein-membrane complexes and/or lipoproteins that are used as antigen and/or as cross-beta-adjuvant are, for example, virus surface proteins, bacterial surface proteins, pathogen surface exposed proteins, gp120 of HIV, proteins of human papilloma virus, any of the neuramidase proteins or hemagglutinin proteins or any of the other proteins of any influenza strain, surface proteins of blue tongue virus, proteins of foot- and mouth disease virus, bacterial membrane proteins, like for example PorA of Neisseria meningitides, oxidized low density lipoprotein, tumor antigens, tumor specific antigens, amyloid-beta, antigens related to rheumatoid arthritis, B-cell surface proteins CD19, CD20, CD21, CD22, proteins suitable for serving as target for immunocastration, proteins of hepatitis C virus (HCV), proteins of respiratory syncytial virus (RSV), proteins specific for non small cell lung carcinoma, malaria antigens, proteins of hepatitis B virus.

### Protocols and Procedures for Misfolding Proteins and Introducing Cross-Beta in Proteins

Peptides, polypeptides, proteins, glycoproteins, protein-DNA complexes, protein-membrane complexes and/or lipoproteins, in summary referred to as “protein” throughout this section, are misfolded with the occurrence of cross-beta structure after subjecting them to various cross-beta-inducing procedures. Below, a summary is given of a non-limiting series of those procedures, which are preferably applied to the proteins used in immunogenic compositions.

Misfolding of proteins with the occurrence of cross-beta is induced using selected combinations of several parameters. The following parameters settings are applied for proteins:
a. protein concentrations ranging from 10 μg/ml to 30 mg/ml, and preferably between 25 μg/ml and 10 mg/ml,
b. pH between 0 and 14, and preferably at pH 1.5-2.5 and/or pH 6.5-7.5 and/or 11.5-12.5 and or at the iso-electric point (IEP) of a protein, and for example induced with HCl or NaOH, for example using 2-5 M stock solutions.
c. NaCl concentrations between 0 and 5000 mM, and preferably 125-175 mM
d. buffer selected from PBS, HEPES-buffered saline (20 mM HEPES, 137 mM NaCl, 4 mM KCl, pH 7.4), or no buffer (H₂O),
e. a reducing agent like dithiothreitol (DTT) or β-mercaptoethanol is incorporated in the reaction mixture, and
f. temperature gradients and temperature end-points for an indicated time frame, that are applied for selected time frames of 10 seconds up to 24 h, and with selected ranges between 0 and 120° C., and preferably between 4 and 95° C., with preferably steps of 0.1-5° C./minute for gradients.

Furthermore, protein misfolding is induced for example by, but not limited to, post-translational modifications like for example glycation, using for example carbohydrates, oxidation, using for example CuSO₄, citrullination, using for example using peptidylarginine deiminases, acetylation, sulfatation, (partial) de-sulfatation, (partial) de-glycosylation, enzymatic cleavage, polymerization, exposure to chaotropic agents like urea (for example 0.1-8 M) or guanidinium-HCl (for example 0.1-7 M).

Misfolding of proteins with appearance of cross-beta is also achieved upon subjecting proteins to exposure to adjuvants currently in use or under investigation for future use in immunogenic compositions. Proteins are exposed to adjuvants only, or the exposure to adjuvants is part of a multi-parameter misfolding procedure, designed based on the aforementioned parameters and conditions. Non-limiting examples of adjuvants that are implemented in protocols for preparation of immunogenic compositions comprising cross-beta are alum (aluminium-hydroxide and/or aluminium-phosphate), MF59, QS21, ISCOM matrix, ISCOM, saponin, QS27, CpG-ODN, flagellin, virus like particles, IMO, ISS, lipopolysaccharides, lipid A and lipid A derivatives, complete Freund's adjuvant, incomplete Freund's adjuvant, calcium-phosphate, Specol.

A typical method for induction of cross-beta conformation in a protein is designed as follows in a matrix format, from which preferably subsets of parameter settings are selected.
i. protein concentration is 40/200/1000 μg/ml
ii. pH is 2, 7, 12 and at the IEP of the protein
iii. DTT concentration is 0 or 200 mM
iv. NaCl concentration is 0 or 150 mM
v. urea concentration is 0/2/8 M
vi. buffer is PBS or HBS (with adjusted NaCl concentration and/or pH, when indicated)
vii. temperature gradient is
   a. constantly at 4° C./22° C.-37° C./65° C. for an indicated time
   b. from room temperature to 65° C./85° C., for 1 to 5 cycles

Subsets of selected parameter settings are for example as follows.
A. 1 mg/ml protein in PBS, pH 7.3, 200 mM DTT, 150 mM NaCl, kept at 37° C. for 60 minutes
B. 200 μg/ml protein in PBS, 150 mM NaCl, heated in a cyclic manner for three cycli from 25° C. to 85° C., at 0.5° C./minute, with varying pHs.

Misfolding of E2. E2 protein is misfolded accompanied by introduction of cross-beta, by applying various parameter ranges, selected from described parameters a-f (see above). For example, E2 concentration ranges from 50 μg/ml to 2 mg/ml; selected pH is 2, 7.0-7.4 and 12; selected NaCl concentration is 0-500 mM, for example 0/50/150/500 mM; selected buffer is PBS or HBS or no buffer (H₂O); selected temperature gradient is for example as described for OVA, below. For example, E2 at approximately 300 μg/ml in PBS, heated in PCR cups in a PTC-200 thermal cycler (MJ Research, Inc.): 25° C. for 20 seconds and subsequently heated (0.1° C./second) from 25° C. to 85° C. followed by cooling to 4° C. for 2 minutes. This cycle is for example repeated twice (total number of cycles is 3). For example, E2 is subsequently stored at −20° C.

For the examples described below, non-treated E2 (nE2) at approximately 280 μg/ml in PBS was incubated at 25° C. for 20 seconds and was subsequently gradiently heated (0.1° C./second) from 25° C. to 85° C. followed by cooling at 4° C. for 2 minutes. This cycle was repeated twice and then, the E2 solution, referred to as cross-beta E2 (cE2) was stored at −20° C.

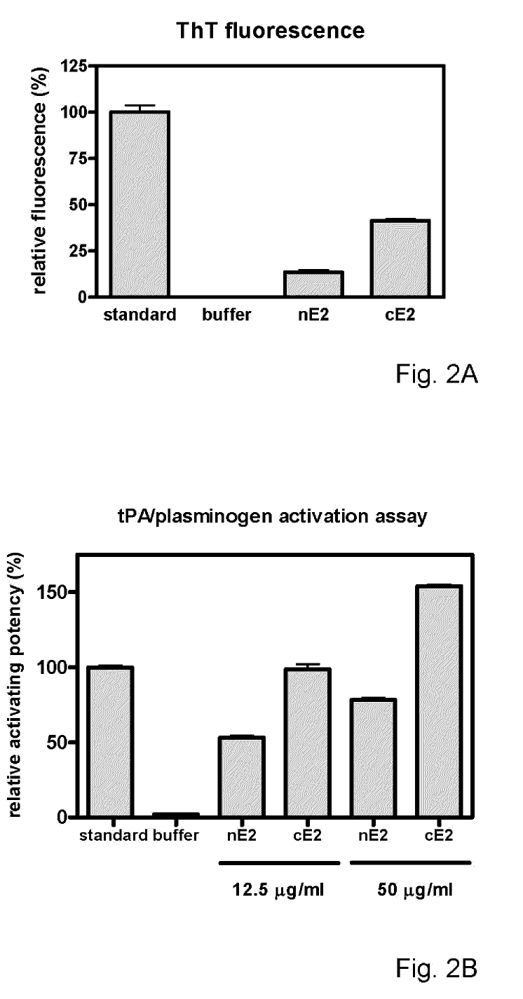

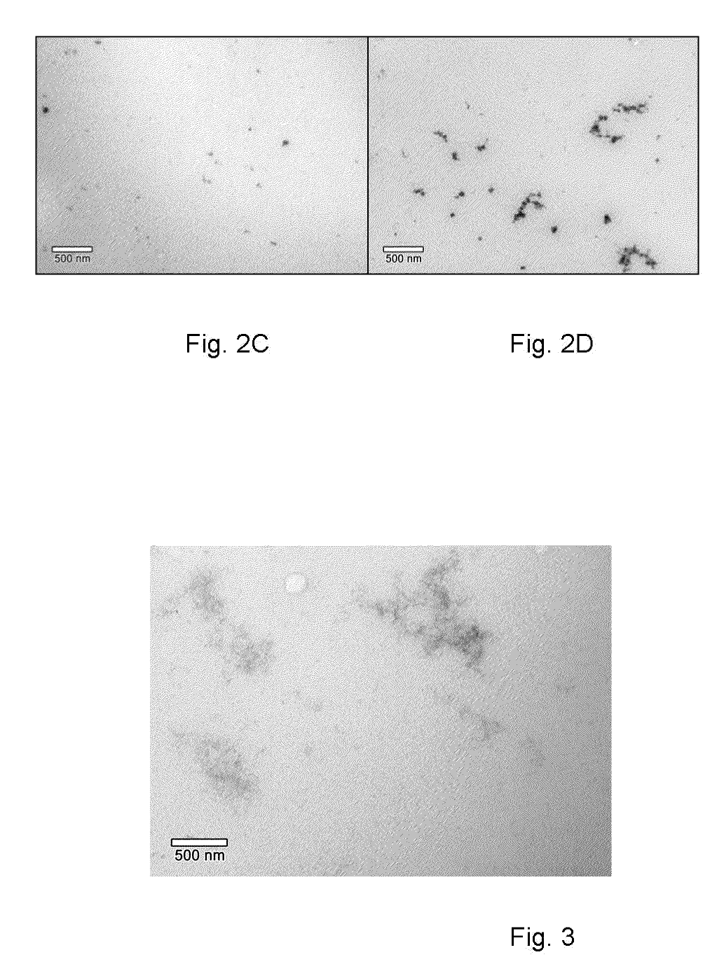

Structural differences and differences in cross-beta content between nE2 and cE2 were assessed using ThT fluorescence measurement, tPA/Plg activation analysis and TEM imaging. See FIG. 2. From these graphs and figures it is clearly seen that the content of cross-beta in cE2 is increased when compared to nE2; both ThT fluorescence and tPA/Plg activating potential are increased. On the TEM images it is seen that cE2 appears as clustered and relatively large multimers with various sizes, whereas also nE2 displays assemblies of protein, though with smaller size and not clustered. Further analysis of cross-beta content and appearance, and further analysis of multimeric size and multimeric size distribution is assessed by subjecting the E2 samples to various of the aforementioned analyses for cross-beta determination and molecular structure and size determinations. Furthermore, various additional appearances of cE2 variants are generated by subjecting nE2 and/or nE2-FLAG-His to selected misfolding procedures as depicted above. For example, nE2 is used at 0.1 and 1 mg/ml, at pH 2/7/12, with/without DTT, for cyclic heat-gradients running from 4 to 85° C., for 1 to 5 cycles, resulting in 60 variants of cE2. These variants are subjected to analysis of binding of antibodies, for selecting those cE2 variants that combine the ability to bind functional antibodies (see below) with the presence of potent immunogenic cross-beta conformation. In addition, nE2 is for example coupled to dOVA standard and/or a different variant of misfolded OVA with proven potent cross-beta-adjuvating properties (see the section on OVA misfolding and OVA immunizations).

Misfolding of OVA. OVA is for example misfolded with introduction of cross-beta using the following misfolding procedures:
1. 10 mg/ml OVA in PBS, heating from 25 to 85° C., 5° C./minute
2. 1 mg/ml OVA in PBS, heating from 25 to 85° C., 5° C./minute
3. 0.1 mg/ml OVA in PBS, heating from 25 to 85° C., 5° C./minute
4. 10 mg/ml OVA in HBS, heating from 25 to 85° C., 5° C./minute
5. 1 mg/ml OVA in HBS, heating from 25 to 85° C., 5° C./minute
6. 0.1 mg/ml OVA in HBS, heating from 25 to 85° C., 5° C./minute
7. similar to the above six methods 1-6, now with a cooling step from 85° C. back to 25° C., and again heating to 85° C. (repeated twice)
8. similar to the above six methods 1-6, now with a heating rate of 0.1° C./minute, and a cooling step from 85 back to 25° C. (1-5 cycles)
9. addition of a final concentration of 1% SDS to 1 mg/ml OVA; incubation at room temperature for 30 minutes-16 h
10. addition of urea to 0.1-10 mg/ml OVA, to a final concentration of 2-8 M. Incubation for preferably 1-16 h at preferably 4-65° C. OVA solution is dialyzed against preferably H₂O or PBS or HBS, before further use.
11. constantly heating of preferably 0.1-10 mg/ml OVA in preferably PBS or HBS or H₂O, for preferably 1-72 h at preferably 4-100° C. For example 0.1 and 1 mg/ml in PBS, for 20 h at 65° C.
12. constantly heating of preferably 0.1-10 mg/ml OVA in PBS, for 10 minutes at 100° C. For example 0.1 and 1 and 10 mg/ml.
13. addition of a final concentration of 0.5% SDS to 1 mg/ml OVA; incubation for preferably 1-16 h at preferably 4-37° C., for example 1 h at room temperature.
14. Oxidation: addition of CuSO₄ to a final concentration of 1 mM and incubation for 24 h at 37° C. The oxidized OVA is dialyzed before further use.
15. incubation of 300 μg/ml OVA with 4 mM ascorbic acid, 40 μM CuCl₂, for 3 h, in NaPi buffer pH 7.4. Oxidation is stopped by adding EDTA from a 100 mM stock, to 1 mM final concentration. The oxidized OVA is dialyzed before further use.
16. pH of an OVA solution at 600 μg/ml in HBS is lowered to pH 2 by adding a suitable amount of HCl from a 5 M stock. The solution is subsequently kept at 37° C. for 30 minutes. Then, the pH is adjusted with NaOH to pH 7-7.4.
17. pH of an OVA solution at 600 μg/ml in HBS is raised to pH 12 by adding a suitable amount of NaOH solution from a 5 M stock. The solution is subsequently kept at 37° C. for 30 minutes. Then, the pH is adjusted with HCl back to pH 7-7.4.
18. For comparison with methods 16 and 17, the same final amount of NaCl is added, which is finally added to the solutions described in 16 and 17 by adding HCl/NaOH or NaOH/HCl, to OVA solution, after incubation for 30 minutes at 37° C.

OVA was subjected to the following misfolding procedure for inducing cross-beta conformation. OVA was dissolved in PBS to a concentration of 1.0 mg/ml. The solution was put on a roller device for 10 minutes at room temperature (RT), than 10 minutes at 37° C. in a water bath and subsequently again for 10 minutes on the roller device (RT). Then, 200 μl aliquots of OVA solution was heat-treated in a PTC-200 PCR machine (MJ Research) as follows: five cycles of heating from 30° C. to 85° C. at 5° C./minute; cooling back to 30° C. After five cycles misfolded OVA, termed dOVA, was cooled to 4° C. and subsequently stored at −80° C. This preparation of dOVA is used as a standard reference, termed “standard,” with cross-beta content that results in a maximal signal (arbitrarily set to 100%) in indicated cross-beta detecting assays, at a given concentration.

Cross-beta analyses are performed with dOVA standard at a regular basis in our laboratories. For example in FIGS. 2, 6, 7 and 10, dOVA standard is analyzed for its capacity to enhance ThT fluorescence, Congo red fluorescence, tPA/Plg activation. Furthermore, dOVA standard appears as clusters or strings of aggregated molecules with various sizes on TEM images (FIG. 3). Further cross-beta analyses and multimeric distribution analyses using described methods are applied to the dOVA standard preparation and to additionally produced misfolded OVA variants, as depicted above.

Misfolding of H5 of H5N1 strain A/HK/156/97. The H5-FLAG-His batch nH5-1, obtained after anti-FLAG antibody affinity chromatography and size exclusion chromatography, was subjected to two misfolding procedures.
A. A batch of 2 mg of nH5-1 (400 μg/ml in PBS, filtered through a 0.22 μm filter) was misfolded as follows. Aliquots of 120 μl of nH5-1 in PCR strips were incubated at 25° C. for 20 seconds and subsequently heated (0.1° C./second) from 25° C. to 85° C., followed by cooling at 4° C. for 2 minutes. This cycle was repeated twice. Then, the H5 sample was pooled and stored at 4° C., and referred to as “CH5-A.”
B. A second batch of 2 mg of nH5-1 was subjected to the following misfolding procedure. DTT was added from a sterile 1 M stock in H₂O to a final concentration of 100 mM. The sample was mixed by vortexing, and incubated for 1 h at 37° C. (stove). Subsequently, the H5 samples was dialyzed three times for ˜3 h against 3 l PBS under sterile conditions, at 4° C. For dialysis, Slide-a-lyzers with a molecular weight cut-off of <10 kDa (Pierce) were used. The volume of the H5 sample, referred to as “CH5-B,” after recovery was unchanged with respect to the starting volume.

For structure analyses and for formulation of vaccine candidate solution, before use the nH5-1 and nH5-2 were centrifuged for 10 minutes at 16,000*g at room temperature. CH5-A and CH5-B were used without the centrifugation step.

The nH5-1 and CH5-B samples were analyzed on an analytical SEC column (U-Express Proteins, Utrecht, NL). For this purpose, approximately 80 μl of the 400 μg/ml stocks was applied to a Superdex200 10/30 column, connected to an Äkta Explorer (GE Healthcare). Running buffer was PBS. Samples were centrifuged for 20 minutes at 13,000*g before loading onto the column. The samples were run at a flow rate of 0.2 ml/minute and elution of protein was recorded by measuring absorbance at 280 nm.

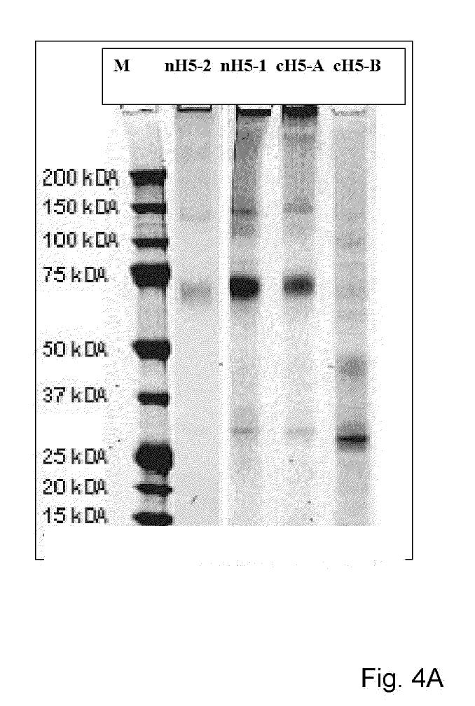

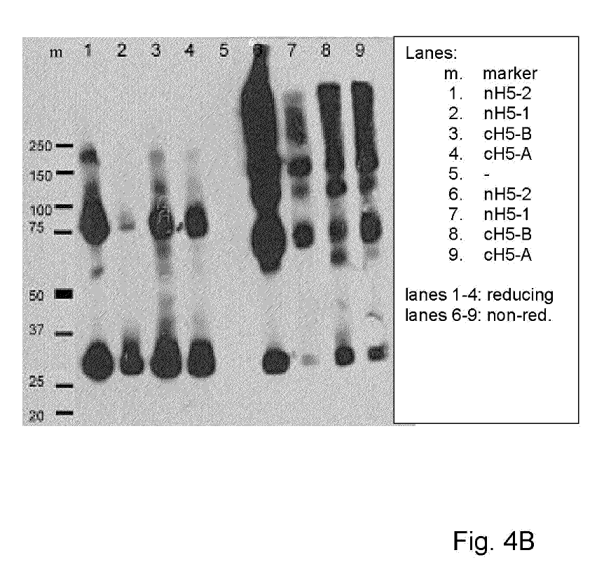

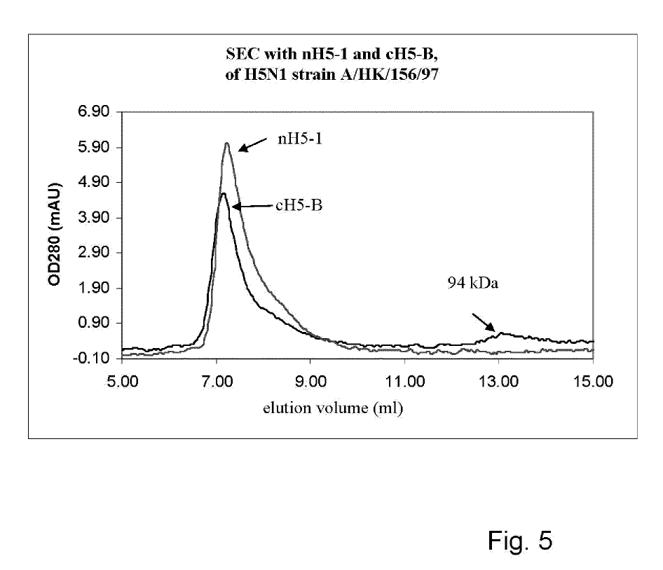

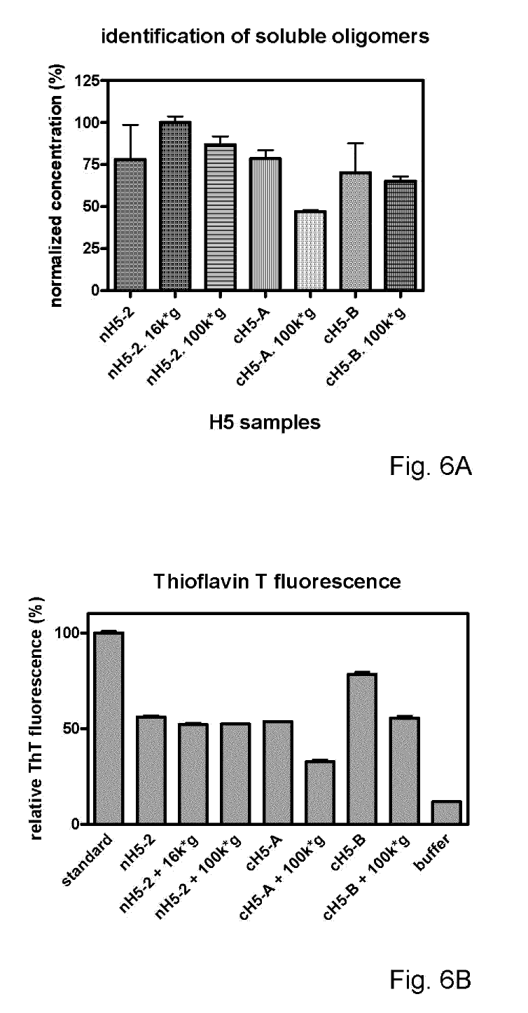

The nH5-1 and nH5-2 preparations appear on SDS-PA gel and Western blot as multimers ranging from monomer up till aggregates that do not enter the gel (FIG. 4). Upon treatment with DTT, these multimers monomerize, indicative for the covalent coupling of nH5 molecules through disulfide bonds (See FIG. 4B). The CH5-A preparation appears with a similar pattern on gel and blot compared to the non-treated variants (FIG. 4). In contrast, the CH5-B variant appears predominantly as monomers on gel and blot, with also dimers and oligomers present, but to a far lesser extent than seen in nH5-1, nH5-2 and CH5-A (FIG. 4). This observation is reflected in the elution patterns of nH5-1 and CH5-B from the SEC column, depicted in FIG. 5. The nH5-1 elutes as one peak in the flow-through of the column, whereas CH5-B elutes predominantly as a peak in the flow-through with a small peak at approximately the H5 monomer size. In conclusion, it appears that CH5-B comprises predominantly multimers that are more readily separated into smaller multimers and monomers, when compared to nH5-1, nH5-2 and CH5-A. Ultracentrifugation for 1 h at 100,000*g, which is used as a method to separate soluble oligomers of proteins from multimers that are precipitated in the pellet fraction, was applied to nH5-1, CH5-A and CH5-B (FIG. 6). It appears that when nH5-1 is subjected to the g-forces, no molecules that contribute to the ThT fluorescence are pelleted, indicative for the presence of soluble oligomers comprising cross-beta, and the absence of insoluble aggregates with cross-beta. In contrast, by applying 100,000*g for 1 h on CH5-A and CH5-B, a fraction of the ThT fluorescence enhancement is lost, indicative for the removal of insoluble multimers with cross-beta from the solution. The remaining fraction of both H5 variants apparently comprises soluble multimers with cross-beta conformation. TEM images of nH5-1, nH5-2 and CH5-A, as depicted in FIG. 6, show that all three H5 variants comprise multimers to a certain extent. The nH5-2 concentration is about 13-fold lower than the nH5-1 and CH5-A concentration, reflected in the lower density of multimers. When comparing nH5-1 and CH5-A, it is observed that CH5-A comprises less multimers but a higher number of larger multimers. These analyses of multimer size and size distribution are extended using more of the aforementioned techniques, and by incorporating more appearances of H5 after subjecting H5 solutions to various alternative misfolding procedures.

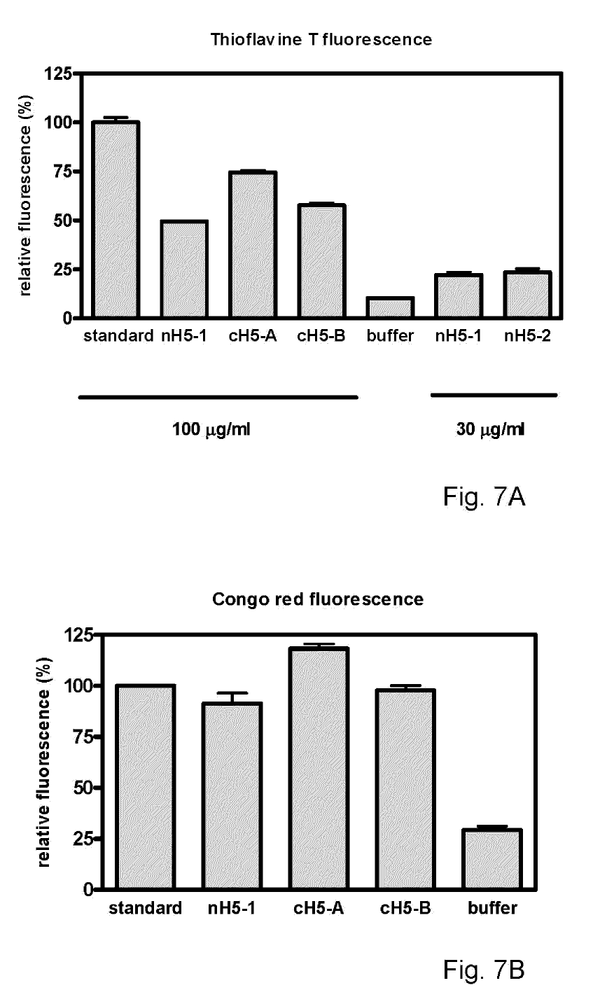

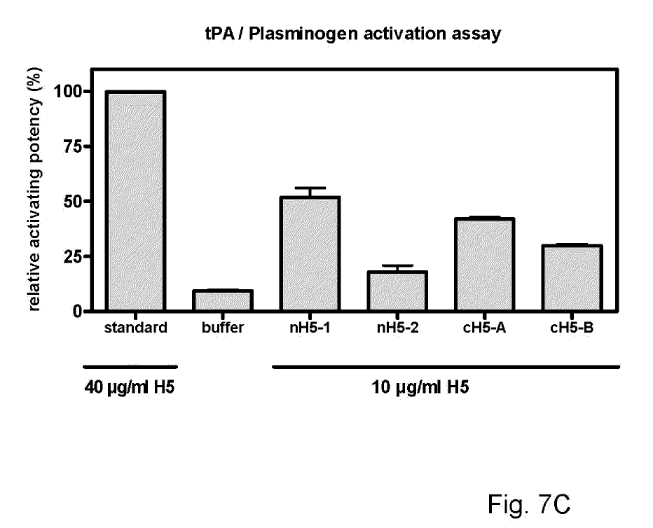

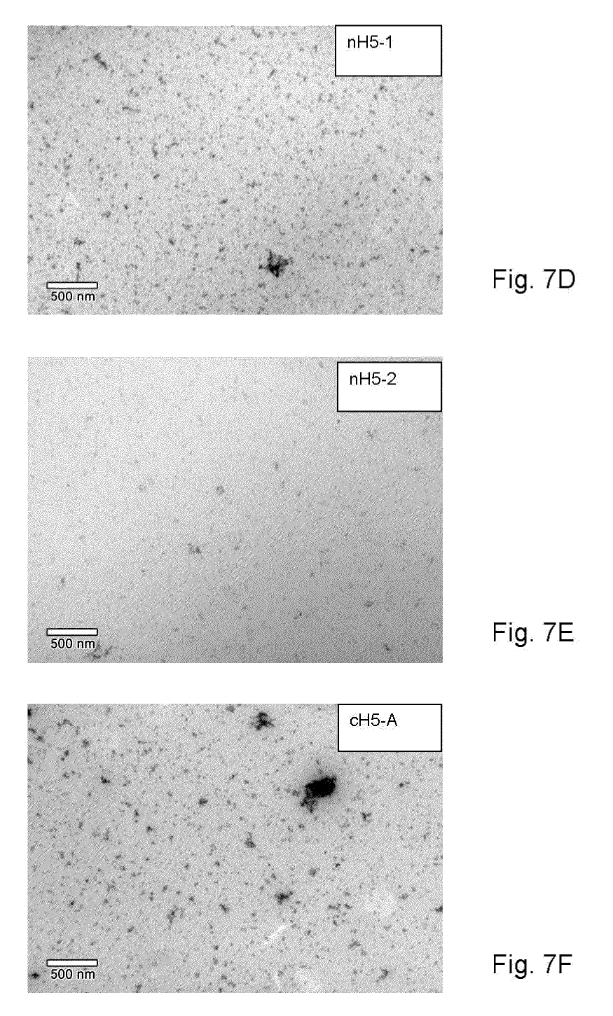

The nH5-1 and nH5-2 preparations comprise a considerable amount of cross-beta conformation, as depicted in FIG. 7, showing ThT fluorescence enhancement, Congo red fluorescence enhancement and the ability to increase tPA/Plg activity for both non-treated H5 variants. When comparing CH5-A with CH5-B it is clear that CH5-A displays higher signals in the three cross-beta detecting assays. When comparing the patterns of the signals obtained in the three assays with the four H5 variants, it is seen that all four variants display a unique combination of signals, indicating that four different appearances and/or contents of cross-beta are present. H5 variants are subjected to further cross-beta analyses in order to obtain more insight in the different appearances of cross-beta upon subjecting H5 to varying misfolding conditions.

Misfolding of H5 of H5N1 strain A/VN/1203/04. H5 of H5N1 strain A/VN/1203/04, as obtained from Protein Sciences, was subjected to four misfolding procedures, as indicated below.

1. nH5

For comparison, NaCl from a 5 M stock was added to non-treated H5 stock (922 μg/ml, 4° C., 150 mM NaCl), to a final concentration of 171 mM NaCl, and subsequently aliquoted in Eppendorf cups and stored at −20° C. Endotoxin level: <0.05 EU/10 μg/ml solution nH5 (determined using an Endosafe pts apparatus (Charles River). The solution was clear and colorless. For structure analyses and for formulation of vaccine candidate solution, before use the nH5 was centrifuged for 10 minutes at 16,000*g at room temperature.

### 2. CH5-1

Aliquots of nH5 in PCR strips (Roche) were incubated at 25° C. for 20 seconds and subsequently gradiently heated (0.1° C./second) from 25° C. to 85° C. followed by cooling back to 4° C., and kept at 4° C. for 2 minutes. This heat cycle was repeated twice. Then, aliquots in Eppendorf 500 μL cups were stored at −20° C. Code: “CH5-1.” The preparation CH5-1 was slightly turbid with some visible precipitates after heat treatment.

### 3. CH5-2

The pH of the nH5 stock kept at 4° C., was lowered to pH 2 by adding HCl from a 15% v/v stock. Then, aliquots of 100 μL/cup in PCR strips were heated in a PTC-200 thermal cycler, as follows. The samples were incubated at 25° C. for 20 seconds and subsequently gradiently heated (0.1° C./second) from 25° C. to 85° C. followed by cooling back to 4° C., and kept at 4° C. for 2 minutes. This heat cycle was repeated twice. Subsequently, the pH was adjusted to pH 7 by adding a volume NaOH solution from a 5 M stock. Then, aliquots in Eppendorf 500 μL cups were stored at −20° C. Code: “CH5-2.” The solution was clear and colorless.

### 4. CH5-3

The pH of nH5 kept at 4° C., was elevated to pH 12 by adding a volume NaOH solution from a 5 M stock. Then, aliquots of 100 μL/cup in PCR strips were treated as follows in a PTC-200 thermal cycler. The samples were incubated at 25° C. for 20 seconds and subsequently gradiently heated (0.1° C./second) from 25° C. to 85° C. followed by cooling back to 4° C., and kept at 4° C. for 2 minutes. This heat cycle was repeated twice. Subsequently the pH was adjusted to pH 7 by adding a volume HCl solution from a 5 M stock. Then, aliquots in Eppendorf 500 μL cups were stored at −20° C. Code: “CH5-3.” The solution was clear and colorless.

### 5. CH5-4

D-Glucose-6-phosphate disodium salt hydrate (g6p, Sigma; G7250) was added from a 2 M stock in PBS to nH5 to a final concentration of 100 mM g6p (20-fold dilution). Then it was incubated for 67 h at 80° C. The solution was intensively dialyzed against PBS, aliquoted in Eppendorf 500 μL cups, and stored at −20° C. The solution was light brown with white precipitates, visible by eye.

For structure analyses and for formulation of vaccine candidate solution, before use the nH5 was centrifuged for 10 minutes at 16,000*g at room temperature. CH5-1 to 4 were used without the centrifugation step.

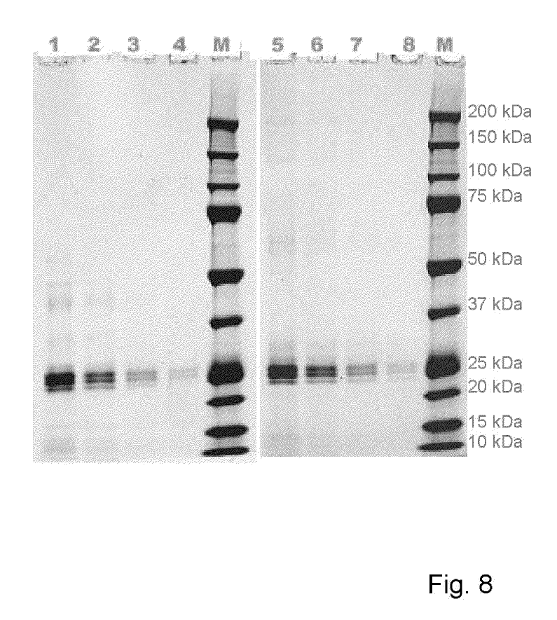

The nH5 protein, as purchased from Protein Sciences, appears predominantly as the approximately 25 kDa HA2 fragment, with a smaller content of HA0 (full-length H5) and HA1 (molecular weight approximately 50 kDa) on reducing and non-reducing SDS-PA gels, stained with Coomassie (FIG. 8).

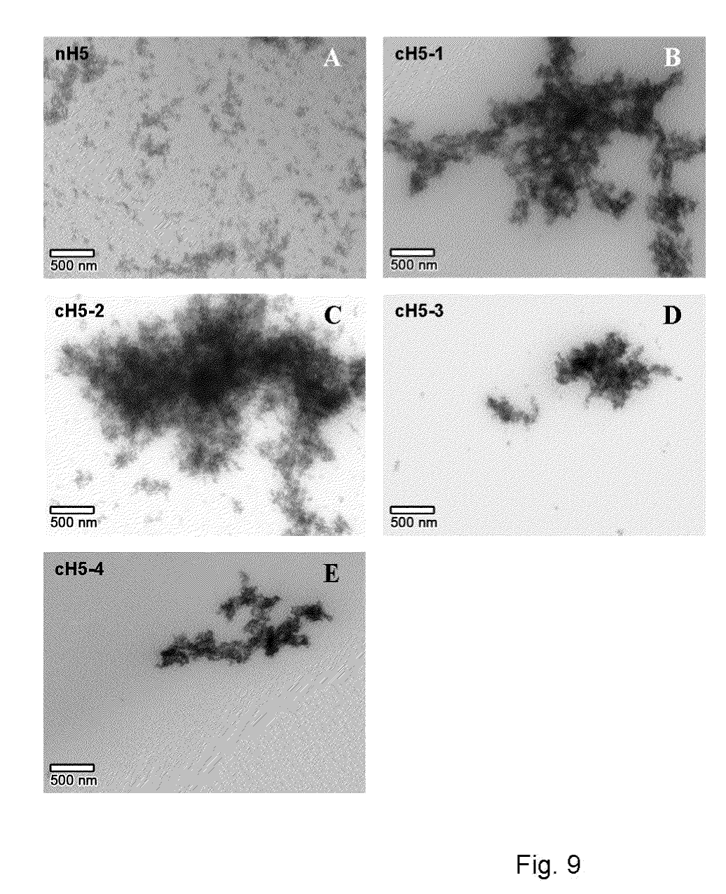

The nH5 appears on a TEM image as amorphous multimers which are relatively small in size and which tend to aggregate into clusters, as seen in the supernatant after 10 minutes centrifugation at 16,000*g (FIG. 9). In contrast, the four misfolded forms of H5, CH5-1 to 4, all appear as larger aggregates. The aggregates observed for CH5-1 and CH5-2 are similar in size and larger than the aggregates seen for CH5-3 and 4. Aggregates in CH5-2 seem to be more amorphous than the aggregates seen in CH5-2.

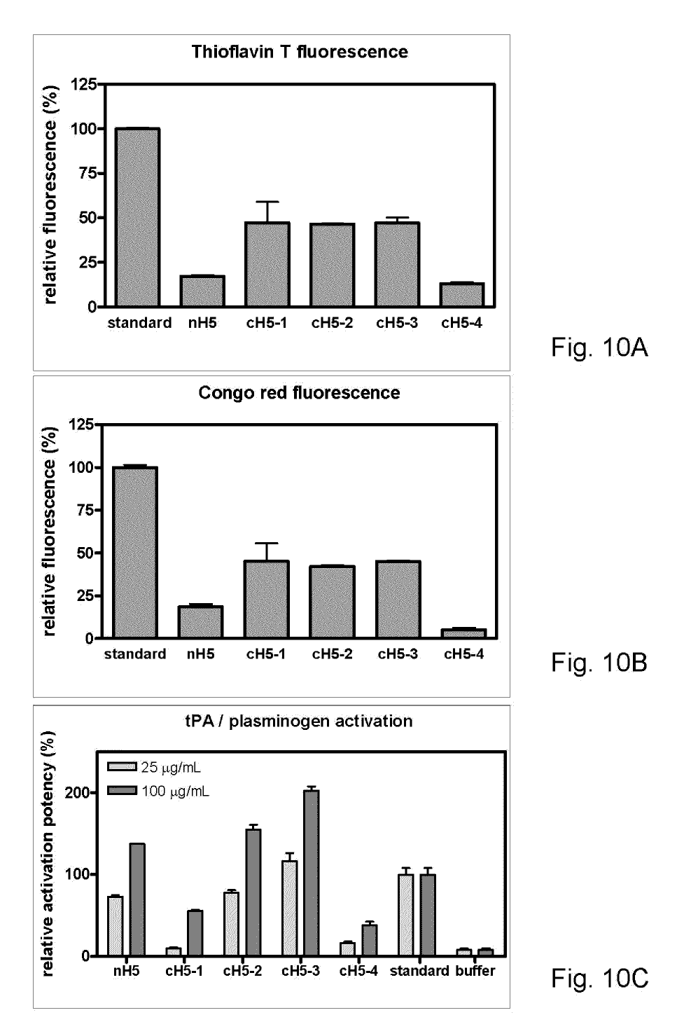

ThT fluorescence is enhanced with CH5-1 to 3, when compared to nH5 (FIG. 10A). The non-treated nH5 still displays a significant ThT fluorescent signal. The signal is decreased for CH5-4, when compared to nH5. A similar pattern is seen for Congo red fluorescence (FIG. 10B). The relative tPA/Plg activation potency of nH5 and CH5-1 to 4 displays a different pattern. CH5-2 and 3 enhance tPA/Plg activation to a somewhat larger extent than nH5, whereas CH5-1 and CH5-4 are less potent activators of tPA/Plg when compared to nH5 (FIG. 10C). The five H5 forms are subjected to extended cross-beta analyses and extended multimer size and distribution analyses, in order to obtain more detailed information about the structural appearances.

All of the aforementioned antigens are preferably subjected to the described Disappearing Epitope Scanning approach for obtaining an immunogenic composition that comprises cross-beta and T-cell epitope motifs.

### Example

### T Cell Activation by Antigen Comprising a T Cell Epitope and at Least One Cross-Beta Structural Element

This example illustrates the ability to generate and selected an immunogenic compound comprising a cross-beta structure and a T cell epitope capable of inducing a T cell response. The selected immunogenic compounds were able of inducing an immune response that delayed tumor growth more efficient.

Study design. Ovalbumin was used as test protein and antigen. Studies were performed using either a T cell clone, DO11.10, T cells (naive), OT-I and OT-II, isolated from transgenic mice or T cells (primed in vivo) isolated from mice immunized with untreated OVA, comprising few cross-beta structural elements or with OVA comprising increased numbers of cross-beta structural elements. Cross-beta structural elements were induced in three different ways. Activation of T cells was determined in several ways, such as increased secretion of IL-2 by DO11.10 cells, proliferation of naive T cells or secretion of IFNγ by OT-I or OT-II cells, proliferation of primed T cells isolated from OVA-immunized mice or IFNγ production by T cells isolated from OVA-immunized mice. The efficacy of the immunogenic composition in inducing a T cell response and the efficacy of the response to delay tumor growth was determined using T cells isolated from mice immunized with different OVA-immunogenic compositions comprising cross-beta structure, and compared with an immunogenic composition comprising a relative low content of cross-beta structure in OVA.

Preparation of cross-beta variants of OVA. Four Different Forms of Ova comprising cross-beta structure, termed nOVA, dOVA-1, dOVA-2 and dOVA-3, were prepared according to examples of procedures to induce cross-beta structure described in this application and described below, and were compared in this example.

nOVA. OVA was dissolved in PBS to a concentration of 1.0 mg/mL. The solution was kept for 20 min at 37° C. in a water bath and subsequently for 10 min on the roller device (at room temperature). Aliquots were stored at −80° C. This form of OVA form, comprising relatively low levels of cross-beta structure is referred to as nOVA, cross-beta nOVA or nOVA standard.

Method for inducing cross-beta structure: dOVA-1. OVA was dissolved at 5.2 mg/ml in HBS buffer (20 mM Hepes, 137 mM NaCl, 4 mM KCl). To dissolve OVA the solution was incubated for 20 min in a water bath at 37° C. and 10 min on a roller device at RT. The solution appeared clear. 5 M HCl is added to 2% of the total volume. The solution was mixed by swirling. The solution was incubated for 40 minutes at 37° C. (water bath). The solution appeared white/turbid. 5 M NaOH stock (2% of the volume) was added to neutralize the solution. The solution was mixed by swirling. The visual appearance of the solution remained turbid. Samples were aliquoted and stored at −80° C.

Method for inducing cross-beta structure: dOVA-2. OVA was dissolved in PBS to a concentration of 1.0 mg/mL. The solution was kept for 20 min at 37° C. in a water bath and subsequently for 10 min on the roller device (at room temperature). 200 μl aliquots in PCR cups were heat-treated in a PCR machine (MJ Research, PTC-200) (from 30° C. to 85° C. in steps of 5° C. per min). This cycle was repeated 4 times (in total 5 cycles). The samples were subsequently cooled to 4° C. The solutions were pooled, divided in 100 μL aliquots and stored at −80° C.

Method for inducing cross-beta structure: dOVA-3. OVA was dissolved in PBS to a concentration of 1 mg/ml and subsequently incubated for 10 minutes at 37° C. followed by 10 minutes RT incubation on a roller device. 200 μL aliquots were incubated in PCR strips (total 5.5 mL) at 75° C. in MyiQ real time PCR, BIORAD ΔT=1 minute at 25° C., 25° C. to 75° C., ramp rate 0.1° C./second, incubation time approximately 16 h at 75° C., without cooling.

Endotoxin measurement. The endotoxin content of OVA was measured at 20 μg/mL (diluted in sterile PBS). The Endosafe cartridge had a sensitivity of 5-0.05 EU/mL (Sanbio, NL). The endotoxin level are shown in table 1. The endotoxin level of the dilution buffer PBS is checked regularly and is below 0.050 EU/mL. Mice were immunized with 5 μg OVA per mouse. The amount of endotoxins in 5 μg is calculated from the endotoxin level determined at 20 μg/mL.

### Structural Analysis of OVA Variants

Visual inspection by eye and under a microscope, of various OVA forms. Table 2 describes the appearance of nOVA and the different dOVAs by eye. It is observed that dOVA-1 and dOVA-3 comprise insoluble OVA multimers as the solution is no longer clear upon treatment.

Transmission electron microscopy imaging (TEM) with OVA forms. The various OVA forms are subjected to TEM analysis. Table 3 summarizes the analysis. It is seen that multimeric OVA structures are induced by all three treatments. Aggregates are observed that vary in size in all dOVA variants, indicating the presence of cross-beta structure. In nOVA no aggregates are visible on the TEM image.

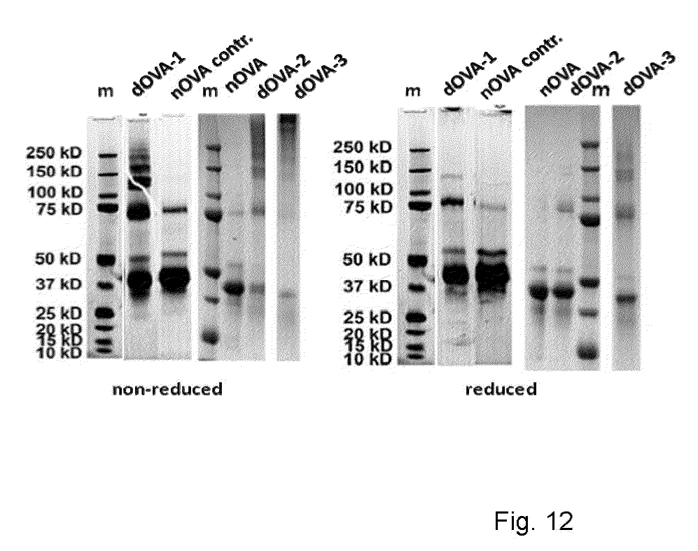

SDS-PAGE analysis of the OVA samples. FIG. 12 shows the analysis of the different OVA samples by SDS-PAGE gel electrophoresis under non-reducing and reducing conditions. The nOVA sample appears as a prominent band at around 40 kDa. A less prominent band is observed at 75 kDa, this band disappears upon reduction. All dOVA forms comprise the same OVA bands as nOVA, albeit in lower or much lower amount depending on the treatment condition used to induce cross-beta structural elements. In addition, high molecular weight bands are seen in all dOVA forms, indicative for the presence of multimers that do not separate under the conditions of SDS-PAGE analysis. dOVA-2 and dOVA-3 display a smear of higher molecular weight bands, these bands run higher in the gel than the high molecular weight bands of dOVA-1. Upon reduction part of the high molecular bands disappear to the 40 kDa band. In conclusion, the various dOVA samples comprise different multimeric properties and more multimers compared to nOVA.

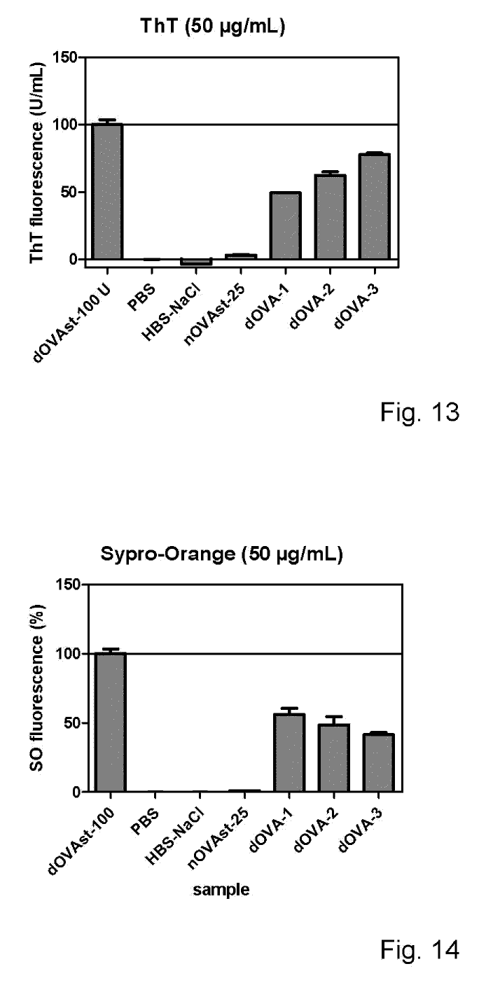

Enhancement of Thioflavin T fluorescence under influence of various OVA forms. Binding of Thioflavin T and subsequent enhancement of its fluorescence intensity upon binding to a protein is a measure for the presence of cross-beta structure which comprises stacked beta sheets. For measuring the enhancement of Thioflavin T fluorescence, OVA samples were tested at 50 μg/ml final dilution. Dilution buffer was PBS. Negative control was PBS, positive control was 100 U/ml standard (reference) misfolded protein solution, i.e., dOVA standard. dOVA standard is obtained by cyclic heating from 30 to 85° C. in increments of 5° C./minute a 1 mg/ml OVA (ovalbumin from chicken egg white Grade VII, A7641-1G, Lot 066K7020, Sigma) solution in PBS. FIG. 13 shows the analysis of OVA samples with ThT. Applying the three outlined cross-beta inducing procedures results in an increase in Thioflavin T fluorescence. The highest increase is seen with dOVA-3; approximately a 25-fold increase when compared to nOVA. dOVA-1 and dOVA-2 are increased 15 and 19 times respectively compared to nOVA (Table 4).

Enhancement of Sypro Orange fluorescence. Sypro Orange is a probe that fluoresces upon binding to misfolded proteins. As a measure for the relative content of proteins comprising cross-beta structure, enhancement of Sypro Orange fluorescence is tested with OVA samples at 50 μg/ml final dilution. Dilution buffer was PBS. Negative control was PBS, positive control was 100 μg/ml dOVA standard. The results are shown in FIG. 14 and Table 5. Applying misfolding results in an increase in Sypro Orange fluorescence. The highest increase is seen with dOVA-1; approximately a 60-fold increase when compared to nOVA. dOVA-2 and dOVA-3 are increased 55 and 45 times respectively. The trend is now opposite from the ThT data.

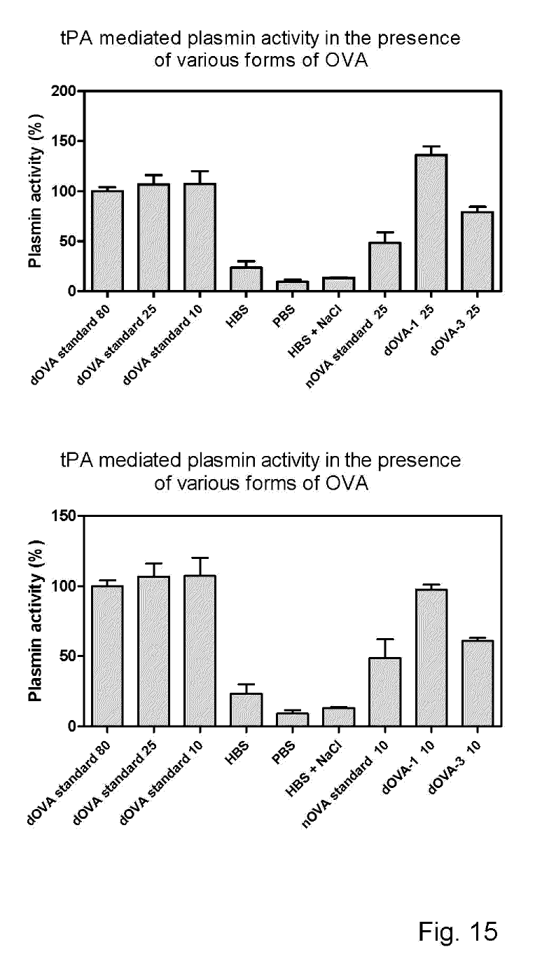

Stimulation of tPA-mediated plasminogen activation by OVA samples. The OVA samples were tested for their tPA mediated plasminogen activation potency at a concentration of 25 and 10 μg/ml. The results are shown in FIG. 15 and Table 6. The activation potency expressed as conversion of plasmin chromogenic substrate is higher for all dOVA forms compared to nOVA upon misfolding and highest for dOVA-1 and dOVA-2 (identical to dOVA standard used as reference in these and other studies).

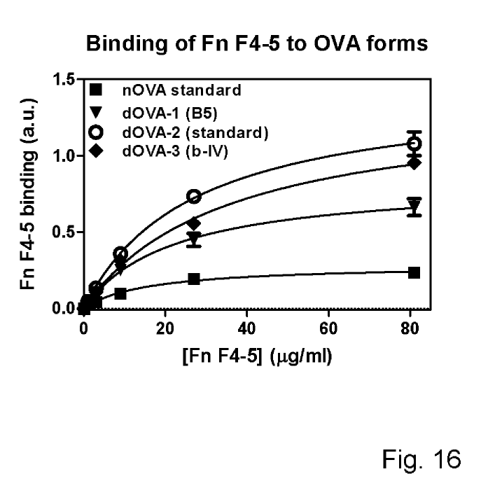

Binding of Fn F4-5 to various forms of OVA, as determined in an ELISA with immobilized forms of OVA. FIG. 16 shows the results of an ELISA to determine the binding of FN4-5 to OVA samples. Table 7 shows the Bmax and kD. Upon misfolding for all samples Bmax is increased up to 5 times (for dOVA-2 and dOVA-3). For dOVA-1 Bmax is increased by a factor of 2. kD does not change much upon misfolding, for samples dOVA-2 and dOVA-3 the kD is increased by a factor of 2. For dOVA-1 the kD value stays the same or is increased by a factor of 1.4. In general one can state that upon misfolding more binding sites for FnF4-5 are created, but the affinity is not changed.

Binding of monoclonal antibodies to various forms of OVA, as determined in an ELISA with immobilized forms of OVA. Tables 8 and 9 show the results (Bmax and kD) of binding analysis by ELISA of several antibodies to nOVA and the dOVA samples.

### T Cell Activation Analysis

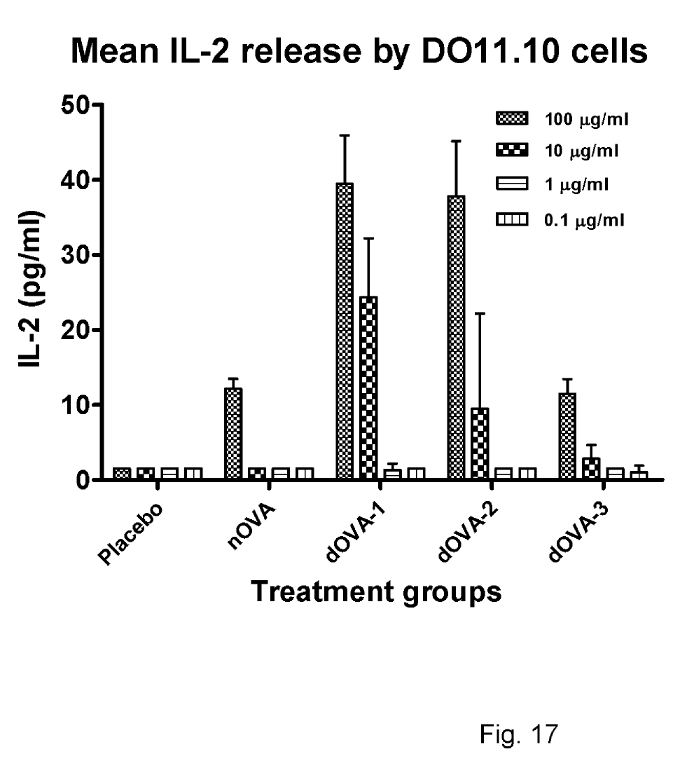

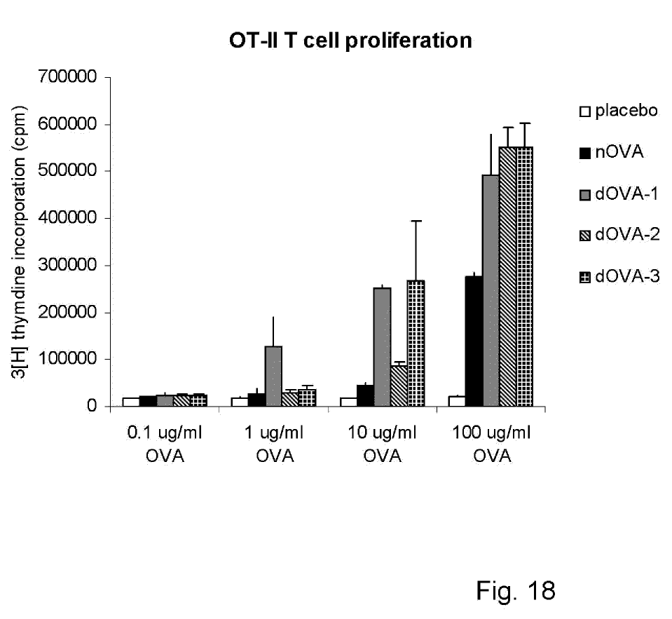

Activation of CD4 T cells, MHCII-Ag presentation. The effect of different structural OVA variants on the efficacy on antigen processing and presentation by dendritic cells (DCs) was tested in vitro. To this end, murine bone marrow derived dendritic cells (BMDC) were pulsed with various concentrations of structurally different OVA samples and co-cultured with OVA specific CD4⁺ T cell line DO11.10 or primary OT-II T cells. Efficient processing and successful presentation of OVA results in T cell activation, as quantified by IL-2 secretion (DO11.10) or proliferation (OT-II) of the T cells. BMDC pulsed with dOVA (100 μg/ml) were more potent in activating DO11.10 T cells, as measured by IL-2 production (FIG. 17) compared to BMDC pulsed with nOVA. dOVA-1 was the most potent, compared to dOVA-3 and dOVA-4 and nOVA in inducing IL-2 expression in DO11.10 cells. Buffer control induced no IL-2 production, whereas DO11.10 specific OVA 323-339 peptide was very efficient in inducing IL-2 production (not shown). When OVA-pulsed BMDC were co-cultured with primary (naïve) T cells (OT-I and OT-II) dOVA1, 2 and 3 were potent inducers of T cell proliferation at a concentration of 1 μg/ml in inducing primary T cell proliferation compared to nOVA (FIG. 18). dOVA-1 and 3 were most efficient. dOVA2 was not capable of inducing more potent OT-II T cell proliferation compared to nOVA at all concentrations tested. Taken together, cross-beta structural variants dOVA2 and dOVA4 were most potent in inducing Ag presentation in the context of MHCII compared to native protein nOVA. In contrast, MHCII presentation of dOVA2 and dOVA3 was as inefficient as nOVA in the activation of DO11.10 T cells, whereas dOVA3 is more potent compared to nOVA in the induction of OT-II proliferation.

### Activation of CD8 T Cells, MHCI-Ag Cross-Presentation

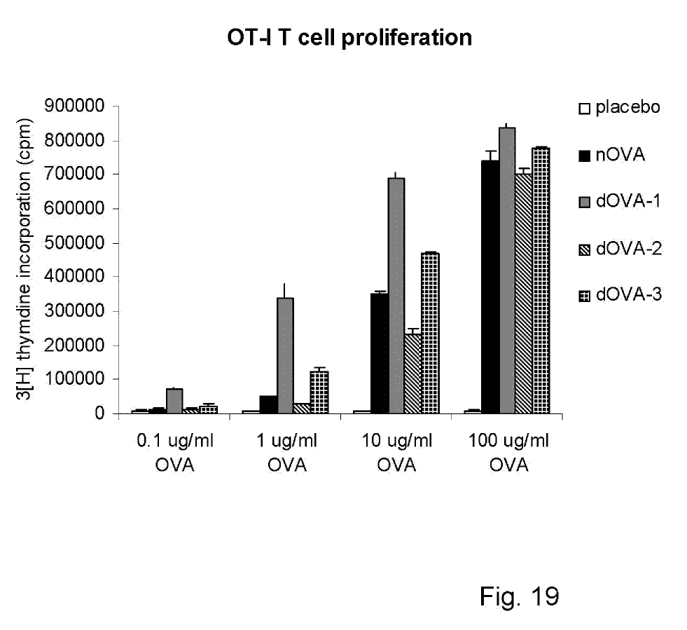

The effect of different structural OVA variants on the efficacy on antigen processing and presentation by dendritic cells (DCs) in the context of MHCI, a process called cross-presentation, was tested. To this end, murine bone marrow derived dendritic cells (BMDC) were pulsed with various concentrations of structurally different OVA samples and co-cultured with primary OVA specific CD8⁺ OT-I T cells. Efficient uptake and cross-presentation of OVA results in induction of proliferation of OT-I T cells. BMDC pulsed with all structurally modified OVAs were more efficient compared to BMDC pulsed with nOVA in induction of proliferation at a concentration of 100 μg/ml (FIG. 19), At lower OVA concentrations, dOVA-1 was the most potent forms of OVA to induce T cell proliferation, while dOVA-2 was the least potent form of dOVA. Buffer control induced no proliferation, whereas OT-I specific OVA 323-339 peptide was very efficient in T cell activation (not shown). Taken together, structural variants dOVA-1 and dOVA-3 were most potent in inducing Ag cross-presentation in the context of MHCI compared to untreated protein nOVA. At high Ag concentrations all dOVA forms were potent compared to nOVA in inducing T cell proliferation, while at low concentrations of OVA, MHCI presentation of dOVA-2 and was the least efficient of the dOVA variants.

### Immune Activating Potential of Structurally Different OVAs In Vivo

Description of study. The immune-activating potential of structurally different OVAs were determined in vivo. Therefore, groups of 13 mice (C57B16) were immunized subcutaneously 4 times with 5 μg OVA/100 μl at weekly intervals. Four and nine days after the last immunization respectively anti-OVA antibody titers and ex vivo T cell activation were determined. Table 10 shows the OVA samples that were used to immunize each different group. Group 1 did not receive an OVA sample, but only buffer (placebo group).

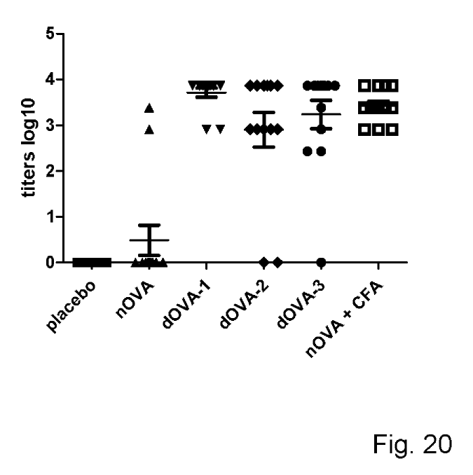

Humoral response. Total anti-OVA IgG/IgM present in the serum on day 25 was highest in the groups immunized with dOVA and comparable to the levels observed after immunization in the presence of complete Freund's adjuvant (CFA, FIG. 20). The highest titers were observed in mice immunized with dOVA-1, even titer higher than 7290 (see Table 11). Taken together, structurally different OVAs, induce IgG and IgM response comparable to those induced by OVA+CFA and are much more efficient in inducing an IgG/IgM response in vivo compared to nOVA.

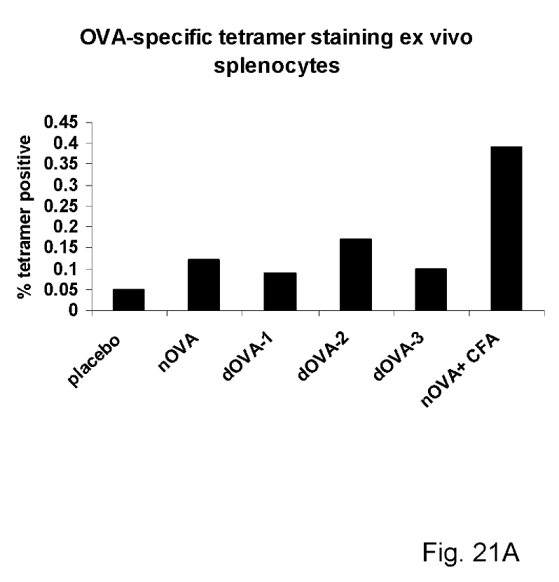

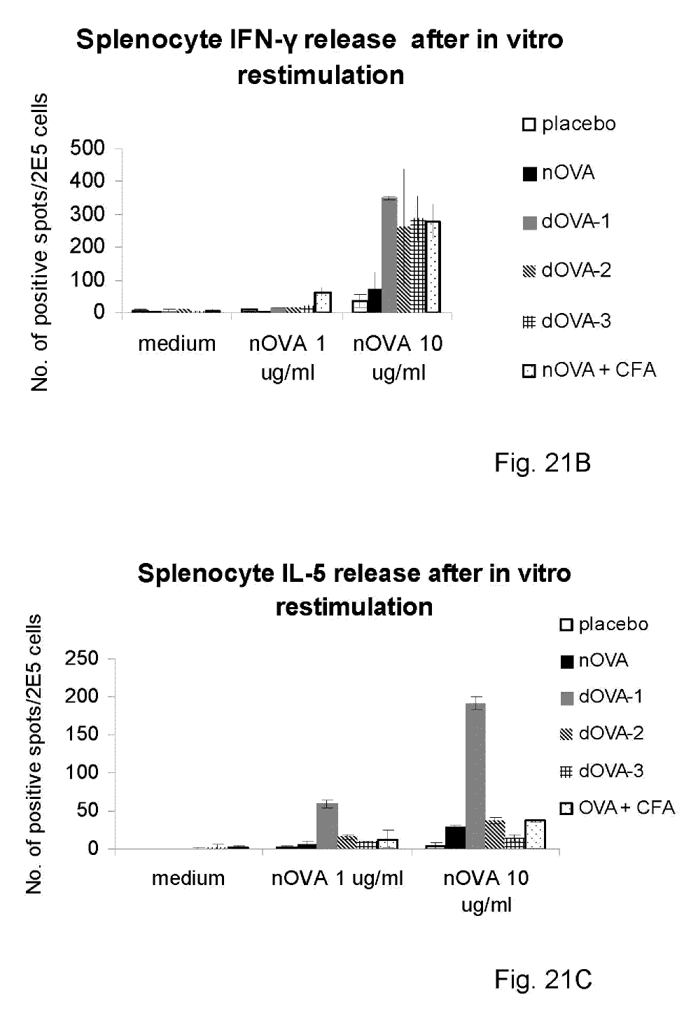

T cell response upon immunization with dOVA variants vs nOVA. Next, the OVA-specific T cell response was determined. Therefore, splenocytes were isolated from three mice (that had a mean antibody titer) of each group and re-stimulated in vitro with nOVA. OVA specific T cells response determined by MHCI tetramer staining, T cell proliferation and IFNγ production (FIG. 21). Splenocytes isolated from mice immunized with nOVA+CFA showed the highest percentage of MHCI-tetramer staining: 0.4% of CD8⁺ T cells were positive (FIG. 21A). Splenocytes isolated from mice immunized with either nOVA, dOVA-2, dOVA-3 and dOVA-4 showed intermediate, but comparable levels of MHCI-tetramer staining. Very clear differences between IFNγ and IL-5 production by OVA specific T cells isolated from immunized mice were observed when re-stimulated in vitro. T cells isolated from dOVA-1, dOVA-2 and dOVA-3 released the highest amount of IFNγ, comparable to levels release by T cells from nOVA+CFA immunized mice. Splenocytes isolated from mice immunized with dOVA-1 were clearly the highest IL-5 producing cells (FIG. 21). Splenocytes isolated from mice immunized with other forms were comparable to nOVA. Taken together, T cells isolated from dOVA-1 immunized mice produced high levels of IFNγ and IL-5 and T cells from dOVA-2, dOVA-3 and nOVA+CFA immunized mice released high IFNγ levels.

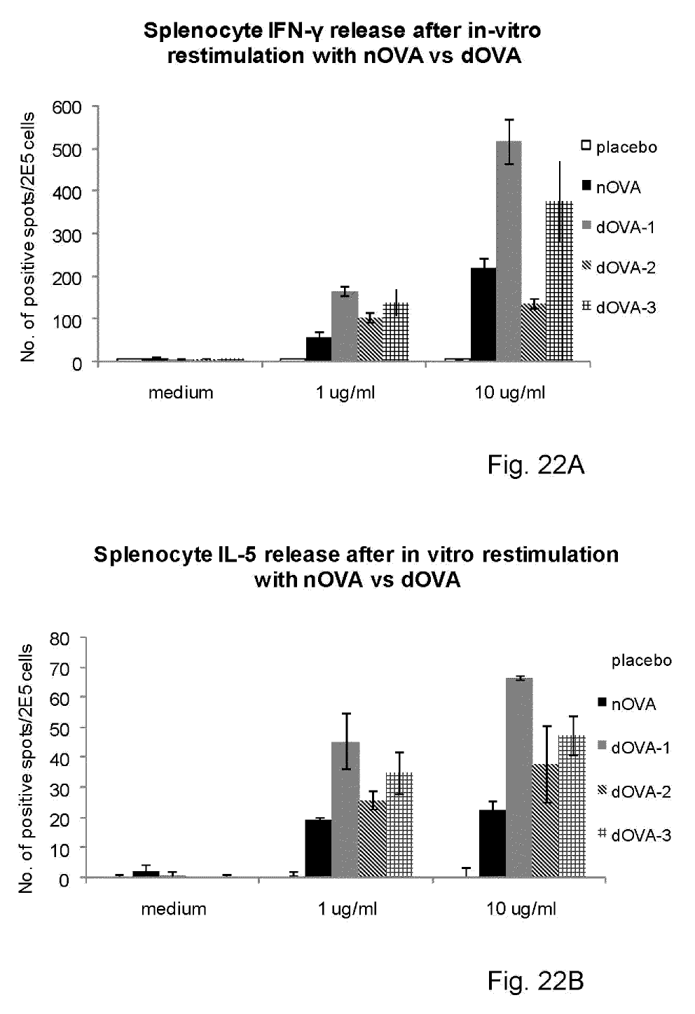

In addition, splenocytes isolated from mice immunized with nOVA+CFA were restimulated ex vivo with nOVA or the structural variants of dOVA. Restimulation with dOVA-1 and dOVA-3 were most potent in T cell activation in terms of IFNγ and IL-5 release (FIG. 22). dOVA-3 restimulation induced comparable levels of IFNγ and IL-5 release to nOVA restimulation.

These results demonstrate that it is possible to select an immunogenic compound comprising a T cell epitope and a cross-beta structure by using either naïve T cells or primed T cells, even a T cell clone with a known T cell epitope, for example here DO11.10, OT-I and OT-II cells, isolated from a mammal, in this case a mouse. Immunogenic compounds can be selected for both CD4 and CD8 specific T cells, for example in this case by using OT-I and OT-OT-II cells.

Tumor Growth in Response to Immunization with OVA Comprising Cross-Beta Structure

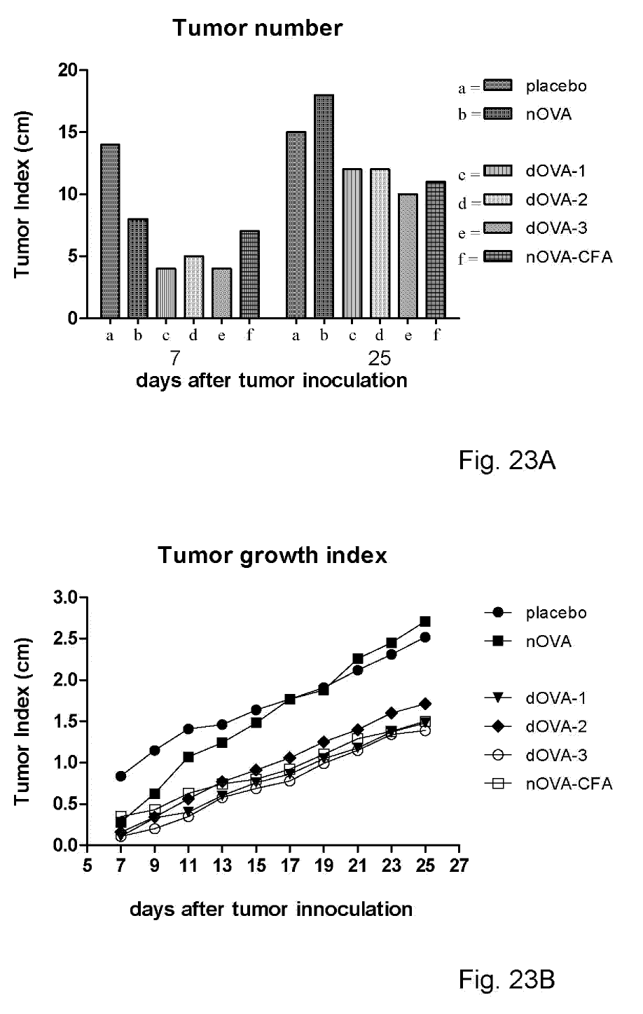

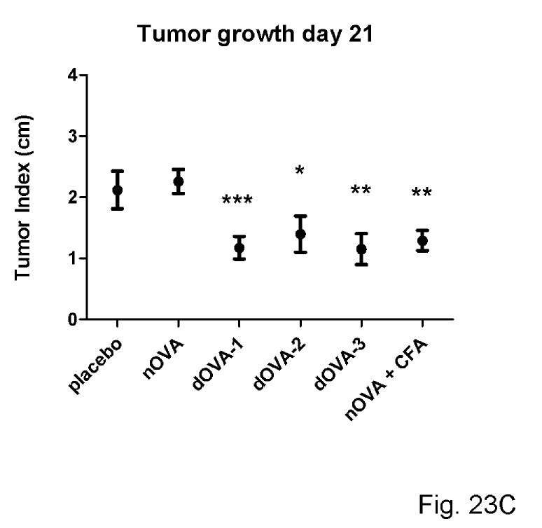

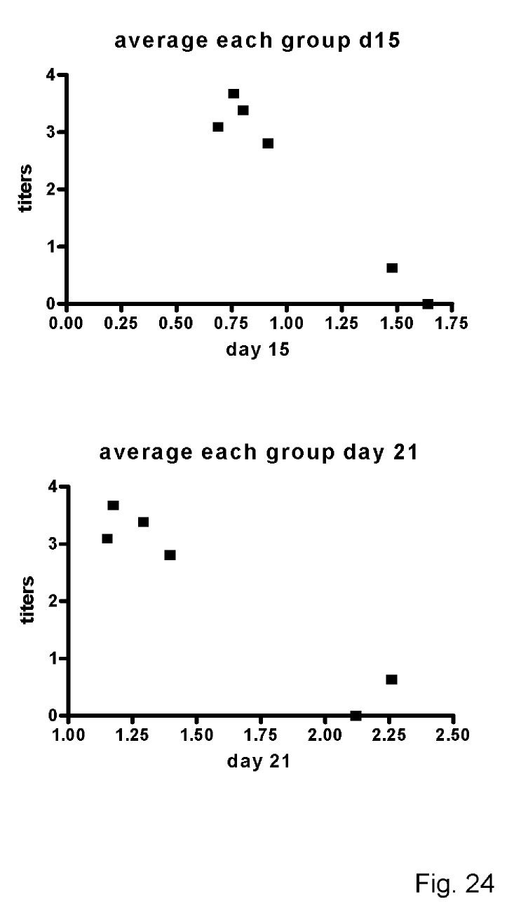

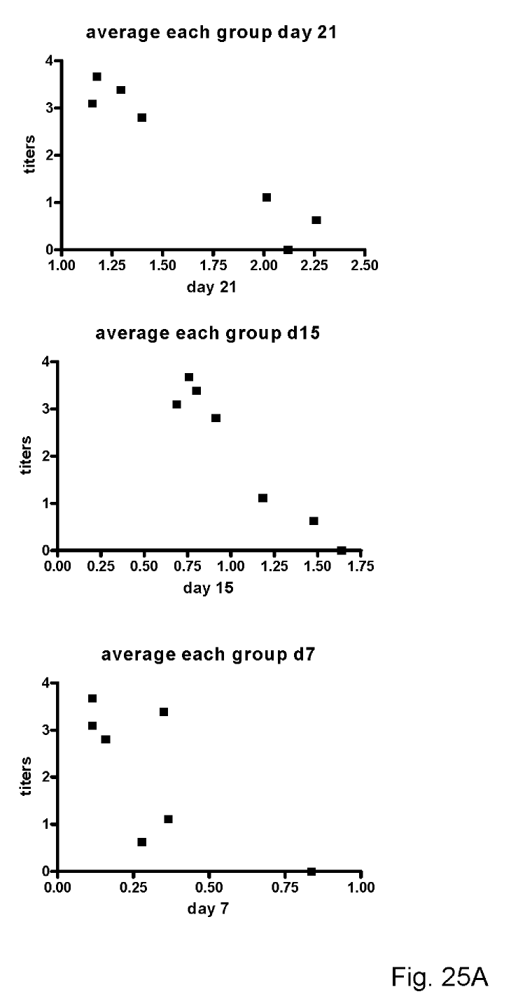

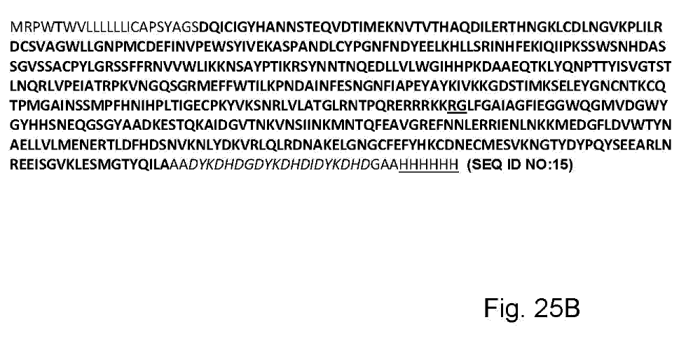

The efficacy of the response to immunization with OVA samples was tested using the growth of EG-7 tumor cells in vivo. Ten mice of each group were inoculated with 5×10e5 tumor cells in the flank on both sides. Tumor take and growth was measured. Tumor take in dOVA-1 (12/20), dOVA-2 (12/20) and dOVA-3 (10/20) immunized mice was decreased compared to placebo (15/20) and nOVA (18/20) immunized mice (FIG. 23). Tumor growth, measured as tumor index was also decreased (FIG. 23). It was observed that tumor growth correlated with increased T cell activation, but also with the antibody titers in each group (FIG. 24, FIG. 25A). Taken together, introduction of different cross-beta structures within OVA can induce a protective immune response in vivo.

These results demonstrate that it is possible to select an immunogenic compound comprising a T cell epitope and a cross-beta structure by using either naïve T cells or primed T cells, even a T cell clone with a known T cell epitope, for example here DO11.10, OT-I and OT-II cells, isolated from a mammal, in this case a mouse that can induce an effective immune response, in this case inhibition of tumor growth.

### Material & Methods

Cell lines. T cell line DO11.10 (CD4⁺, Ia^{d} restricted) was kindly provided by Dr J Leusen (UMC Utrecht) and were propagated in RPMI 1640 (Gibco BRL, Life technologies Paisley UK) supplemented with 10% heat inactivated FBS (Hyclone, Logan, Utah) and 50 IU/ml pencillin (Gibco BRL), and referred to as RPMI⁺ medium. DO11.10 T cell receptor is specific for OVA₃₂₃₋₃₃₉ ISQAVHAAHAEINEAGR in the context of MHC class II. EL-4 (TIB-39, ATCC) and E.G7-OVA (CRL-2113, ATCC) were cultured in RPMI⁺ supplemented with 0.05 mM 2-mercaptoethanol and 0.4 mg/ml G418 (Roche diagnostics) for E.G7-OVA.

Mice. Ten to twelve week old C57BL/6 and Balb/C mice were obtained from Harlan (Horst, NL). OT-I Tg (TcraTcrb) 1100Mjb/J and OT-II Tg (TcraTcrb) 425Cbn OVA-transgenic mice were kindly provided by Dr K. Tesselaar (UMC Utrecht, NL). All animal experiments were performed in compliance with institutional guidelines of AALAC (Association for Assessment of Laboratory Animal Care) and were approved by the institutional animal care and ethics committee.

T cell isolation. CD4⁺ T cells or CD8⁺ T cells were purified from peripheral lymph nodes from OT-II and OT-I mice respectively by positive selection with either αCD4 or αCD8 magnetic MACS beads (Miltenyi Biotec). Populations were reproducibly >98% pure.

Generation of DCs. Murine dendritic cells were cultured from bone marrow as described (Inaba et alj exp med 176: 1693). Briefly, bone marrow cells were isolated from either Balb/C of C57BL/6 murine femurs, and cultured at 1×10⁶ cells per ml RPMI 1640 medium containing 10% FBS 501 U/ml pencillin (RPMI⁺) in the presence of 10 ng/ml GM-CSF (PMC2016, Bioscource). At day 7 DCs (DC7) differentiation and maturation state was confirmed by cell surface expression of CD11c⁺/CD11b⁺ and CD86^{lo}/CD32/16^{hi} and MHCII^{lo} expression respectively. Therefore, DC were stained with a panel of fluorochrome-conjugated Abs as indicated, all purchased at PharMingen (PharMingen SanDiego, Calif.). Non-specific FcR binding was prevented with FcR blocking Ab, clone 2.4G2 (Pharmingen). Fluorochrome labeled isotype controls were used as negative controls. Stained cells were analyzed by flow cytometry using a FACScalibur (BD Bioscience).

MHCI-II (cross) presentation. Ag processing and presentation in the context of MHCI and MHCII was assayed in vitro by pulsing murine bone marrow derived dendritic cells with ovalbumin and subsequent co-cultured with T cells. Therefore, DC7 cells were washed twice with RPMI⁺ medium supplemented with GMCSF and seeded in 96 well round bottom plates at a concentration of 0.5×10E6 cells/ml or 1×10E6 cells/ml. DCs were pulsed with the indicated structurally different OVAs at a concentration of 0.1-1-10-100 μg/ml in a total volume of 200 μl RPMI+GMCSF. Excess OVA (400 μg/ml), and SIINFEKLL/OVA 323-339 (124 μg/ml) were used as positive controls. After 24 hours, pulsed DCs were washed twice with RPMI⁺ medium and co-cultured with 1×10⁵ RF33.70, OT-I and OT-II T cells (DCs derived from C57BL/7), or with DO11.10 (DCs derived from Balb/C). Supernatant were harvested from T cell lines after 24 hours at 37° C. and stored at −20° C. until further analysis. Proliferation of OT-I and OT-II T cells was assayed after 48 hours and 72 hours incubation at 37° C. by ³[H]-thymidine incorporation.

IL-2 ELISA. Secretion of interleukin 2 (IL-2) by RF33.70 and DO11.10 co-cultured with OVA-pulsed dendritic cells (DC) was determined by ELISA (Beckton Dickinson optEIA IL-2 ELISA catnr 555148). Therefore, 50 μl supernatant was collected from T cell-DC cultures after 24 hours and stored in −20 C until further analysis. Levels of IL-2 were determined as described by the manufactures protocol. In short, 96-well plates (Greiner hi-bond catnr 655092) were coated overnight at 4° C. with anti-IL-2 capture antibody 1/250 diluted in 0.1 M sodium carbonate buffer in a total volume of 50 ul. The wells were washed 5 times with PBS-0.05% Tween, followed by blocking with 200 μl PBS-10% FBS for 1 hour at room temperature (RT). After a second sequence of washing, wells were incubated with 50 μl of undiluted collected supernatant and 50 μl recombinant IL-2 standard diluted in PBS-10% FBS (at 200-100-50-25-12.5-6.25-3.125 pg/ml) for 1 hour at RT. Subsequently, the wells were washed 5 times and incubated for 1 hour at RT with anti-IL-2-biotinylated antibody and streptavidin-horseradish peroxidase both diluted 1/250 in PBS-10% FBS. After 10 washes with PBS-0.05% Tween 100 μl TMB substrate solution was added to each well and incubated 5 minutes in the dark. The reaction was stopped with 50 μl 2 M H₂SO₄ per well and absorbance was measured at 450 nm.

Immunization of mice & tumor challenge. Ten- to 12-week-old C57BL/6 mice were immunized subcutaneously on days 0, 7, 14 and 21 with 5 μg of OVA or structural derivatives of OVA in 100 μl PBS. Injection of PBS only was used in the placebo group. In each group, 10 mice were used. At day 25, blood was drawn from the mice and serum was collected by centrifugation and analysed for total IgG. Three mice from each group were sacrificed for ex vivo T cell analysis. Therefore, splenocytes were isolated and single cell suspensions were prepared. The remaining seven mice were challenged on day 28 by injection of 5×10⁵ EG.7-OVA cells in each flank intradermally in a volume of 100 μl. Tumor size was measured on day 7, 9, 11, 13, 15, 17, 19, 21, 23 and 25 after tumor inoculation. The tumor index was determined by (a*b)^{0.5} in which a is the longest diameter and b the shortest diameter of the tumors.

IgG/IgM ELISA. Antibody titers were determined for each individual serum against OVA using enzyme-linked immunosorbent assay (ELISA). Briefly, OVA was coated on 96-well plates (655092, Greiner Microlon) at a concentration of 1 μg/ml in 0.1 M Sodium Carbonate, PH9.5. All incubations were performed for one hour at room temperature (RT) intermitted with five repeated washes with PBS/0.1% Tween. The wells were blocked with 200 μL of blocking buffer (Roche Block) washed and subsequently incubated with dilutions of the sera. As positive controls, monoclonal anti-OVA IgG (A6075, Sigma) was included in each plate. Total IgG/IgM was determined using rabbit-anti-mouse peroxidase labeled-conjugate (P0260, DakoCytomation) followed by incubation with TMB substrate (tebu Bio laboratories). Reaction was stopped using 2 M H₂SO₄. Final titers were determined after subtraction of the no-coat controls. The titer was determined as the reciprocal of the dilution factor that resulted in a signal above the mean signal plus 2 times the standard deviation of the placebo group.

Ex vivo tetramer staining. OVA-specific T cells were analyzed using MHCII tetramer staining. 2×10⁶ splenocytes isolated from immunized mice were washed in PBS-0.5% BSA and were stained with 10 μl of H2-Kb/OVA APC tetramers (M2711, Sanquin). After 15 minutes incubation at RT, cells were stained with PercP conjugated CD8 (clone 53-6.7, Beckton Dickinson 553036) for another 20 minutes at 4° C. After two washed cells were analyzed by flowcytometry using FACS Caliber (Beckton Dickinson).

IFNγ/IL-5 ELISPOT. Production of IL-5 and IFNγ for ex vivo restimulated splenocytes was measured with IL-5 and IFNγ EliSPOT assay (U-Cytech, cat #CT317-PR5) according to manufacturers protocol. Briefly, flat-bottomed polyvinylidene difluoride-supported 96-well culture plate (MilliPore, cat #MSIPS4510 were coated with either anti-IL-5 or anti-IFNγ capture antibody. After 48 hours, plates were washed five times with PBS-0.05% Tween and blocked with manufactures blocking buffer for 1 hour at RT. After five washes, single cell suspensions of splenocytes were restimulated with OVA whole protein the at the indicated concentration, or with peptide (OVA₂₅₇₋₂₆₄ SIINFEKLL or OVA₃₂₃₋₃₃₉ ISQAVHAAHAEINEAGR, purchased at Ansynth) at a concentration of 10 μg/ml. In addition, anti-CD28 and anti-CD28 Abs (BD Pharmingen, cat #553294) anti-CD49d Abs (B&D Biosciences, cat #553313) were added to the cultures at 2 μg/ml. As positive controls, PMA-ionomycin was added to the cultures at 100 ng/ml and 1 μg/ml respectively and staphylococcal enterotoxin B (SEB, Sigma catnr S-4881) at 10 μg/ml. After 48 hours, plates were washed five times with PBS-0.05% Tween and incubated for one hour at 37° C. with detection anti-IFN-γ Ab conjugated to biotin followed by streptavidin-peroxidase (both diluted 1:100 in manufactures dilution buffer). Spots were visulaised by AEC chromogen solution and counted by automatic spot reader (AELVIS ELISPOT microplate reader).

Proliferation of T cells. Proliferation of OVA-specific T cells was measured by ³[H] thymidine incorporation. Splenocytes were restimulated in vitro in 96-well flat bottom plates (167008, Nunc) with the indicated amount of OVA or peptide (OVA₂₅₇₋₂₆₄ SIINFEKLL or OVA₃₂₃₋₃₃₉ ISQAVHAAHAEINEAGR, purchased at Ansynth) at a concentration of 10 μg/ml. As positive controls, PMA-ionomycin was added to the cultures at 100 ng/ml and 1 μg/ml respectively and staphylococcal enterotoxin B (SEB, Sigma catnr S-4881) at 10 μg/ml. After 48 and 72 hours supernatant was collected and stored at −20° C. until further analysis of IFNγ secretion and 1 μCu of ³[H] thymidine was added to the cultures. Cells were harvested and ³[H] thymidine incorporation was measured.

IFN-γ ELISA. Secretion of IFNγ by splenocytes was determined by ELISA (Beckton Dickinson optEIA IFNγ ELISA catnr 555138). Therefore, 100 μl supernatant was collected from splenocyte cultures after 48 and 72 hours and stored in −20° C. until further analysis. Levels of IFNγ were determined as described by the manufactures protocol. In short, 96-well plates (Greiner hi-bond catnr 655092) were coated overnight at 4° C. with anti-IFNγ capture antibody 1/250 diluted in 0.1 M NaCO3 coating buffer in a total volume of 50 μl. The wells were washed 5 times with PBS-0.05% Tween, followed by blocking with 200 μl PBS-10% FBS for 1 hour at room temperature (RT). After a second sequence of washing, wells were incubated with 50 μl of undiluted collected supernatant and 50 μl recombinant IFNγ standard diluted in PBS-10% FBS (at 200-100-50-25-12.5-6.25-3.125 pg/ml) for 1 hour at RT. Subsequently, the wells were washed 5 times and incubated for 1 hour at RT with anti-IL-2-biotinylated antibody and streptavidin-horseradish peroxidase both diluted 1/250 in PBS-10% FBS. After 10 washes with PBS-0.05% Tween 100 μl TMB substrate solution was added to each well and incubated 5 minutes in the dark. The reaction was stopped with 50 μl 2 M H₂SO₄ per well and absorbance was measured at 450 nm.

Induction of T Cell Response after Vaccination with an H5 Subunit Vaccine Comprising Cross-Beta Structure

This example demonstrates that proteins comprising cross-beta structure can contribute to the induction of a T cell response. In this example the hemagglutinin protein H5 of influenza H5N1 strain A/HK/156/97 was prepared, the presence and nature of cross-beta structures was analyzed, and the H5 was used for immunization of mice and the T cell response was examined. After purification the protein as isolated comprised proteins with cross-beta structure, and induced a T cell response. In combination with cross-beta comprising forms of ovalbumin (OVA) and bovine serum albumin (BSA), both combinations comprising cross-beta structure, the T cell response was enhanced. Thus this example further demonstrates that an immunogenic composition comprising cross-beta structure and comprising a T cell epitope, even when the exact sequence of the epitope is not yet known, can be prepared by methods according to this invention.

Study Design. Three groups of mice (n=5) were immunized twice at three weekly interval, i.e., at day 1 and 21. Group A was immunized with 12.5 μg H5, prepared as described below. Group B was immunized with H5 in combination with cross-beta structure comprising ovalbumin (Ova; dOVA) and cross-beta structure comprising cross-beta structure bovine serum albumin (BSA; dBSA), prepared as described below. Group C was immunized with buffer (placebo). Ten days after the last immunization the mice were sacrificed for analysis of immune response.

Methods for Preparing H5 and H5 with OVA and BSA with Cross-Beta Structure

Hemagglutinin 5 protein (H5) of H5N1 virus strain A/Hong kong/156/97 (A/HK/156/97) is expressed in 293 cells with a C-terminal FLAG tag and His tag (FIG. 25B for sequence information), and purified using Ni²⁺-based affinity chromatography as described in patent application WO/2007/008070 and further described elsewhere in this application. In this example purified H5 according to this procedure is termed nH5, or non-treated H5. cbH5 used in this example contains 5 μg nH5, 5 μg H5-dOVA and 2.5 μg of H5-dBSA. H5-dOVA was prepared as described in patent application WO/2007/008070. H5-dBSA was prepared as follows. hdBSA was made as a mixture (1:1) of two cross-beta structure comprising forms of BSA, dBSA-I and dBSA-III. dBSA-I and dBSA-III were prepared using non-treated BSA (nBSA, Fraction V, 9048-46-8, ICN biomedicalsm CatNo 160069) dissolved at 1 mg/ml in PBS and put on a roller device for 10 min. at room temperature and subsequently in a water bath at 37° C. for 10 minutes. Endotoxin level was 0.966 EU/10 μg. dBSA-I was prepared by gradually (0.1° C./sec) heating a 100 μl solution of nBSA added with 5 M NaCl to a final concentration of 171 mM in 200 μl PCR tubes (Roche) in a PTC-200 thermal cycler (MJ Research, Waltham, USA) from 25° C. to 85° C. followed by cooling at 4° C. for 2 min. The heating cycle was repeated twice and the BSA was stored at −20° C. dBSA-III was prepared by adding NaOH from a 5 M stock to adjust the pH of the solution to 12 and subsequently gradually (0.1° C./sec) heating a 100 μl solution in 200 μl PCR tubes in a thermal cycler from 25° C. to 85° C. followed by cooling at 4° C. for 2 min. The heating cycle was repeated twice. The pH was adjusted to pH 7 by addition of HCl from a 15% stock solution. The samples were aliquoted and stored at −20° C. H5-dBSA was made by mixing H5 (40 μg/ml and hdBSA (385.8 μg/ml). MES buffer (Sigma, M8250) was added (final concentration 0.02% NaN₃, 0.1 M MES, 0.15 M NaCl, pH 4.7) and EDC to a concentration of 43 mM (Pierce 22980). After resuspension NHS (Pierce, 24500) was added to a concentration of 7.9 mM. The reaction was allowed to proceed for 2 hours at room temp. Subsequently the reaction mixture was dialyzed three times in a Slide-A-Lyzer dialysis cassette (Mw. cut off 10,000, Pierce) at 4° C. for at least 4 hours against PBS. Glassware was cleaned prior to use with NaOH and ethanol. Concentrations of H5 were adjusted to the change in volume due to dialysis.

Endotoxin measurement. The endotoxin level of nH5 is <0.05 EU/ml with 10 μg/ml H5, of H5-dOVA is >4.90 EU/ml with 10 μg/ml H5 and of BSA (source for H5-dBSA) 0.966 EU/ml comprising 10 μg H5/ml. The endotoxin level of the dilution buffer PBS is <0.050 EU/ml.

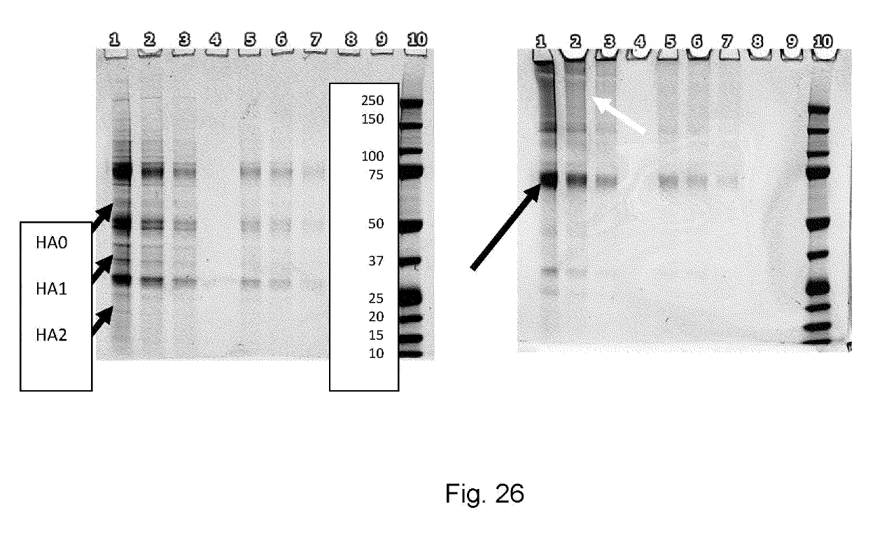

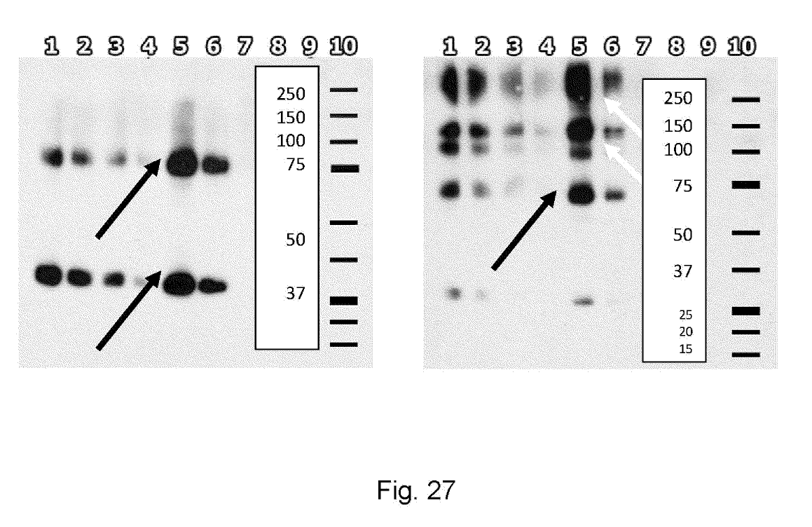

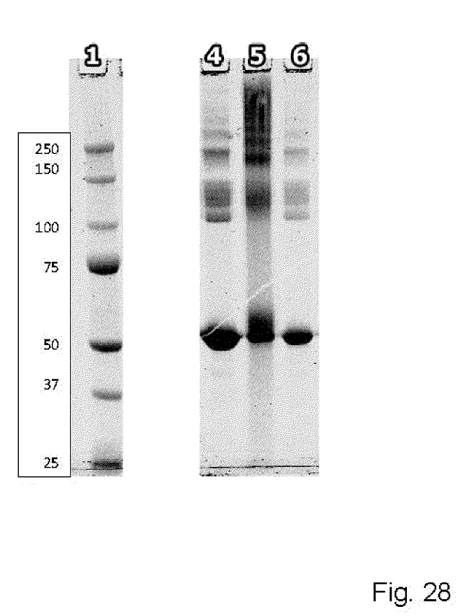

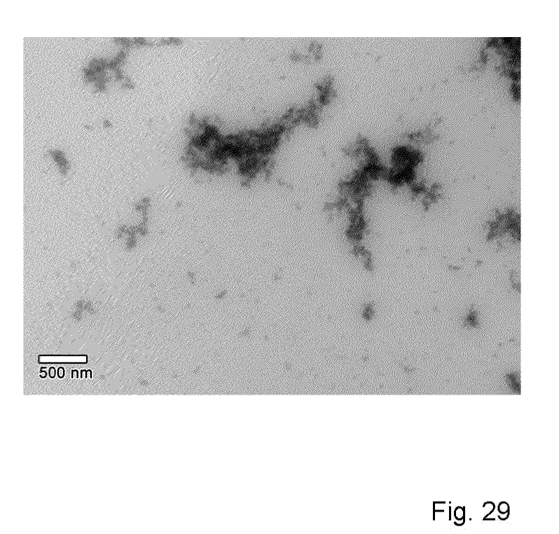

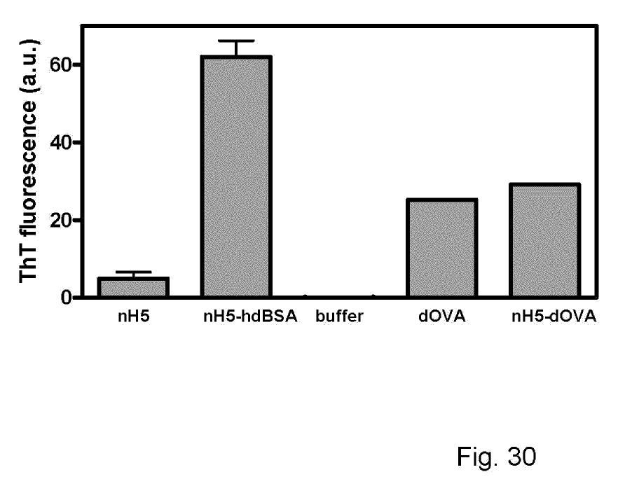

Structural analysis. H5 protein analysis by SDS-PAGE followed by Coomassie staining or Western blot analysis reveals that H5 as purified is partially processed and contains both monomeric and multimeric H5 molecular assemblies (FIGS. 26 and 27). Applying the purified H5 on a size exclusion column revealed that all H5 protein is present as high molecular weight multimers, which are not retained on a Superdex 200 gel filtration column (GE Healthcare). Monomeric and multimeric H5 assemblies seen on gel are therefore the result of sample preparation procedures; in solution only multimers are present. FIG. 28 shows the analysis of hdBSA, demonstrating the induction of multimeric forms of BSA upon misfolding. FIG. 29 shows a TEM analysis of H5-dOVA showing the presence of relatively large amorphous aggregates with dimensions of approximately 250-500 nm*2 μm, and smaller aggregates of approximately 25×25 nm up to approximately 100×100 nm. FIG. 30 shows Thioflavin T fluorescence enhancement analysis, demonstrating an increased signal of dBSA and dOVA samples.

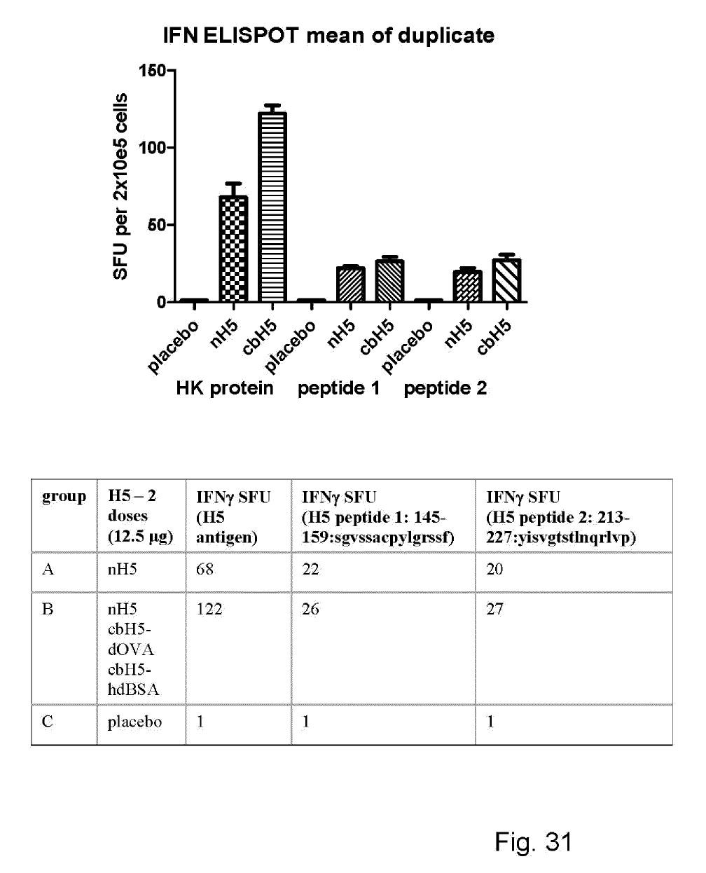

T cell activation analysis. FIG. 31 shows the analysis of T cells isolated from mice immunized with nH5 or nH5 with dBSA and dOVA. T cell activation was measured ex vivo using splenocytes from immunized mice, 10 days after the final immunization, and determining the capacity to induce IFNγ secretion upon incubation with nH5 protein. Activation was measured by ELISPOT method.

### T Cell Activation by Antigen (H5) Comprising a T Cell Epitope and at Least One Cross-Beta Structure

With this example it is demonstrated that the combination of certain cross-beta structures in H5 protein and a certain amount of T cell epitopes required for inducing a T-cell response in mice.

Methods for Preparing Structural Variants of H5 which Comprise Cross-Beta

Theoretical considerations: estimated size and surface of H5 multimers. The average van der Waals radius of the 20 amino acids is approximately 0.3 nm, or 3 Å. The approximate average volume of an amino acid is 110 Å³. The approximate average surface of an amino acid residue is 28 Å², or 0.28 nm². The approximate average mass of an amino acid residue is 120 Da. From these numbers it is estimated that using the 1.000 kDa MW cut-off filter, at maximum protein assemblies comprising approximately 8500 amino acid residues flow through the filter. This maximum size corresponds to a maximum protein surface on for example a TEM image, of 2400 nm². Assuming a spherical or squaric arrangement of the protein multimer, this corresponds to protein structures with a radius of approximately 27 nm, or 50×50 nm squares, respectively, on TEM images. With H5 appearing on the SEC column and on SDS-PA gel as amongst others, 33 kDa and 75 kDa molecules, multimers of up to 30 or 13 H5 monomers will flow through the 1.000 kDa filter, at maximum. By approximation, on average, 1 nm² corresponds to 3.6 amino acid residues or 430 Da, and 1 kDa corresponds to 2.3 nm².

With this approximate numbers it is possible to calculate the number of H5 monomers that appear in multimers, as seen for example under the direct light microscope, in SEC fractions, on TEM images and on SDS-PA gels. These considerations also apply for any other molecular assembly of one or more protein molecules, like for example ovalbumin, E2 and factor VIII.

Endotoxin measurement. The endotoxin content of H5 as supplied by Protein Sciences was measured at 25 μg/ml (diluted in sterile PBS), the concentration of H5 at which vaccination will occur. The Endosafe cartridge had a sensitivity of 5-0.05 EU/ml (Sanbio, NL).

The endotoxin level is 0.152 EU/ml. The endotoxin level of the dilution buffer PBS is <0.050 EU/ml.

Recombinantly produced heamagglutinin 5 (H5) protein of H5N1 strain A/Vietnam/1203/04 (A/VN/1203/04) was purchased from Protein Sciences. The stock concentration was 1 mg/ml (determined with the BCA method (Pierce)) in 10 mM sodium phosphate, pH 7.1, 171 mM NaCl, 0.005% Tween20. H5 is stored at 4° C. The H5 stock as supplied is referred to as cross-beta H5-0, or dH5-0, i.e., H5 that comprises cross-beta structure of arbitrarily chosen type 0. Handlings with H5 solutions are performed under sterile conditions in a flow cabinet. When dH5-0 is ultracentrifuged for 1 h at 100,000*g (4° C.), 62% of the H5 remains in the supernatant; 38% is pelleted. Therefore, 62% of the dH5-0 is designated as soluble H5, 38% as insoluble protein.

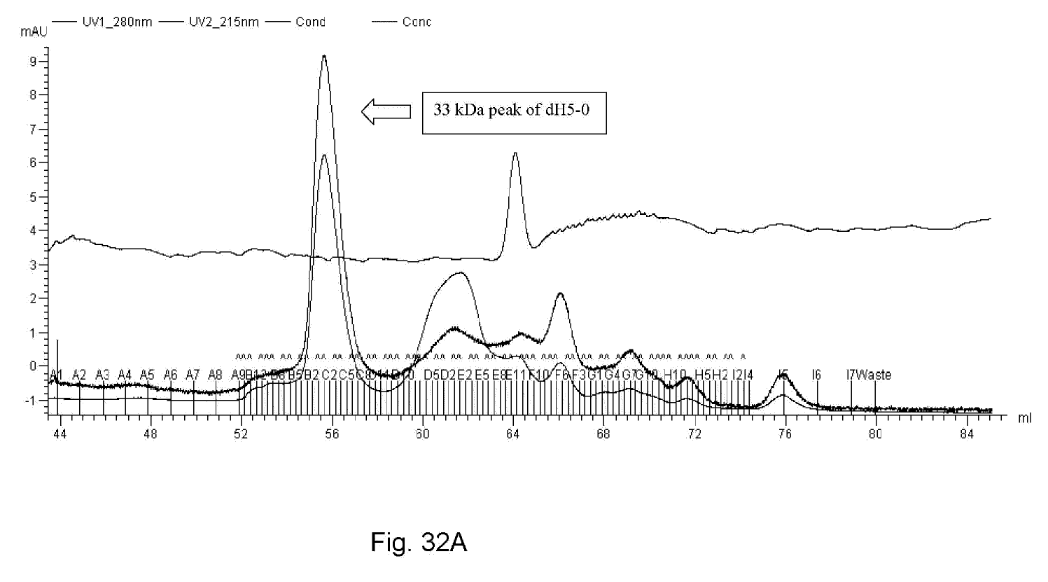

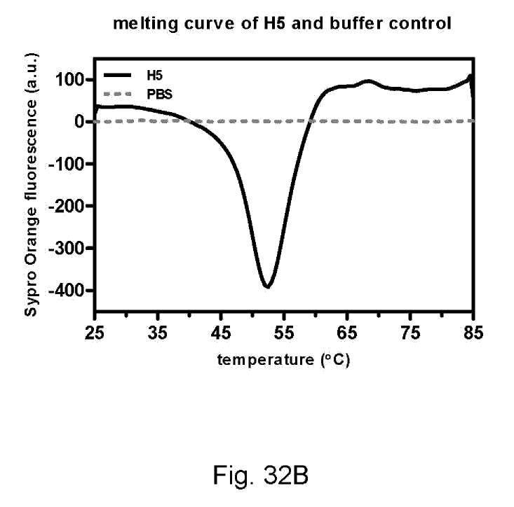

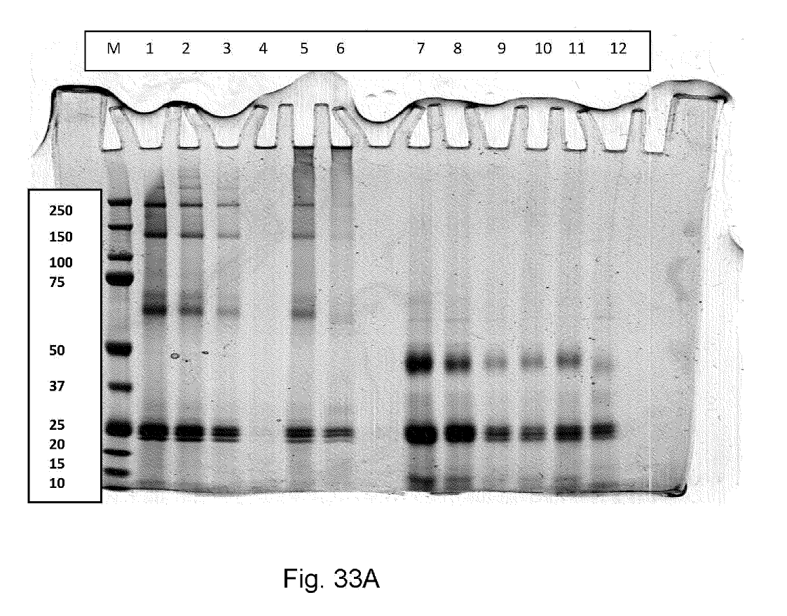

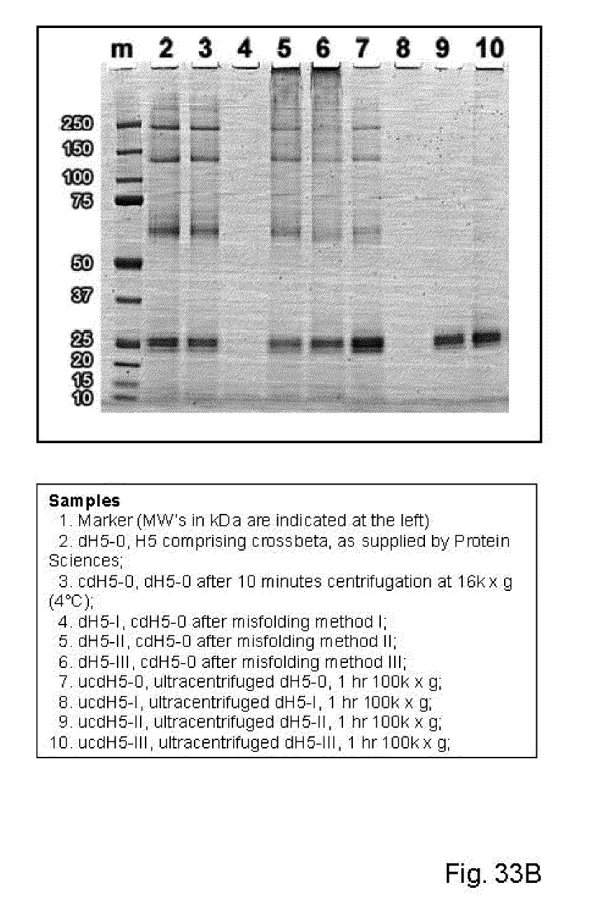

The dH5-0 protein solution is analyzed as supplied and in addition after applying a routine centrifugation step, i.e., 10 minutes centrifugation at 16,000-18,000*g, at 4° C., in a rotor with fixed angle. The dH5-0 after this standard centrifugation step is referred to as cdH5-O, cross-beta H5 after centrifugation. For analysis and vaccination trials, the supernatant of cdH5-0 is used. After the centrifugation run a white pellet becomes visible, indicative for the present of insoluble H5 aggregates. An aliquot of 175 μl of the dH5-0 is subjected to size exclusion chromatography on an analytical superdex75 10/30 column (GE Healthcare) by Roland Romijn (U-ProteinExpress, Utrecht, NL), using an Äkta explorer (GE Healthcare). In FIG. 32A it is seen that one main peak is retained by the SEC column. Calculation of the molecular weight, based on a known calibration curve of the column, revealed that 65% of the loaded dH5-0 eluted as a 33 kDa protein. The remaining protein fraction eluted as proteins with molecular masses of 4 kDa or smaller. Noteworthy, on SDS-PA gel with non-reducing conditions, the eluted 33 kDa dH5-0 fraction appeared with the same protein band pattern as the dH5-0 starting material (See FIG. 33A for dH5-0). Under reducing conditions, both dH5-0 starting material and the 33 kDa dH5-0 fraction appear as two bands of approximately 24 and 48 kDa. Either the four bands with MWs>50 kDa are co-eluted with the main 33 kDa dH5-0 band and are visualized on gel, or dH5-0 stably aggregates after the SEC run into multimers that do not dissociate upon heating in sample buffer with SDS.

Additionally, for several analyses dH5-0 and other H5 samples comprising cross-beta structure are ultracentrifuged for 1 h at 100,000*g, at 4° C., using a rotor with swing-out buckets. The supernatants of these ultracentrifuged H5 samples are used for analyses and are referred to as ucdH5-0 or udH5-0, and ucdH5-I/II/III or udH5-I/II/III.

Ultrafiltrated dH5-0, referred to as fdH5-0, is obtained by filtering cdH5-0 for 10 minutes at 16,000*g through a Vivaspin 500 PrNo VS0161, 1×10⁶ Da MW cut-off filter, at 4° C. The flow-through of the filter is used for subsequent analyses and immunizations, and comprises H5 monomers/oligomers with a molecular weight of approximately <1.000 kDa. The fraction of dH5-0 that is poured through the filter, i.e., fdH5-0, is 80% of the starting material, as determined with the BCA method after three consecutive filtrations. Therefore, the dH5-0 comprises approximately 20% protein multimers with a molecular mass of >1.000 kDa.

Preparation of Misfolded dH5-I Comprising Cross-Beta Structure

dH5-I (heat cycling at pH 7) is produced from dH5-0 supernatant after centrifugation for 10 minutes at 16,000*g (4° C.), i.e., cdH5-0. The H5 concentration is 1 mg/ml. From a 5 M NaCl stock an amount is added to cdH5-0 in order to adjust the NaCl concentration to that of dH5-II (see below). The cdH5-0 is divided in 100 μL aliquots in a 200-μl PCR plate (BioRad, 96 well, cat nr 2239441) and placed in a thermal cycler (Biorad, MyIQ). The cdH5-0 is incubated at 25° C. for 20 seconds and subsequently heated from 25° C. to 85° C., ramp 0.1° C./s, followed by a 20 s incubation at 85° C. This cycle is repeated twice (total cycles is three). The program finishes with cooling at 4° C. for 2 minutes. The dH5-I aliquots are combined and again divided into aliquots in Eppendorf 500 μL cups. Aliquots of 50 μg dH5-I/vial are stored at −20° C.

Before misfolding the protein solution looks clear, after heat denaturation the sample appears white turbid. After freezing-thawing and subsequent centrifugation a pellet is visible. After ultracentrifugation for 1 h at 100,000*g (4° C.), 37% of the H5 remains in the supernatant.

Preparation of Misfolded dH5-II Comprising Cross-Beta Structure

dH5-II (heat cycling at pH 2) is produced from dH5-0 supernatant after centrifugation for 10 minutes at 16,000*g (4° C.), i.e., cdH5-0. The H5 concentration is 1 mg/ml. The pH of cdH5-0 is lowered to pH 2 by addition of HCl from a 15% (v/v) stock in H₂O. Then it is divided into 100 μL per cup in PCR strips (BioRad, 96 well, cat nr 2239441) and placed in a MyIQ RT-PCR cycler (Biorad). The misfolding program is the same as used for preparing dH5-I (see above). Subsequently, dH5-II aliquots are combined and the pH is adjusted back to pH 7 by addition of NaOH solution from a 5 M stock. Then, dH5-II is aliquoted again and stored at −20° C.

Before misfolding the cdH5-0 solution at pH 2 appears clear, after heat denaturation and adjusting the pH back to 7, the dH5-II sample appears slightly turbid. After freezing-thawing and subsequent centrifugation a pellet is visible. After ultracentrifugation for 1 h at 100,000*g (4° C.), 41% of the H5 remains in the supernatant.

Preparation of Misfolded dH5-III Comprising Cross-Beta Structure

dH5-III (prolonged incubation at 5° C. below the melting temperature of dH5-0) is produced from cdH5-0. The H5 concentration is 1 mg/ml. For this, the melting temperature of cdH5-0 at 1 mg/ml was determined using the MyiQ cycler. 0.7 μl Sypro Orange 5000× stock (Sigma) is added to 70 μl cdH5-0 and the sample is heated from 25° C. to 85° C. The ramp rate is set to 0.1° C./min. At each temperature increment of 0.5° C. the Sypro Orange fluorescence is measured at 490 nm (excitation) and 575 nm (emission). The melting temperature was 52.5° C. (See FIG. 21B). Subsequently, cdH5-0 is incubated for approximately 16 h at 47.5° C., i.e., 5° C. below the cdH5-0 melting temperature. Aliquots of dH5-III are then stored at −20° C. Before misfolding the cdH5-0 solution was clear, after prolonged incubation at a temperature of 5° C. below the cdH5-0 melting temperature, the sample is still clear. After freezing-thawing and subsequent centrifugation no pellet is visible. After ultracentrifugation for 1 h at 100,000*g (4° C.), 45% of the H5 remains in the supernatant and is the soluble dH5-III fraction.

Visual inspection of H5 samples before/after various treatments. In Table 1 the results of the visual inspection of the six H5 forms is summarized.

Transmission Electron Microscopy Imaging with H5 Forms with/without Ultracentrifugation

The various H5 forms are subjected to TEM analysis. The dH5-0, dH5-I, dH5-II and dH5-III forms are analyzed directly, and their supernatants after ultracentrifugation for 1 h at 100,000*g (4° C.) are imaged. PBS served as a negative control and gave an empty image, as expected. The dH5-0 appeared with a background of many non-uniformly shaped protein assemblies of approximately 25×25 nm to 100×100 nm, corresponding to molecular H5 assemblies of approximately 270-4300 kDa (approximately 4-57 H5 monomers of 75 kDa). Also large, branched aggregates with strings of protein assemblies are seen. The branches are approximately 100 to 400 nm thick and approximately 2 to 5 μm in length. Upon ultracentrifugation of dH5-0, many string-like protein assemblies are seen, with bead-like subunits. Many have dimensions of approximately 25×50 nm, a few are approximately 100×100 nm up to 400×800 nm. The cdH5-0 appears very similar to udH5-0, with the exception that also larger protein assemblies are seen with dimensions of approximately 1500×1500 nm. The fdH5-0 appears with a background of uniformly shaped relatively tiny protein structures with undefined, though relatively small size and shape. A few relatively large protein structures are seen, which are composed of strings of protein assemblies. These structures have tree-like appearances with branches, and are approximately 400×4000 nm in size. The dH5-I comprises relatively a few but large and dense protein assemblies composed of spherical protein building blocks. The building blocks are connected in branched strings with approximate dimensions of 500×5000 nm. Hardly any H5 is seen in structures apart from the large branched strings. Upon ultracentrifugation, an empty image is obtained, indicated that all dH5-I structures seen before ultracentrifugation are insoluble and pelleted. The dH5-II is seen as amorphous and large protein assemblies with approximate sizes of 3×3 μm. The protein assemblies appear as loosely connected structures. The structures are composed of smaller non-uniformly shaped low-density protein assemblies, which are also seen freely. These building blocks are approximately 50×50 to 100×100 nm in size. Upon ultracentrifugation, the supernatant is fully clear on the TEM image. This shows that H5 multimers are insoluble and pelleted upon ultracentrifugation. The dH5-III is presented on the TEM image as a relatively high number of two types of protein assemblies with a relatively small size of approximately 25×25 nm and approximately 50×50 nm. Upon ultracentrifugation, again many small protein assemblies are seen in the supernatant, on the TEM image. The approximate sizes of the multimers are mostly 20×20 nm with a few protein assemblies of approximately 100×100 nm in size. Apparently, the protein assemblies are soluble and are not pelleted upon ultracentrifugation.

Analysis of H5 forms on SDS-PA gel under reducing and non-reducing conditions. The six H5 structural variants were analyzed on an SDS-PA gel, both with and without a pretreatment in the presence of reducing agent DTT. See FIG. 33A. When comparing the three H5 forms dH5-0, cdH5-0 and fdH5-0 it appears that the number of molecules with a molecular weight of >50 kDa decreases in the order dH5-0>cdH5-0>fdH5-0. It is of note that the protein assemblies that are visible stayed intact after heating for 10 minutes at 100° C. Upon adding DTT during heating, the three H5 forms appear similarly on gel. The dH5-I variant does not enter the gel when non-reducing conditions are applied, indicative for the presence of relatively large multimers that resist heating at 100° C. in the presence of SDS. Upon adding DTT during heating, these multimers dissociate and appear on the gel similarly to the other H5 forms. The dH5-II and dH5-III comprise a relatively high content of multimers with a molecular mass >250 kDa, with large multimers that do not enter the gel, when non-reducing conditions are applied. Under reducing conditions, the H5 forms appear similarly as the other structural variants. These data show that dH5-I comprises relatively the largest multimers, with dH5-II and dH5-III comprising more and higher order multimers than dH5-0 and cdH5-0, and with fdH5-0 comprising least multimers.

SDS-PAGE with H5 samples before/after ultracentrifugation. The dH5-0, dH5-I, dH5-II and dH5-III are subjected to ultracentrifugation for 1 h at 100,000*g (4° C.). This ultracentrifugation is accepted as a procedure for separation of insoluble protein molecules from the soluble fraction that will remain in the supernatant. Together with starting material and cdH5-0, these ultracentrifuged samples are analyzed on an SDS-PA gel. See FIG. 33B. The dH5-0 starting material and cdH5-0 appear in a similar fashion; five protein bands with molecular weights of approximately 25, 60, 140, 240 and 350 kDa. Upon ultracentrifugation of dH5-0, the 25 kDa band becomes more dominant, when the same total amount of H5 is loaded onto the gel (correction factor determined based on BCA protein concentration determination), and when compared to dH5-0 and cdH5-0. The dH5-I sample is not visible on gel at all. Apparently, dH5-I comprises molecular assemblies or multimers that are too large to enter the gel, and that are tightly kept together by relatively strong forces. Interestingly, approximately 37% of the dH5-I stayed in solution upon ultracentrifugation. Apparently, this 37% of the dH5-I molecules is composed of multimers that can not be visualized on the SDS-PA gel. Both dH5-II and dH5-III comprise the same H5 bands as dH5-0 and cdH5-0, when analyzed before ultracentrifugation. In addition, high molecular weight bands are seen in both H5 forms, indicative for the presence of multimers that are tightly kept together. After ultracentrifugation, for both dH5-II and dH5-III all multimer bands and H5 bands with MWs>50 kDa are not seen anymore, indicating that those H5 molecules are pelleted upon ultracentrifugation.

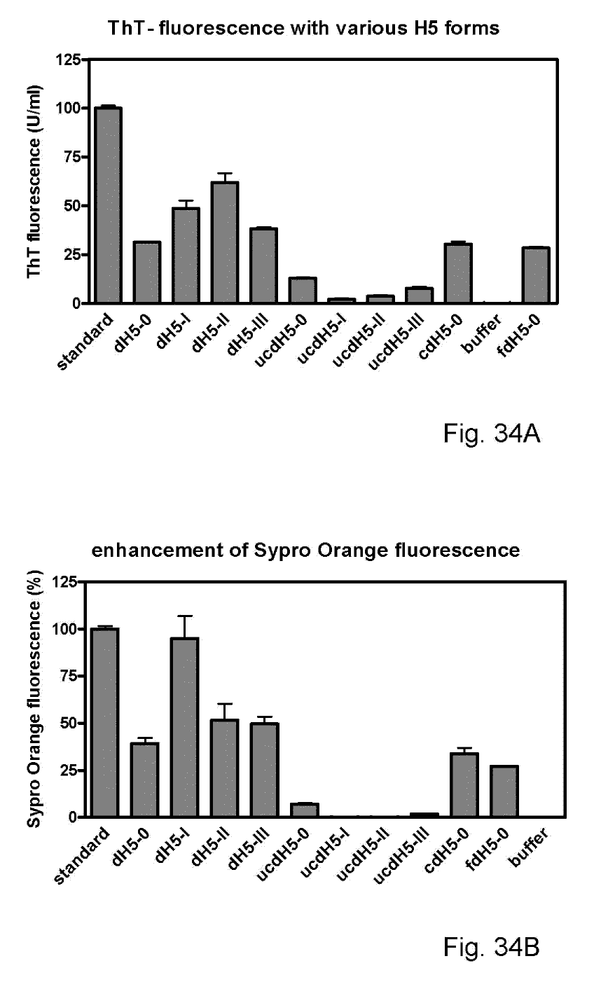

Thioflavin T fluorescence. Binding of Thioflavin T and subsequent enhancement of its fluorescence intensity upon binding to a protein is a measure for the presence of cross-beta structure which comprises stacked beta sheets. For measuring the enhancement of Thioflavin T fluorescence, H5 samples were tested at 100 μg/ml final dilution. Dilution buffer was PBS. Negative control was PBS, positive control was 100 μg/ml standard misfolded protein solution, i.e., dOVA standard. dOVA standard is obtained by cyclic heating from 25 to 85° C. (6° C./minute) of a 1 mg/ml ovalbumin (Albumin from chicken egg white Grade VII, A7641-1G, Lot 066K7020, Sigma) solution in PBS. The H5 samples cdH5-0, dH5-I, dH5-II and dH5-III are also tested after 1 h centrifugation at 100,000*g, at 4° C. Supernatant is analyzed for its protein concentration using the BCA method. Subsequently, adjusted volumes in order to test identical protein concentrations, are used in the Thioflavin T fluorescence enhancement assay. Ultracentrifuged samples are indicated with a “u.” See FIG. 34A for the data. The dH5-0, cdH5-0 and fdH5-0 display very similar fluorescence enhancement, indicative for the presence of cross-beta structure to a similar extent. Applying misfolding protocols I-III results in an increase in Thioflavin T fluorescence, and therefore an increase in cross-beta content. The highest increase is seen with dH5-II; approximately a twofold increase when compared to dH5-0. For cdH5-0 approximately 50% of the fluorescence signal remains in the supernatant after ultracentrifugation. For ucdH5-I, II, III, most of the Thioflavin T fluorescence enhancing capacity is pelleted upon ultracentrifugation, showing that most H5 molecules with cross-beta structure are assembled in insoluble multimers.

Enhancement of Sypro Orange fluorescence. Sypro Orange is a probe that fluoresces upon binding to misfolded proteins. As a measure for the relative content of misfolded proteins, enhancement of Sypro Orange fluorescence is tested with H5 samples at 25 μg/ml final dilution. Dilution buffer was PBS. Negative control was PBS, positive control was 100 μg/ml dOVA standard. The H5 samples cdH5-0, dH5-I, dH5-II and dH5-III are also tested after 1 h centrifugation at 100,000*g, at 4° C. Supernatant is analyzed for its protein concentration using the BCA method. Subsequently, adjusted volumes in order to test identical protein concentrations, are used in the Sypro Orange fluorescence enhancement assay. Ultracentrifuged samples are indicated with a “u.” See FIG. 34B for the data. The cdH5-0 and fdH5-0 samples display a somewhat lower fluorescence enhancement than their starting material dH5-0. This indicates that after centrifugation for 10 minutes at 16,000*g a fraction of misfolded dH5-0 is pelleted, and that after filtration a fraction of dH5-0 with a molecular weight of >1.000 kDa is retained by the filter and has misfolded protein characteristics. Applying misfolding protocols I-III results in an increase in Sypro Orange fluorescence, that is most pronounced for dH5-I. Compared to the starting material, the Sypro Orange fluorescence is about doubled. For cdH5-0 approximately 25% of the fluorescence signal remains in the supernatant after ultracentrifugation. For ucdH5-I, II, III, most if not all of the Sypro Orange fluorescence enhancing capacity is pelleted upon ultracentrifugation. As seen in the Thioflavin T fluorescence measurement (See FIG. 34A), the supernatant of dH5-III comprises relatively the most misfolded protein, compared to dH5-I and dH5-II.

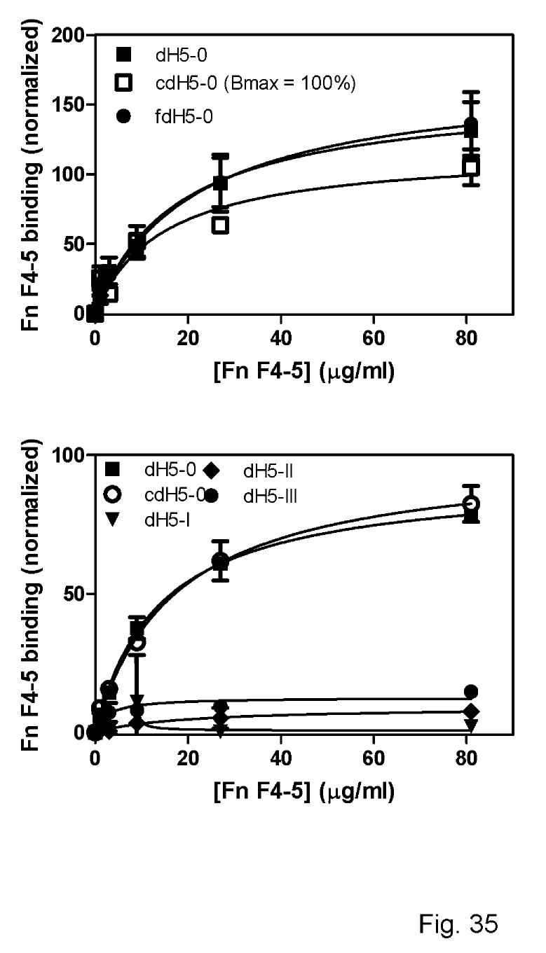

Binding of fibronectin finger 4-5 to H5 forms comprising cross-beta structure. Finger domains of tPA, factor XII, hepatocyte growth factor activator and fibronectin bind to cross-beta structure in protein, when the free finger domains are contacted with proteins comprising cross-beta structure, as well as when the finger domains are part of the full-length or truncated proteins. We now assessed the binding of the fourth and fifth finger domain of fibronectin (Fn F4-5) to the various H5 forms, as depicted in FIG. 35 and Table 13. It is clear that the cross-beta H5 forms dH5-0, cdH5-0 and fdH5-0 bind Fn F4-5 to a far more extent than the dH5-I, dH5-II and dH5-III. Apparently, the increase in ThT fluorescence and Sypro orange fluorescence with these latter three forms, indicative for increased misfolding of the H5 upon the artificial exposure to denaturing conditions as described, is accompanied by a loss in the exposure of binding sites for the natural sensors of cross-beta structure, i.e., the finger domains. This shows that the nature of the cross-beta structure in terms of the molecular assembly, differs between dH5-0, cdH5-0 and fdH5-0 when compared to dH5-I, dH5-II and dH5-III.

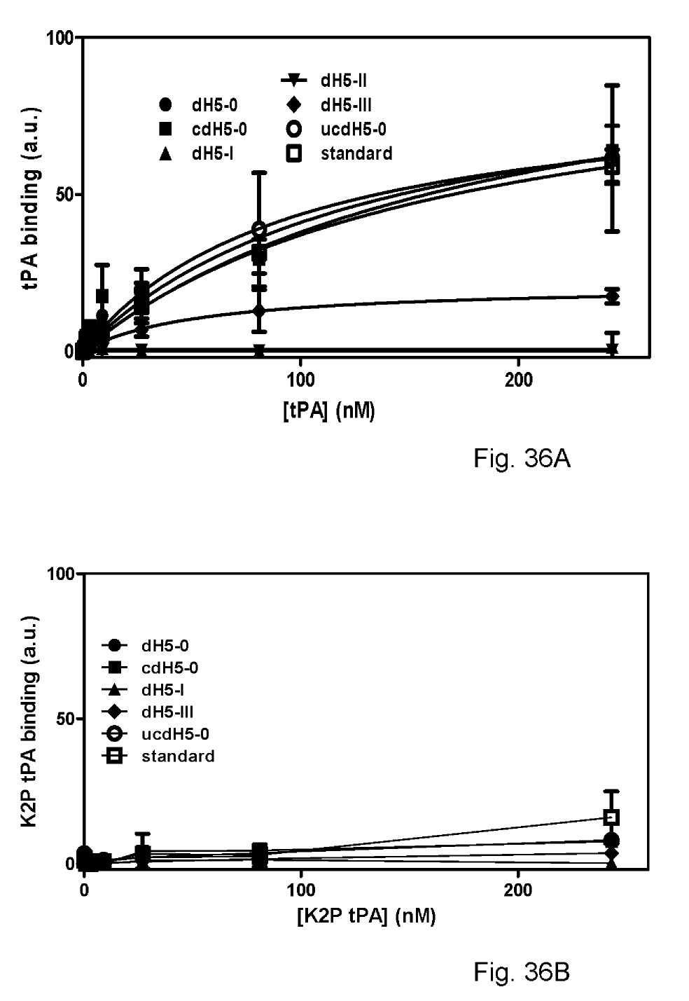

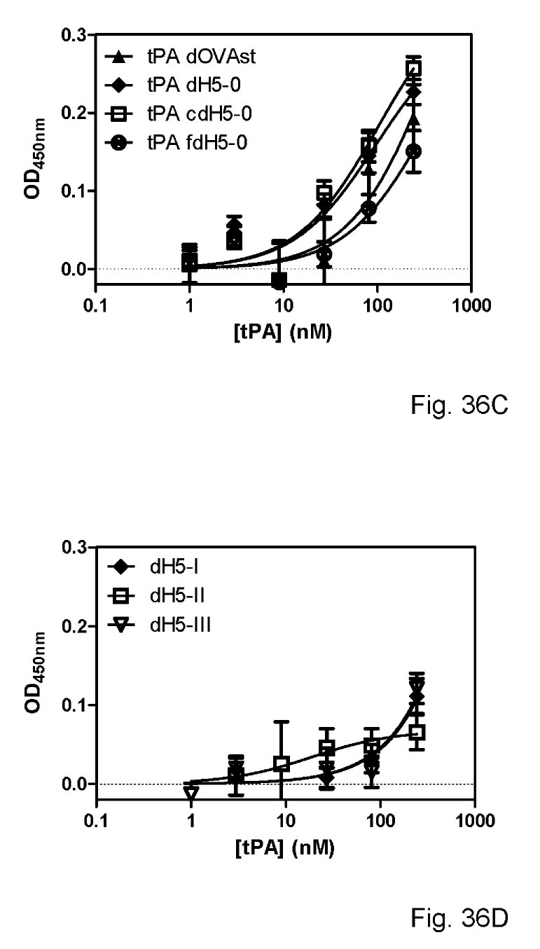

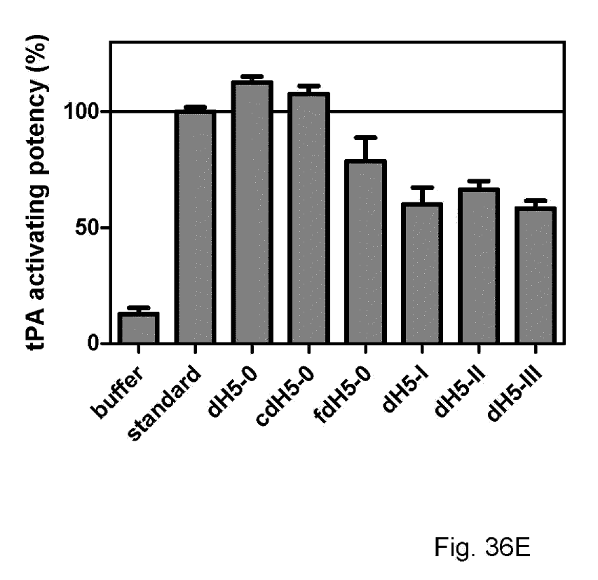

Binding of tPA via its finger domain to various cross-beta comprising H5 forms. In FIGS. 36A, C and D it is seen that tPA binds to a higher order to dH5-0, cdH5-0 and fdH5-0, when compared to dH5-I, dH5-II and dH5-III, indicating that the first three forms expose more tPA binding sites than the latter three forms. Indeed, this is expressed in Bmax values, which is a relative measure for the number of binding sites: Bmax values are 0.32, 0.36 and 0.37 for dH5-0, cdH5-0 and fdH5-0, respectively, whereas the Bmax value could not be determined for dH5-I and dH5-III (too less binding sites), and Bmax is relatively low for dH5-II, i.e., 0.07. The kD values representing the affinity of tPA for the H5 forms, are 96, 102 and 342 nM for dH5-0, cdH5-0 and fdH5-0, respectively. Again, for dH5-I and dH5-III this kD value could not be determined, whereas the relatively few tPA binding sites on dH5-II bind tPA with an affinity of 19 nM. In FIG. 36A it is shown that after ultracentrifugation for 1 hour at 100,000*g of dH5-0 (depicted as “ucdH5-0”) tPA binds with similar affinity and to a similar number of binding sites, showing that the tPA binding fraction in dH5-0 is soluble. With Fn F4-5 a similar tendency with respect to the relative amount of binding sites for finger domains was seen when dH5-0, cdH5-0 and fdH5-0 are compared to dH5-I, dH5-II and dH5-III (see FIG. 35 and Table 13).

tPA/Plg activation by H5 samples comprising cross-beta structure. The six H5 samples were tested for their tPA mediated plasminogen activation potency at a concentration of 50 μg/ml. The results are shown in FIG. 36E. Notably, the activation potency expressed as conversion of plasmin chromogenic substrate, of dH5-0, cdH5-0 and fdH5-0 is similar, and for all three forms higher than the plasmin activity seen with dH5-I, dH5-II and dH5-III. These potencies to activate tPA/plasminogen are in line with the tPA binding data as discussed above and depicted in FIG. 36. It is concluded that the cross-beta structures that are induced in H5 forms dH5-I, dH5-II and dH5-III have less potency to interact with tPA than the cross-beta structures present in dH5-0, cdH5-0 and fdH5-0.

Immunization of mice with six H5 variants, followed by analysis of H5-specific antibodies and T-cell activation analysis. As outlined above, Balb/c mice are immunized twice, at day 0 and day 21, with a dose of 5 μg of the six H5 forms. Group 2, dH5-0; group 3, cdH5-0; group 4, fdH5-0; group 5, dH5-I; group 6, dH5-II; group 7, dH5-III. Controls are group 1, placebo (PBS), and group 8, 5 μg cdH5-0 mixed with 40 times diluted alum (Adjuphos, Brenntag). At day 33 blood is drawn for titer determination (See Table 15). The total anti-H5 antibody titer of IgG and IgM isotypes is determined, in an ELISA using immobilized cdH5-0 and dilution series of the individual mouse sera. At day 41 mice were sacrificed, blood was taken to determine anti-H5 antibody formation and splenocytes were isolated to determine T cell responses.

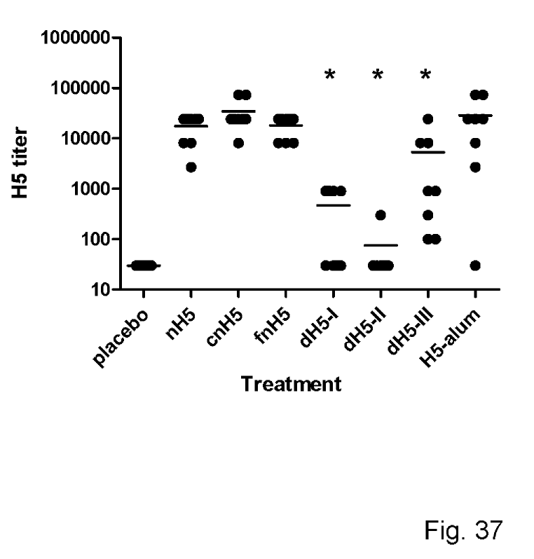

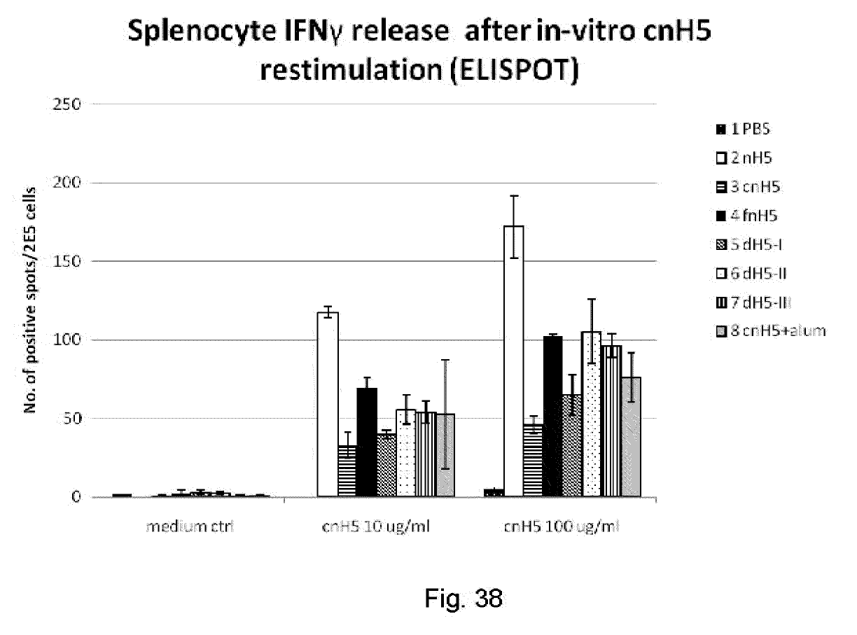

In Table 15 and FIG. 37 the results and observations of the H5 immunizations are depicted. In Table 15, for each individual mouse its anti-H5 antibody titer in sera is given. The data demonstrate that the various structural forms of H5 induce antibody titers to a varying extent.

When mice immunized with dH5-0, cdH5-0 and fdH5-0 are again compared to dH5-I, dH5-II and dH5-III, respectively it is seen that the dH5-0, cdH5-0 and fdH5-0 that are provided with a combination of i) type of cross-beta structure, ii) relative amount of cross-beta structure, iii) relative multimeric molecular distribution, iv) relative fraction of soluble molecules, induce antibodies more efficiently than dH5-I, dH5-II and dH5-III. These latter three forms also induced less protection against H5N1 infection (not shown), and structural and functional parameters differed from those seen with dH5-0, cdH5-0 and fdH5-0.

FIG. 38 shows the results of the analysis of the T cell response, determined by the release of IFNγ using an ELISPOT analysis on the cultured isolated splenocytes ex vivo. The method was identical to that used for the ELISPOT analysis with OVA, except that cdH5-0 was used as stimulus. It is seen that immunogenic composition with H5 induce a T cell response. All H5 immunogenic composition comprising cross-beta structure induce a T cell response with some differences in induction capacity, being dH5-0 the strongest.

This example demonstrates that it is possible to select immunogenic compounds comprising cross-beta structure and T cell epitope that can generate a T cell response, even in the absence of epitopes for specific antibody in the immunogenic composition and in the absence of a humoral response.

TABLE 1
Endotoxin level of various cross-beta OVA forms

| | Endotoxin Level | Endotoxin level of 5 |
|---|---|---|
| Sample | (EU/ml) | μg OVA |
| nOVA | 2.19 | 0.55 |
| dOVA-1 | 5.08 | 1.27 |
| dOVA-2 | 3.03 | 0.758 |
| dOVA-3 | 1.26 | 0.315 |

TABLE 2
Visual inspection of various cross-beta OVA forms

| | Appearance of OVA | Appearance of OVA solution |
|---|---|---|
| Sample | solution | after one freeze/thaw cycle |
| nOVA | Clear | Clear |
| dOVA-1 | Turbid, and big | A bit turbid, big |
| | pellet after 16.000 g | flakes visible |
| dOVA-2 | Clear | Clear |
| dOVA-3 | Clear | A bit turbid |

TABLE 3
Analysis of OVA multimerization by
Transmission Electron Microscopy

| Sample | Appearance of OVA solution |
|---|---|
| Buffer | Empty view |
| nOVA | Empty view |
| dOVA-1 | heterogenous picture, size variation: from small to medium |
| | size aggregates, cloudy appearance, also elongated |
| | structures (fibre like) spotted but not in every |
| dOVA-2 | heterogenous picture, size variation: from small to medium |
| | size aggregates, cloudy appearance |
| dOVA-3 | reasonable uniform picture, size variation: from small to |
| | medium size aggregates, cloudy appearance, very open |
| | structure |

TABLE 4
Enhancement of Thioflavin T fluorescence under
influence of various cross-beta OVA forms.

| Sample | ThT fluorescence (U/mL) |
|---|---|
| dOVA st-100 | 100.00 |
| PBS | 0.00 |
| HBS − NaCl | −3.47 |
| nOVA | 3.31 |
| dOVA-1 | 49.39 |
| dOVA-2 | 62.47 |
| dOVA-3 | 77.74 |

TABLE 5
Enhancement of Sypro Orange fluorescence under
influence of various cross-beta OVA forms.

| | SO fluorescence |
|---|---|
| Sample | (U/mL) |
| dOVA reference standard | 100.00 |
| PBS | 0.00 |
| HBS − NaCl | 0.04 |
| nOVA | 0.90 |
| dOVA-1 | 56.06 |
| dOVA-2 | 48.58 |
| dOVA-3 | 41.44 |

TABLE 6
tPA activation potency of cross-beta OVA samples

| | Activation at 25 | Activation at 10 |
|---|---|---|
| OVA form | μg/mL | μg/mL |
| dOVA-2 80* | 100.00 | 100.00 |
| HBS | 23.35 | |
| PBS | 9.25 | |
| HBS + NaCl | 13.21 | |
| nOVA | 48.14 | 48.78 |
| dOVA-1 | 136.13 | 97.40 |
| dOVA-2 | 106.69 | 107.22 |
| dOVA-3 | 79.04 | 60.91 |

*Reference: Fluorescent signal set at 100%. Other samples are compared with this reference sample.

TABLE 7
Binding of Fn F4-5 to various cross-beta forms
of OVA: binding sites and affinities

| | Normalized number | |
|---|---|---|
| | of binding sites, | Normalized |
| H5 form | Bmax (%) | affinity, kD (%) |
| nOVA | 100.00 | 100.00 |
| dOVA-1 | 291.23 | 136.70 |
| dOVA-2 | 471.10 | 217.06 |
| dOVA-3 | 502.44 | 166.38 |

Remark: a Bmax > 100% indicates that the OVA form exposes more binding sites for Fn F4-5 than nOVA. A kD > 100% indicates that the OVA form exposes binding sites for Fn F4-5 for which Fn F4-5 has lower affinity.

TABLE 8
Binding of functional monoclonals to OVA structural variants

| | Scaled antibody binding (relative number of binding sites Bmax, a.u.) | | | | | | |
|---|---|---|---|---|---|---|---|
| | Antibody | | | | | | |
| OVA | HYB | HYB | HYB | Sigma | MP | MP | Sigma |
| variant | 099-01 | 099-02 | 099-09 | A6075 | 55303 | 55304 | C6534 |
| nOVA | 1.461 | 2.072 | 2.024 | | 0.9760 | 1.494 | 0.9423 |
| dOVA-1 | 1.5 | 2.629 | 1.937 | 1.637 | 1.600 | 0.9278 | 0.9367 |
| dOVA-2 | 0.6330 | 1.844 | 1.780 | | 1.075 | 1.689 | 0.9891 |
| dOVA-3 | 0.3565 | 0.1731 | 0.05829 | | 1.025 | 1.753 | 0.9779 |

TABLE 9
Binding of functional monoclonals to OVA structural variants

| | Scaled antibody binding (relative affinity kD, a.u.) | | | | | | |
|---|---|---|---|---|---|---|---|
| | Antibody | | | | | | |
| OVA | HYB | HYB | HYB | Sigma | MP | MP | Sigma |
| variant | 099-01 | 099-02 | 099-09 | A6075 | 55303 | 55304 | C6534 |
| nOVA | 98.49 | 103.0 | 71.15 | | 184.1 | 107.1 | 84.94 |
| dOVA-1 | 115.3 | 153.9 | 127.4 | 20.83 | 90.18 | 103 | 44.05 |
| dOVA-2 | 129.4 | 117.9 | 85.12 | | 364.1 | 481.6 | 221.1 |
| dOVA-3 | 80.87 | 154.4 | 164.4 | | 332.4 | 524.0 | 286.7 |

TABLE 10
Antigen and immunization scheme

| Group | | |
|---|---|---|
| (n = 10 + | ovalbumin - 4 weekly | |
| 3 mice) | doses 5 μg | Description |
| 1 | Placebo | PBS |
| 2 | nOVA | OVA standard 1 mg/ml in PBS |
| 3 | dOVA-1 | High pH, 37° C., 40 min |
| | | (dOVA-B5) |
| 4 | dOVA-2 | dOVA standard 1 mg/ml |
| 5 | dOVA-3 | 75° C., o/n (dOVA-b-IV) |
| 6 | nOVA + Freund's | OVA standard 1 mg/ml in PBS |
| | Adjuvant | |

TABLE 11
Antibody titers of individual mice

| antigen | mouse # | titer |
|---|---|---|
| Placebo | 386131 | <30 |
| | 386132 | <30 |
| | 386133 | <30 |
| | 386144 | <30 |
| | 386145 | <30 |
| | 386146 | <30 |
| | 386147 | <30 |
| | 386148 | <30 |
| | 386149 | <30 |
| | 386150 | <30 |
| | 386151 | <30 |
| | 386152 | <30 |
| | 386153 | <30 |
| nOVA | 386128 | <30 |
| | 386129 | 810 |
| | 386130 | <30 |
| | 386154 | <30 |
| | 386155 | <30 |
| | 386156 | <30 |
| | 386160 | <30 |
| | 386161 | <30 |
| | 386162 | <30 |
| | 386163 | 2430 |
| | 386164 | <30 |
| | 386165 | <30 |
| | 386166 | <30 |
| dOVA-1 | 386117 | >7290 |
| | 386118 | >7290 |
| | 386119 | >7290 |
| | 386141 | 7290 |
| | 386142 | 810 |
| | 386143 | 810 |
| | 386157 | >7290 |
| | 386158 | 7290 |
| | 386159 | >7290 |
| | 386173 | >7290 |
| | 386174 | >7290 |
| | 386175 | 7290 |
| | 386189 | 7290 |
| dOVA-2 | 386124 | 810 |
| | 386125 | 810 |
| | 386126 | <30 |
| | 386180 | >7290 |
| | 386181 | <30 |
| | 386182 | >7290 |
| | 386183 | 810 |
| | 386184 | 810 |
| | 386187 | >7290 |
| | 386188 | 810 |
| | 386190 | >7290 |
| | 386191 | >7290 |
| | 386192 | >7290 |
| dOVA-3 | 386115 | >7290 |
| | 386121 | >7290 |
| | 386123 | >7290 |
| | 386193 | >7290 |
| | 386194 | >7290 |
| | 386195 | 2430 |
| | 386196 | 270 |
| | 386197 | >7290 |
| | 386198 | >7290 |
| | 386199 | <30 |
| | 386203 | 810 |
| | 386204 | 270 |
| | 386205 | >7290 |
| nOVA + Freunds | 386116 | 2430 |
| | 386120 | 2430 |
| | 386122 | 7290 |
| | 386136 | 2430 |
| | 386137 | >7290 |
| | 386138 | 2430 |
| | 386139 | 810 |
| | 386140 | 810 |
| | 386185 | 2430 |
| | 386186 | 2430 |
| | 386200 | >7290 |
| | 386201 | >7290 |
| | 386202 | 810 |

TABLE 12
Visual inspection by eye and under
a microscope, of various H5 forms

| | | Appearance of H5 solution under a |
|---|---|---|
| cross-beta | Visual appearance | direct light microscope |
| H5 sample | of H5 solution | (supernatant after centrifugation) |
| dH5-0 | Clear | Many bubble/crystal-like appearances; |
| | | colorless |
| cdH5-0 | Clear | relatively small aggregates |
| dH5-I | Turbid | Uniformly distributed amorphous |
| | | shaped aggregates, relatively large |
| dH5-II | Slightly turbid | Uniformly distributed amorphous |
| | | shaped aggregates, smaller than for |
| | | dH5-I |
| dH5-III | Clear | Uniformly distributed amorphous |
| | | shaped aggregates, relatively small |
| ucdH5-0 | Clear, no pellet | Uniformly distributed amorphous |
| | observed | aggregates, relatively small |
| ucdH5-I | Supernatant is | amorphous aggregates |
| | clear, big pellet | |
| ucdH5-II | Supernatant is | Small (tiny) aggregates |
| | clear, small pellet | |
| ucdH5-III | Supernatant is | Clear |
| | clear, small pellet | |

TABLE 13
Binding of Fn F4-5 to various cross-beta
forms of H5: binding sites and affinities

| | Normalized number | |
|---|---|---|
| | of binding sites, | Normalized |
| H5 form | Bmax (%) | affinity, kD (%) |
| dH5-0† | 114 | 103 |
| cdH5-0 | 100 | 100 |
| fdH5-0 | 146 | 69 |
| dH5-I | 1 | 0 |
| dH5-II | 9 | 88 |
| dH5-III | 13 | 6 |

TABLE 14
Summary of structural data for the six H5 structural variants

| | H5 forms group I | H5 forms group II |
|---|---|---|
| | (dH5-0, cdH5-0, | (dH5-I, dH5-II, |
| | fdH5-0) | dH5-III) |
| Visual inspection/ | Relatively less and | More and larger |
| TEM imaging/ | smaller aggregates, | aggregates, <50% |
| SDS-PAGE/ | >50% soluble | soluble |
| solubility of multimers | | |
| ThT fluorescence | +/− | Increased |
| Sypro orange | +/− | increased |
| fluorescence | | |
| tPA and Fn F4-5 | Relatively high | decreased |
| binding, tPA/Plg | | |
| activation | | |
| Functional antibody | Relatively high | Relatively low |
| binding (number of | | |
| binding sites and | | |
| affinity) | | |

TABLE 15
total anti-H5 IgG/IgM titer of mice

| | Titer |
|---|---|
| placebo Group-mouse # | |
| 1-1 | ≦100 |
| 1-2 | ≦100 |
| 1-3 | ≦100 |
| 1-4 | ≦100 |
| 1-5 | ≦100 |
| 1-6 | ≦100 |
| 1-7 | ≦100 |
| 1-8 | ≦100 |
| dH5-0 Group-mouse # | |
| 2-1 | 8100 |
| 2-2 | 2700 |
| 2-3 | 8100 |
| 2-4 | 24300 |
| 2-5 | 24300 |
| 2-6 | 24300 |
| 2-7 | 24300 |
| 2-8 | 24300 |
| cdH5-0 Group-mouse # | |
| 3-1 | 24300 |
| 3-2 | 8100 |
| 3-3 | 24300 |
| 3-4 | 24300 |
| 3-5 | 72900 |
| 3-6 | 24300 |
| 3-7 | 24300 |
| 3-8 | 72900 |
| fdH5-0 Group-mouse # | |
| 4-1 | 24300 |
| 4-2 | 24300 |
| 4-3 | 24300 |
| 4-4 | 8100 |
| 4-5 | 24300 |
| 4-6 | 8100 |
| 4-7 | 24300 |
| 4-8 | 8100 |
| dH5-I Group-mouse # | |
| 5-1 | 900 |
| 5-2 | ≦100 |
| 5-3 | 900 |
| 5-4 | ≦100 |
| 5-5 | 900 |
| 5-6 | ≦100 |
| 5-7 | ≦100 |
| 5-8 | 900 |
| dH5-II Group-mouse # | |
| 6-1 | ≦100 |
| 6-2 | ≦100 |
| 6-3 | ≦100 |
| 6-4 | ≦100 |
| 6-5 | 300 |
| 6-6 | ≦100 |
| 6-7 | |
| 6-8 | |
| dH5-III Group-mouse # | |
| 7-1 | 300 |
| 7-2 | 24300 |
| 7-3 | 900 |
| 7-4 | ≦100 |
| 7-5 | ≦100 |
| 7-6 | 900 |
| 7-7 | 8100 |
| 7-8 | 8100 |
| cdH5-0 + alum Group-mouse # | |
| 8-1 | 72900 |
| 8-2 | 24300 |
| 8-3 | 72900 |
| 8-4 | 24300 |
| 8-5 | 24300 |
| 8-6 | 2700 |
| 8-7 | ≦100 |
| 8-8 | 8100 |

Antigens:
1. Placebo;
2. non-treated H5 (dH5-0);
3. centrifuged H5 (cdH5-0);
4. ultrafiltrated dH5-0 (fdH5-0);
5. dH5-I;
6. dH5-II;
7. dH5-III;
8. cdH5-0 + alum.
A total anti-H5 antibody titer of antibodies of the IgG and IgM type is given as the highest serum dilution that still gave an optical density signal higher than the averaged background signal + 2x the standard deviation of the eight sera of the placebo group 1, at that same dilution.

1. A method for producing an immunogenic composition comprising at least one peptide, polypeptide, protein, glycoprotein, protein-DNA complex, protein-membrane complex and/or lipoprotein, the method comprising:
determining whether a peptide, polypeptide, protein, glycoprotein, protein-DNA complex, protein-membrane complex and/or lipoprotein comprises a T-cell epitope motif;
selecting a peptide, polypeptide, protein, glycoprotein, protein-DNA complex, protein-membrane complex and/or lipoprotein comprising a T-cell epitope motif;
providing a composition comprising the selected peptide, polypeptide, protein, glycoprotein, protein-DNA complex, protein-membrane complex and/or lipoprotein; and
providing the composition with at least one cross-beta structure.

2. A method for producing an immunogenic composition which is capable of activating a T-cell and/or a T-cell response, the immunogenic composition comprising a peptide, polypeptide, protein, glycoprotein, protein-DNA complex, protein-membrane complex and/or lipoprotein comprising a T-cell epitope and/or a T-cell epitope motif, the method comprising providing the composition with at least one cross-beta structure and determining:
whether the degree of multimerization of the peptide, polypeptide, protein, glycoprotein, protein-DNA complex, protein-membrane complex and/or lipoprotein in the composition allows recognition, binding, excision, processing, and/or presentation of a T-cell epitope of the peptide, polypeptide, protein, glycoprotein, protein-DNA complex, protein-membrane complex and/or lipoprotein by an animal's immune system;
whether between 4-75% of the peptide, polypeptide, protein, glycoprotein, protein-DNA complex, protein-membrane complex and/or lipoprotein content of the composition is in a conformation comprising cross-beta structures;
whether the at least one cross-beta structure comprises a property allowing recognition, binding, excision, processing and/or presentation of a T-cell epitope of the peptide, polypeptide, protein, glycoprotein, and/or lipoprotein by an animal's immune system; and/or
whether a compound capable of specifically binding, recognizing, excising, processing, and/or presenting a T-cell epitope of the peptide, polypeptide, protein, glycoprotein, protein-DNA complex, protein-membrane complex and/or lipoprotein is capable of specifically binding, recognizing, excising, processing and/or presenting the T-cell epitope.

3. The method according to claim 1, comprising determining whether an MHC antigen-processing pathway is capable of binding, recognizing, excising, processing, and/or presenting a T-cell epitope of the peptide, polypeptide, protein, glycoprotein, protein-DNA complex, protein-membrane complex and/or lipoprotein.

4. The method according to claim 1, comprising determining whether the cross-beta structure is capable of specifically binding a cross-beta structure binding compound, tPA, BiP, factor XII, fibronectin, hepatocyte growth factor activator, at least one finger domain of tPA, at least one finger domain of factor XII, at least one finger domain of fibronectin, at least one finger domain of hepatocyte growth factor activator, Thioflavin T, Thioflavin S, Congo Red, CD14, a multiligand receptor such as RAGE or CD36 or CD40 or LOX-1 or TLR2 or TLR4, a cross-beta-specific antibody, cross-beta-specific IgG and/or cross-beta-specific IgM, IgIV, an enriched fraction of IgIV capable of specifically binding a cross-beta structure, Low density lipoprotein Related Protein (LRP), LRP Cluster II, LRP Cluster IV, Scavenger Receptor B-I (SR BI), SR A, chrysamine G, a chaperone, a heat shock protein, HSP70, HSP60, HSP90, gp95, calreticulin, a chaperonin, a chaperokine, and/or a stress protein.

5. The method according to claim 1, further comprising:
selecting an immunogenic composition wherein the degree of multimerization of the peptide, polypeptide, protein, glycoprotein, protein-DNA complex, protein-membrane complex and/or lipoprotein allows recognition, binding, excision, processing and/or presentation of a T-cell epitope of the peptide, polypeptide, protein, glycoprotein, protein-DNA complex, protein-membrane complex and/or lipoprotein by an animal's immune system.

6. The method according to claim 1, further comprising:
selecting an immunogenic composition wherein between 4-75% of the peptide, polypeptide, protein, glycoprotein, protein-DNA complex, protein-membrane complex and/or lipoprotein content of the immunogenic composition is in a conformation comprising cross-beta structures.

7. The method according to claim 1, further comprising:
selecting an immunogenic composition which comprises a cross-beta structure which is capable of specifically binding a cross-beta structure binding compound, tPA, BiP, factor XII, fibronectin, hepatocyte growth factor activator, at least one finger domain of tPA, at least one finger domain of factor XII, at least one finger domain of fibronectin, at least one finger domain of hepatocyte growth factor activator, Thioflavin T, Thioflavin S, Congo Red, CD14, a multiligand receptor such as RAGE or CD36 or CD40 or LOX-1 or TLR2 or TLR4, a cross-beta-specific antibody, cross-beta-specific IgG and/or cross-beta-specific IgM, IgIV, an enriched fraction of IgIV capable of specifically binding a cross-beta structure, Low density lipoprotein Related Protein (LRP), LRP Cluster II, LRP Cluster IV, Scavenger Receptor B-I (SR BI), SR A, chrysamine G, a chaperone, a heat shock protein, HSP70, HSP60, HSP90, gp95, calreticulin, a chaperonin, a chaperokine and/or a stress protein.

8. The method according to claim 1, further comprising:
selecting an immunogenic composition wherein a compound capable of binding, recognizing, excising, processing and/or presenting a T-cell epitope, an MHC antigen-processing pathway, is capable of binding, recognizing, excising, processing and/or presenting a T-cell epitope of the peptide, polypeptide, protein, glycoprotein, protein-DNA complex, protein-membrane complex and/or lipoprotein.

9. An in vitro method for selecting, from a plurality of immunogenic compositions comprising at least one cross-beta structure and at least one peptide and/or polypeptide and/or protein and/or glycoprotein and/or protein-DNA complex and/or protein-membrane complex and/or lipoprotein with a T-cell epitope or a T-cell epitope motif, one or more immunogenic compositions having a higher chance of being capable of eliciting a protective prophylactic cellular immune response and/or a therapeutic cellular immune response in vivo, as compared to the other immunogenic compositions of the plurality of immunogenic compositions, the method comprising:
selecting, from the plurality of immunogenic compositions, an immunogenic composition:
wherein the degree of multimerization of the peptide, polypeptide, protein, glycoprotein, protein-DNA complex, protein-membrane complex and/or lipoprotein in the composition allows recognition, binding, excision, processing and/or presentation of a T-cell epitope of the peptide, polypeptide, protein, glycoprotein, protein-DNA complex, protein-membrane complex and/or lipoprotein by an animal's immune system;
wherein between 4-75% of the peptide, polypeptide, protein, glycoprotein, protein-DNA complex, protein-membrane complex and/or lipoprotein content of the composition is in a conformation comprising cross-beta structures;
which comprises a cross-beta structure which is capable of specifically binding a cross-beta structure binding compound, tPA, BiP, factor XII, fibronectin, hepatocyte growth factor activator, at least one finger domain of tPA, at least one finger domain of factor XII, at least one finger domain of fibronectin, at least one finger domain of hepatocyte growth factor activator, Thioflavin T, Thioflavin S, Congo Red, CD14, a multiligand receptor such as RAGE or CD36 or CD40 or LOX-1 or TLR2 or TLR4, a cross-beta-specific antibody, cross-beta-specific IgG and/or cross-beta-specific IgM, IgIV, an enriched fraction of IgIV capable of specifically binding a cross-beta structure, Low density lipoprotein Related Protein (LRP), LRP Cluster II, LRP Cluster IV, Scavenger Receptor B-I (SR BI), SR A, chrysamine G, a chaperone, a heat shock protein, HSP70, HSP60, HSP90, gp95, calreticulin, a chaperonin, a chaperokine and/or a stress protein; and/or
wherein a compound capable of binding, recognizing, excising, processing and/or presenting a T-cell epitope, an MHC antigen-processing pathway, is capable of binding, recognizing, excising, processing and/or presenting a T-cell epitope of the peptide, polypeptide, protein, glycoprotein, protein-DNA complex, protein-membrane complex and/or lipoprotein.

10. The method according to claim 1, wherein the cross-beta structure is induced in at least part of the peptide, polypeptide, protein, glycoprotein, protein-DNA complex, protein-membrane complex and/or lipoprotein.

11. The method according to claim 1, wherein the peptide, polypeptide, protein, glycoprotein, protein-DNA complex, protein-membrane complex and/or lipoprotein is subjected to a cross-beta inducing procedure, a change of pH, salt concentration, temperature, buffer, and/or chaotropic agent concentration.

12. The method according to claim 1, wherein the peptide, polypeptide, protein, glycoprotein, protein-DNA complex, protein-membrane complex and/or lipoprotein is coupled to a cross-beta-comprising compound.

13. The method according to claim 5, further comprising:
producing a vaccine comprising the selected immunogenic composition.

14. The method according to claim 1, comprising determining whether monomers and/or multimers of the peptide, polypeptide, protein, glycoprotein, protein-DNA complex, protein-membrane complex and/or lipoprotein in the immunogenic composition have dimensions in the range of 0.5 nm to 1000 μm, in the range of 0.5 nm to 100 μm, more in the range of 1 nm to 5 μm, and even more in the range of 3-2000 nm.

15. A composition comprising an immunogenic composition produced and/or selected with the method according to claim 1.

16. The composition of claim 15, which is a vaccine.

17. A method of prophylaxis and/or treatment of a disorder caused by a pathogen, tumor, cardiovascular disease, atherosclerosis, amyloidosis, autoimmune disease, graft-versus-host rejection and/or transplant rejection, the method comprising:
utilizing the composition of claim 15 as a pharmaceutical composition for the prophylaxis and/or treatment of said disorder.

18. The method according to claim 17 comprising:
administering to a subject in need thereof a therapeutically effective amount of the immunogenic composition.

19. The method according to claim 18, wherein the subject is a human.

20. The method according to claim 1, wherein the T-cell epitope is a CTL epitope.

21. The method according to claim 1, wherein the T-cell epitope is a T helper cell epitope.

22. The method according to claim 3, wherein the MHC antigen-processing pathway is a MHC I system.

23. The method according to claim 3, wherein the MHC antigen-processing pathway is a MHC II system.

24. The composition of claim 15, further comprising a suitable carrier.

25. (canceled)

26. (canceled)


SEQUENCE LISTING (15 sequences)

SEQ ID NO: 1 - protein, 9 aa, Human immunodeficiency virus
AMQMLKETI

SEQ ID NO: 2 - protein, 9 aa, Influenza virus
IYSTVASSL

SEQ ID NO: 3 - protein, 9 aa, Influenza virus
LYQNPTTYI

SEQ ID NO: 4 - protein, 9 aa, Influenza virus
TYISVGTST

SEQ ID NO: 5 - protein, 9 aa, Influenza virus
KYVKSNRLV

SEQ ID NO: 6 - protein, 9 aa, Influenza virus
DYEELKHLL

SEQ ID NO: 7 - protein, 10 aa, Influenza virus
SYNNTNQEDL

SEQ ID NO: 8 - protein, 10 aa, Influenza virus
TYISVGTSTL

SEQ ID NO: 9 - protein, 10 aa, Influenza virus
KYVKSNRLVL

SEQ ID NO: 10 - protein, 8 aa, Artificial
  MHC class II for RF33
SIINFEKL

SEQ ID NO: 11 - protein, 10 aa, Artificial
  peptide for D011.10
VAAHAEINEA

SEQ ID NO: 12 - protein, 10 aa, Artificial
  peptide for OT-II
AAHAEINEAG

SEQ ID NO: 13 - protein, 17 aa, Artificial
  OVA323-339
ISQAVHAAHAEINEAGR

SEQ ID NO: 14 - protein, 9 aa, Artificial
  OVA257-264
SIINFEKLL

SEQ ID NO: 15 - protein, 571 aa, Influenza A virus
MRPWTWVLLLLLLICAPSYAGSDQICIGYHANNSTEQVDTIMEKNVTVTHAQDILERTHNGKLCDLNGVKPLILRDCSVAGWLLGNPMCDEFINVPEWSYIVEKASPANDLCYPGNFNDYEELKHLLSRINHFEKIQIIPKSSWSNHDASSGVSSACPYLGRSSFFRNVVWLIKKNSAYPTIKRSYNNTNQEDLLVLWGIHHPKDAAEQTKLYQNPTTYISVGTSTLNQRLVPEIATRPKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNTPQRERRRKKRGLFGAIAGFIEGGWQGMVDGWYGYHHSNEQGSGYAADKESTQKAIDGVTNKVNSIINKMNTQFEAVGREFNNLERRIENLNKKMEDGFLDVWTYNAELLVLMENERTLDFHDSNVKNLYDKVRLQLRDNAKELGNGCFEFYHKCDNECMESVKNGTYDYPQYSEEARLNREEISGVKLESMGTYQILAAADYKDHDGDYKDHDIDYKDHDGAAHHHHHH